(19) United States
(12) Patent Application Publication
(10) Pub. No.: US 2026/0341406 A1
(43) Pub. Date: Oct. 29, 2026

(54) ALLELE SPECIFIC EXPRESSION

(71) Applicant: Iovance Biotherapeutics, Inc., San Carlos, CA (US)

(72) Inventors: Fong Chun CHAN, London (GB); Gayle LAM SHANG LEEN, London (GB)

(21) Appl. No.: 19/114,229

(22) PCT Filed: Sep. 6, 2023
(86) PCT No.: PCT/EP2023/074437

§ 371 Date: Mar. 21, 2025

(30) Foreign Application Priority Data
Sep. 23, 2022 (GB) 2213928.1

(51) Int. Cl. G16B 25/10; G16B 40/20

(52) U.S. Cl. CPC G16B 25/10; G16B 40/20

(57) ABSTRACT

Methods of determining whether a tumour-specific mutation is likely to be expressed in a subject are described. The methods comprise obtaining RNA sequence data from one or more samples from the subject comprising tumour genetic material, the RNA sequence data comprising for each of the one or more samples, the number of RNA reads in the sample that show the tumour-specific mutation (b), and the total number of RNA reads at the location of the tumour-specific mutation (d). The methods further comprise determining the likelihoods of the sequence data if the tumour-specific mutation is (i) expressed and (ii) not-expressed. The methods find use in detecting tumour-specific mutations in RNA sequence data, and in identifying neoantigens or providing therapies targeting such neoantigens. Related methods, systems and products are also described.

### FIELD OF THE DISCLOSURE

The present disclosure relates to methods for determining whether a tumour-specific mutated allele is likely to be expressed and for identifying tumour-specific mutated alleles that are expressed in a tumour. The present disclosure also relates to methods and compositions for the treatment of cancer which make use of or target neoantigens.

### BACKGROUND

It is generally accepted that cancer antigens such as those resulting from cancer specific variants (also referred to as “tumour-specific mutations”) represent promising therapeutic targets for immunotherapy provided that they are expressed by cancer cells (see e.g. Heemskerk, Kvitsborg & Schumacher, 2013). However, approaches to determine whether these antigens (also referred to as “neoantigens”) are expressed have been relatively crude. For example, it has been suggested to use whole genome (WGS) or whole exome (WES) sequencing to identify mutations in genomic DNA, then use RNA sequencing to identify genes that are expressed or differentially expressed in tumour material and focus all efforts on those (see e.g. Heemskerk, Kvitsborg & Schumacher, 2013). Similarly, it has been suggested that RNA sequencing data could be used instead of WGS/WES to identify tumour-specific mutations, with the advantage that this is not limited to known genes and can identify e.g. intragenic fusions, novel transcripts, etc. However, this is complicated by factors such as the differing levels of mRNA expression of any transcript in tumour and normal tissues (making comparisons difficult), and it was accepted that the approach was not suitable to identify variants in RNA species present a low level (see e.g. Heemskerk, Kvitsborg & Schumacher, 2013). Thus, there is a need for improved methods to determine the expression of tumour-specific variants.

### SUMMARY

The present inventors have recognised that the simplistic gene centric approach to RNA expression is insufficient in the context of cancer-specific mutations because it combines information from variant and normal transcripts of the gene (both of which may be present in the tumour cells) and provides an aggregate signal from healthy and tumour cells (since tumour samples are typically mixed samples comprising both types of cells, the latter being potentially genetically heterogeneous), altogether providing very uncertain information about whether a variant is in fact expressed. The present inventors have discovered that an approach that simply looks at gene expression level in a tumour sample as a filter for candidate neoantigens unnecessarily excludes many variants that may in fact be expressed. Thus, the present inventors have recognised that there is a need for a more sensitive approach that can take into account all of the factors mentioned above to more confidently identify the presence of variants in RNA expression data. In particular, the inventors have devised an approach to determine the probability that a tumour-specific mutation is expressed in a tumour using RNA sequence data from one or more samples comprising tumour cells or genetic material derived therefrom, as well as an approach to determine whether the RNA sequence data that is available provides sufficient information to determine whether a tumour-specific mutation is expressed in a sample. This method finds particular use in identifying neoantigens, for example for the purpose of cancer therapy or prognosis.

In particular, when assessing whether a T cell reaction can be observed in vitro for a plurality of tumour-specific mutations identified e.g. from genomic sequence data, the present inventors have identified that many mutations that do trigger a reaction are not identified as expressed in RNA-seq data. In other words, the inventors have identified the presence of a subset of mutations for which no expression is detected, but which were found to be immunogenic. There are multiple possible explanations for this. For example, it is possible that these mutations are immunogenic but not expressed due to immuno-editing. In other words, it is possible that there is truly no expression of the mutation. Another possibility is that the variant is expressed but there is low power to detect expression at this locus in the RNA-seq data. In order to be able to distinguish between these two situations, the present inventors devised a method to calculate the power to detect a mutation in expression data. This method is particularly useful to identify whether known mutations that may be expressed at low levels (where the mutation and/or the locus is expressed at low level and/or the tumour purity of the sample is low) are truly not expressed or likely false negatives (mutations that may be expressed but are not detected in the data at hand). The method can also be used to identify mutations directly from RNA expression data. This may be particularly useful to identify splicing variants such as e.g. retained introns and skipped exons, or any variant that cannot be straightforwardly identified using genomic data for example due to mappability problems such as fusions. The inventors evaluated the method using a cell line titration series in which high confidence variants (in particular, single nucleotide variants) could be identified in the pure sample and checked for expression (>=1 variant read in pure sample), and their expression assessed in dilution series with 50%, 20% or 10% variant cells. This showed that false negative mutations (i.e. mutations that are expressed by the cell line but for which no expression is detected in a dilution sample) have low predicted power to detect allele specific expression determined according to the new method. The method uses a rigorous statistical framework to classify individual mutations as expressed, and provides a probability reflecting the confidence in the assignment. The method is fast, flexible, robust and replicable, relies on interpretable assumptions, and can flexibly incorporate genotype and purity data in providing its predictions.

Thus, according to a first aspect, there is provided a method of determining whether a tumour-specific mutation is likely to be expressed in a subject, the method comprising: providing, or obtaining, RNA sequence data from one or more samples from the subject comprising tumour genetic material, the RNA sequence data comprising for each of the one or more samples: the number of RNA reads in the sample that show the tumour-specific mutation (b), and the total number of RNA reads at the location of the tumour-specific mutation (d); and determining the likelihoods of the sequence data if the tumour-specific mutation is (i) expressed (P(b,d|α,t,E=1)) and (ii) not-expressed (P(b,d|α,t,E=0)). The likelihoods of the sequence data may also be referred to as the probability of observing the sequence data if the mutation is expressed, and the probability of observing the sequence data if the mutation is not expressed.

The method of the present aspect may have one or more of the following features.

The likelihoods may be the probabilities of the sequence data in view of (conditional on) a tumour fraction for each of the one or more samples, and the fraction of the total number of reads at the location of the tumour-specific mutation that originate from a population of cells in the one or more samples that does not comprise the tumour-specific mutation (e.g. normal cells), the samples further comprising a population of cells that comprises the tumour-specific mutation (e.g. tumour cells) representing a fraction of the sample equal to the tumour fraction. The likelihoods may depend on the probability of sampling a sequence read comprising the tumour-specific mutation from a sample if the tumour-specific mutation is expressed or not expressed, respectively, depending on the sequencing error rate, the genotypes of the tumour and normal cell populations, the tumour fraction of the sample, and the fraction of total read counts for the gene comprising the tumour-specific mutation which is due to the normal cell population. The likelihoods of the sequence data may be the probabilities of observing the sequence data in view of (conditional on) a tumour fraction for each of the one or more samples (t), and the fraction (a) of the total number of reads at the location of the tumour-specific mutation that originate from a population of cells in the one or more samples that does not comprise the tumour-specific mutation. The samples may therefore comprise a population of cells that does not comprise the tumour-specific mutation and a tumour population of cells that comprises the tumour-specific mutation, the latter representing a fraction of the sample equal to the tumour fraction. The likelihoods may be determined using equations (5) or (5′).

The method may further comprise comparing the likelihoods obtained thereby determining whether the tumour-specific mutation is likely to be expressed in the subject. Comparing the likelihoods may comprise determining the posterior probability that the tumour-specific mutation is expressed (P(E=1|b,d,α,t)) depending on: a prior probability of the mutation being expressed (μ_{ρ}), and the likelihoods of the sequence data if the tumour-specific mutation is (i) expressed (P(b,d|α,t,E=1)) and (ii) not-expressed (P(b,d|α,t,E=0)). The posterior probability may be determined using any of equations (13) (in particular any of the different formulations of P(E=1|b,d,α,t) provided by equation (13)), (14) or (14′) (in particular any of the different formulations of P(E=1|b,d,α,t) provided by equations (14) or (14′)). Comparing the likelihoods may comprise determining the power to detect whether the tumour-specific mutation is expressed at a predetermined false positive rate, wherein the power to detect whether the tumour-specific mutation is expressed is the area under the curve of the likelihood of the number of reads that show the tumour specific mutation if the tumour-specific mutation is expressed (P(b,d|α,t,E=1), P(b|E=1), P(b|M₁)) as a function of the number of RNA reads in the sample that show the tumour-specific mutation (b), above a threshold number of reads (b_{c}), wherein the threshold number of reads (b_{c}) is the number of reads such that: the area under the curve of the likelihood of the number of reads that show the tumour specific mutation if the tumour-specific mutation is not expressed (P(b,d|α,t,E=0), P(b|E=0), P(b|M₀)) as a function of the number of RNA reads in the sample that show the tumour-specific mutation (b) above the threshold number of reads is equal to the predetermined false positive rate. The power to detect a mutation as being expressed may be determined using equation (8), where P(b|M₁) is the likelihood of the sequence data if the mutation is expressed, which may be defined in equation (5) or (5′). The choice of a false positive rate may depend on the situation and the user's preferences. For example, a false positive rate of 0.05 or 0.01 may be chosen. The method may further comprise determining whether the number of RNA reads in the sample that show the tumour-specific mutation (b) is below or above the threshold number of reads (b_{c}).

The inventors have devised a Bayesian framework for determining the probability that a tumour specific mutation is expressed, and a statistical hypothesis testing framework to decide whether a tumour specific mutation is expressed and quantify the power of this test. Both frameworks are based on quantifying the likelihood of an observed number of RNA reads comprising the mutation (in the context of a total number of RNA reads at the location of the tumour specific mutation) if the tumour-specific mutation is expressed, and the likelihood of an observed number of RNA reads comprising the mutation (in the context of a total number of RNA reads at the location of the tumour specific mutation) if the tumour-specific mutation is not expressed. These are then compared to obtain a posterior probability (which depends on the likelihood ratio), or compared to quantify the true positive rate associated with a threshold number of reads comprising the mutation that is required to call the tumour specific mutation as expressed. Thus, both frameworks enable to rigorously determine whether a tumour specific mutation is expressed in view of the data about expression at the locus of the mutation, using quantities and parameters that are directly interpretable and linked to biological phenomena. The Bayesian framework additionally can incorporate an informative prior, such as e.g. knowledge of whether the gene comprising the tumour-specific mutation is expected to be expressed, whether mutations in this disease type are typically expressed, etc.

A tumour-specific mutation may be considered to be likely to be expressed if the posterior probability that the tumour-specific mutation is expressed is above a predetermined threshold. A tumour-specific mutation may be considered to be unlikely to be expressed if the posterior probability that the tumour-specific mutation is expressed is below a predetermined threshold. The predetermined threshold may be selected as the threshold that maximise the true positive rate while keeping the false positive rate below a predetermined value (such as e.g. 0.05) in a set of tumour-specific mutations with known expression status. The set of tumour-specific mutations with known expression status may be obtained using one or more pure tumour samples (e.g. samples with tumour purity=1). Such samples may be obtained by purification or one or more tumour cell lines. The predetermined threshold may be approximately 0.5, between 0.4 and 0.7, or between 0.5 and 0.6. A tumour-specific mutation may be considered to be likely to be expressed if the power to detect whether the tumour-specific mutation is expressed is above a predetermined threshold and the number of RNA reads in the sample that show the tumour-specific mutation (b) is above a threshold number of reads (b_{c}). A tumour-specific mutation may be considered to be unlikely to be expressed if the power to detect whether the tumour-specific mutation is expressed is above a predetermined threshold and the number of RNA reads in the sample that show the tumour-specific mutation (b) is below a threshold number of reads (b_{c}). A tumour-specific mutation may be considered to be likely to be expressed if the power to detect whether the tumour-specific mutation is expressed is below a predetermined threshold and the number of RNA reads in the sample that show the tumour-specific mutation (b) is below a threshold number of reads (b_{c}). In such cases, the RNA sequence data may be insufficient to determine confidently whether the tumour-specific mutation is truly expressed or not, and the tumour-specific mutation may therefore be considered likely to be expressed by default. This is by contrast to previous methods that would simply ignore tumour-specific mutations that are detected at low level in RNA sequence data, regardless of whether this low level is truly indicative of a lack of expression or instead results from limitations of the data itself.

Obtaining RNA sequence data comprising the number of RNA reads in the sample that show the tumour-specific mutation (b), the number of RNA reads in the sample that show the corresponding germline allele, and the total number of RNA reads at the location of the tumour-specific mutation (d) may instead comprise obtaining RNA sequence data comprising at least two of: the number of RNA reads in the sample that show the tumour-specific mutation (b), the number of RNA reads in the sample that show the corresponding germline allele(s) (or do not show the tumour-specific mutation), and the total number of RNA reads at the location of the tumour-specific mutation (d). The RNA sequence data may comprise RNA sequence data from a plurality of samples from the subject. The plurality of samples may be tumour samples. The posterior probability that the tumour specific mutation is expressed may be a posterior probability that the tumour specific mutation is expressed ubiquitously in a tumour of the subject. Such a posterior probability may be determined using equation (14′) (and in particular any of the formulations of equation (14′)).

Each sample may be assumed to comprise: a first population of cells that each have a genotype (G_{V}) comprising at least one copy of the tumour-specific mutation, and a second population of cells that each have a genotype that does not comprise the tumour-specific mutation (G_{N}), the first population of cells representing a proportion equal to the tumour fraction (t) for the respective sample. The likelihoods may be obtained as sums over a plurality of joint genotypes comprising a genotype for the first population of cells and a genotype for the second populations of cells. The sum may be a weighted sum, wherein the likelihoods assuming each of the respective joint genotypes are weighted by a probability associated with the joint genotype. The probabilities associated with each of the respective joint genotypes may sum to 1. The second population of cells may be assumed to have a homozygous diploid reference allele genotype (AA). The joint genotypes may be determined using estimates of the major and minor copy numbers for the locus, derived from DNA sequence data from the sample or a related sample. The joint genotypes may be determined from estimates of the major and minor copy numbers for the locus by assuming that the possible genotypes of the first population of cells include any genotype with a copy number equal to the sum of the major and minor copy number and a number of copies of the variant allele between the minor copy number and the major copy number. The possible genotypes of the first population of cells may include genotypes assuming that the mutation to the variant allele occurred prior to or before any copy number event (decrease or increase of copy number at the locus compared to a diploid genotype). Each of the possible genotypes considered may be associated with an equal probability. The tumour-specific mutation may be assumed to be ubiquitous in the one or more samples. The tumour-specific mutation may be a clonal mutation or a mutation assumed to be clonal in the tumour of the subject. The method may comprise determining whether the tumour-specific mutation is likely to be clonal in the subject. Thus, also described herein is a method of determining whether a clonal tumour-specific mutation is likely to be expressed in a subject.

The likelihoods of the sequence data if the tumour-specific mutation is (i) expressed and (ii) not expressed may depend on the fraction (a) of the total expression of the gene comprising the tumour-specific mutation (number of reads or transcripts per million, TPM) that originate from a population of cells in the one or more samples that does not comprise the tumour-specific mutation. References to the total expression of the gene comprising the tumour-specific mutation may refer to the total expression of one or more transcripts derived from the gene. The parameter α may be set to a predetermined default value or wherein α is estimated from data comprising RNA sequence data for a plurality of samples having different tumour fractions. For example, a may be set to a predetermined default value of α=0.5. This is equivalent to assuming that the tumour and normal cells contribute equal amounts to the total expression at the locus. Alternatively, a may be set to a value that depends on the expression level for the gene in one or more tumours and normal samples. The expression level for the gene in one or more tumours and normal samples may be obtained from a database. For example, expected expression of the gene in samples from the same tumour type and normal samples may be used, or differential expression between one or more samples from the same tumour type and normal samples. When the genotype of a population of cells in the one or more samples that have a genotype comprising at least one copy of the tumour-specific mutation does not have any non-mutant alleles at the locus of the tumour-specific mutation, a may be set to 1 when determining the likelihood of the sequence data if the tumour-specific mutation is not-expressed. The parameter α may be estimated from data comprising RNA sequence data for a plurality of samples having different tumour fractions, wherein α is derived from the slope (g) of a regression model (e.g. a linear regression model) fitted to the total expression of the gene comprising the tumour-specific mutation in a plurality of samples as a function of their tumour purities. The value of the parameter α may be provided by

$$α = \frac{1}{2} ( 1 - \frac{g}{T P {M}^{T} + T P {M}^{N}} )$$

where TPM^{T} and TPM^{N} are the total expression values for the gene comprising the tumour-specific mutation for a tumour sample and a normal sample, respectively. The values of TPM^{T} and TPM^{N} may be obtained using a regression model fitted to values of the total expression for the gene (TPM) in a plurality of tumour samples with at least two different tumour fractions (t), as a function of tumour fraction. The parameter α is preferably set or estimated on a gene or transcript basis, i.e. using data or prior knowledge about the particular gene or transcript comprising the tumour-specific mutation. The parameter a may be estimated using expression data from the same subject, or from subjects having the same type of tumour as the subject.

The parameter may be estimated individually for the gene i comprising the tumour-specific mutation. Thus, the equation above may also be written as

$${α}_{i} = \frac{1}{2} ( 1 - \frac{g}{T P {M}_{i}^{T} + T P {M}_{i}^{N}} ) .$$

The regression model may be a linear regression model. The values of TPM^{T} and TPM^{N} may be obtained as the value of TPM estimated from the regression model at t=1 and t=0, respectively.

The probability of the tumour-specific mutation being expressed may be the mean μ_{ρ} of a Beta distribution (p(ρ) for parameter ρ of a Bernoulli distribution for the variable capturing whether the tumour-specific mutation is expressed (E). The parameter μ_{ρ} may be set to a predetermined value. The parameter μ_{ρ} may be set to a first predetermined value when the total number of RNA reads at the location of the tumour-specific mutation is above or at or above a predetermined threshold. The predetermine threshold may be 0 (d>0). When the total number of RNA reads at the location of the tumour-specific mutation is below a predetermined threshold, μ_{ρ} may be set to a first predetermined value or a second predetermined value depending on whether the gene comprising the tumour-specific mutation is expected to be expressed. The gene comprising the tumour-specific mutation may be considered as expected to be expressed when the total expression of the gene in the sample is above a predetermined threshold, and considered as expected not to be expressed otherwise. The first predetermined value of μ_{ρ} may be 0.5. The second predetermined value of μ_{ρ} may be below 0.5, such as e.g. 0.2, 0.1, 0.05, or 0.01. The predetermined threshold on total expression of the gene comprising the tumour-specific mutation may be 1 TPM (transcript per million). The predetermined threshold on the total number of RNA reads at the location of the tumour-specific mutation may be 0. The predetermined threshold on total expression of the gene comprising the tumour specific mutation may be set depending on the expected level of expression of the gene in the one or more tumour samples, such as e.g. based on the expression level of the gene in one or more further tumour samples, such as e.g. tumour samples from the same type of tumour as that of the subject. The expression level of the gene in one or more further tumour samples may be obtained e.g. from a database. A value for the prior probability of the mutation being expressed may depend on the subject, the tumour, the mutation, or a combination of these. For example, a value may be determined using data previously acquired on a relevant cohort of patients, such as e.g. patients that suffer from the same type or subtype of cancers. Alternatively, a value may be set arbitrarily based on prior knowledge about the cancer type or mutation. For example, specific mutations that have been found across a plurality of cancer samples and have been identified as often being expressed in these samples may be assigned a higher than 0.5 probability. Thus, the present inventors have adapted a simple Bayesian framework with a single prior to instead include, in cases where there is insufficient evidence from the data (i.e. the total number of reads at the locus of the tumour-specific mutation is below a predetermined threshold), a prior that has multiple possible values including at least a first value if there is evidence that the gene is expressed, and a second value if there is not enough evidence that the gene is expressed. For example, if TPM>1=then the gene may be assumed to be expressed, and the prior may be set to 0.5. This reflects the assumption that the lack of detection of reads at the locus (whether for the variant or reference allele) is likely the result of a technical problem rather than being indicative of whether the tumour-specific mutation is expressed. If TPM=0 at the locus then the gene may be assumed not to be expressed, and the prior may be set to a value <0.5. This reflects the assumption that if the gene is not expressed then the tumour-specific mutation is also unlikely to be expressed.

The likelihoods may be conditional on the tumour purity of the one or more samples. The tumour purity of the one or more samples may be estimated using genomic sequence data from the one or more samples. For example, the tumour purity may be estimated using methods known in the art such as e.g. ASCAT or Sequenza. The tumour purity for a sample may be the purity with highest confidence obtained from a probabilistic method for estimating tumour purity from DNA sequence data, such as e.g. a maximum a posteriori estimate. The genotypes of the first and second populations of cells (also referred to as “joint genotype”) may be set to predetermined genotypes, such as e.g. the first population of cells may be considered to be heterozygous diploid comprising a copy of the tumour-specific mutation and a copy of a non-mutated allele, and the second population of cells may be considered to be diploid with two non-mutated alleles. Alternatively, the genotypes of the first and second populations of cells may be estimated using methods known in the art such as e.g. ASCAT or Sequenza. The genotypes for a sample may be the genotypes with highest confidence obtained from a probabilistic method for estimating genotypes in a mixed population from DNA sequence data, such as e.g. a maximum a posteriori estimate joint genotype. The probability of observing the sequence data may depend on the genotypes of the first and second populations of cells, the total number of reads at the locus of the tumour specific mutation, the tumour fraction (t), the fraction (a) of the total expression of the gene comprising the tumour-specific mutation that originate from the second population of cells, and the ratio of expression of the allele(s) not comprising the tumour-specific mutation relative to the total expression at the locus of the tumour-specific mutation (θ). The ratio of expression of the alleles not comprising the tumour-specific mutation relative to the total expression at the locus of the tumour-specific mutation (θ) may be assumed to be a random variable with a first distribution if the tumour-specific mutation is expressed (i.e. for the purpose of estimating the likelihood of the data if the tumour-specific mutation is expressed) and a second distribution if the tumour-specific mutation is not expressed (i.e. for the purpose of estimating the likelihood of the data if the tumour-specific mutation is not expressed). The second distribution may be a beta distribution with parameters α₀, β₀, and the first distribution may be a beta distribution with parameters α₁, β₁. Any of the following parameters may be used alone or in combinations: α₀>1 (e.g. α₀=9999), β₀=1, α₁=1, and β₁=1.

The likelihoods may be marginal likelihoods obtained from the probability of observing the sequence data by integrating out the ratio of expression of the alleles not comprising the tumour-specific mutation relative to the total expression at the locus of the tumour-specific mutation (θ). Thus, the step of determining the likelihoods may comprise a numerical integration, e.g. by a processor, of the probability of observing the sequence data over all possible values (i.e. between 0 and 1) of the ratio of expression of the alleles not comprising the tumour-specific mutation relative to the total expression at the locus of the tumour-specific mutation (θ). In other words, the likelihoods may be integrals over all possible values of 0 of the probability of observing the sequence data multiplied by the distribution of the ratio of expression of the alleles not comprising the tumour-specific mutation relative to the total expression at the locus of the tumour-specific mutation (θ). The likelihoods may be obtained as sums of integrals (e.g. sums of marginal likelihoods) over a plurality of possible genotypes of the first and second population of cells. The sums may be weighted by the respective probabilities of the plurality of possible genotypes.

The probability of observing the sequence data conditional on the genotypes of the first and second populations of cells (G_{V},G_{N}), the total number of reads at the locus of the tumour specific mutation, the tumour fraction (t), the fraction (α) of the total expression of the gene comprising the tumour-specific mutation that originate from the second population of cells, and the ratio of expression of the alleles not comprising the tumour-specific mutation relative to the total expression at the locus of the tumour-specific mutation (θ) may be assumed to follow a binomial distribution with a parameter ξ(G,θ,α,t) representing the probability of sampling a read with the tumour specific mutation from a tumour sample, wherein ξ(G,θ,α,t) is given by any of equations (2), (2′), (2″), optionally wherein

$$ξ ( G , θ , α , t ) = \frac{α ( 1 - t ) c ( {G}_{N} )}{α ( 1 - t ) c ( {G}_{N} ) + ( 1 - α ) t c ( {G}_{V} )} μ ( {G}_{N} , θ , ε ) + \frac{( 1 - α ) t c ( {G}_{V} )}{α ( 1 - t ) c ( {G}_{N} ) + ( 1 - α ) t c ( {G}_{V} )} μ ( {G}_{V} , θ , ε ) \qquad ( {2}^{″} )$$

where c(G_{V}) and c(G_{N}) are respectively the total number of copies of the locus comprising the tumour-specific mutation in the first and second populations of cells,ε is a sequencing error rate, μ(G_{N},θ,ε) is the probability of sampling a read with the tumour-specific mutation from the second population of cells and μ(G_{V},θ,ε) is the probability of sampling a read with the tumour-specific mutation from the first population of cells. The terms μ(G_{N},θ,ε) and μ(G_{V},θ,ε) may be provided by equation (1), respectively with G=G_{N} and G=G_{V} being the genotype of the second and first population of cells, and b(G) being the number of copies of the allele comprising the tumour-specific mutation in the cells of the respective population of cells. The posterior probability may depend on the ratio (r) of the likelihoods of the sequence data if the tumour-specific mutation is (i) expressed (P(b,d|α,t,E=1)) and (ii) not-expressed (P(b,d|α,t,E=0)). The posterior probability may be given by equation (13), (14) or (14′) (i.e. the posterior probability may be equal to

$$\frac{{μ}_{ρ}}{( \frac{1 - {μ}_{ρ}}{r} + {μ}_{ρ} )} )$$

where μ_{ρ} is the prior probability of the mutation being expressed. The prior probability of the mutation being expressed may be the mean of a Beta probability distribution for parameter ρ (p(ρ)), where the variable representing whether the variant is expressed is assumed to follow a Bernoulli distribution with parameter ρ.

The method may be computer implemented. Thus, the step of obtaining the RNA sequence data may be performed by a processor, and the step of determining the likelihoods may be performed by said processor. The step of obtaining the RNA sequence data may comprise receiving RNA sequence data comprising sequence reads from one or more samples from the subject, and determining from said sequence reads at least two of: the number of RNA reads in the sample that show the tumour-specific mutation (b), the number of reads in the sample that show the corresponding germline allele(s) (or do not show the tumour-specific mutation), and the total number of reads at the location of the tumour-specific mutation (d).

The step of determining the posterior probability that the tumour-specific mutation is expressed and/or the power to detect a tumour-specific mutation that is expressed may be computer implemented. The step of determining the posterior probability that the tumour-specific mutation is expressed and/or the power to detect a tumour-specific mutation that is expressed may comprise a step of numerical integration to obtain the likelihoods. In particular, the step may comprise determining the posterior probability that the mutation is expressed in view of a prior probability of the mutation being expressed, and the probabilities of observing the sequence data if the tumour-specific mutation is (i) expressed and (ii) not expressed, by solving a plurality of one dimensional integrals (such as e.g. a pair of integrals for each sample, respectively representing the assumption that the mutation is expressed and not expressed) integrating the probability of the observed sequence data over all possible values of the proportion of RNA reads from the tumour cells in the sample that comprise the tumour-specific mutation (θ). These numerical integrals may be solved independently (such as e.g. in parallel) for each sample, each mutation, and each model (i.e. assuming that the tumour specific mutation is (i) expressed, and (ii) not expressed). Similarly, the step of determining the power to detect a tumour-specific mutation that is expressed may comprise solving a plurality of one dimensional integrals (such as e.g. a pair of integrals for each sample, respectively representing the assumption that the mutation is expressed and not expressed) integrating the probability of the observed sequence data over all possible values of the proportion of RNA reads from the tumour cells in the sample that comprise the tumour-specific mutation (θ). The step of providing may comprise one or more steps, all or some of which are computer implemented.

The method may further comprise obtaining or providing, for each sample, at least one estimate of the tumour fraction, and optionally at least one corresponding set of one or more candidate joint genotypes comprising a genotype for a population of cells that does not comprise the tumour-specific mutation (e.g. a normal cell population) and a genotype for a population of cells that does comprise the tumour-specific mutation (e.g. a tumour population). A tumour fraction estimate may be obtained using a method for determining allele-specific copy number profiles in samples comprising a mixture of tumour and normal cells. Methods for doing this using genomic sequencing or array data are known in the art, for example by expressing the allele specific data as a function of parameters including allele-specific copy numbers, tumour aneuploidy and tumour cell fraction, and identifying the value of these parameters that best fit all of the data. Examples of such methods include e.g. ASCAT (Van Loo et al., 2010), amongst others. Alternatively, a tumour fraction estimate may be determined experimentally. Thus, the method may further comprise obtaining a tumour fraction estimate for each of the one or more samples. In particular, the method may comprise obtaining, by a processor, for each sample, at least one estimate of the tumour fraction comprises the processor determining an estimate of the tumour fraction and allele specific copy numbers using genomic sequence data from the one or more samples, and determining, by said processor, a set of one or more candidate joint genotypes associated with said allele specific copy numbers. A set of one or more candidate genotypes may be obtained using allele-specific copy numbers or variables derived therefrom (or conversely, from which such allele-specific copy numbers can be derived, such as B allele fraction and log R) for the tumour cells in a mixed sample. Allele-specific copy numbers for the tumour cells in a mixed sample may be obtained using a method for determining allele-specific copy number profiles in samples comprising a mixture of tumour and normal cells, such as e.g. ASCAT (Van Loo et al., 2010), Sequenza (Favero et al., 2015), or ascatNgs (Raine et al., 2016), amongst others. Thus, the method may further comprise obtaining, for each of the one or more samples, estimates for at least two of: the copy number of the major allele in the tumour cells in the sample, the copy number of the minor allele in the tumour cells in the sample, and the total copy number at the location of the tumour-specific mutation in the tumour cells in the sample. The estimates of copy number in the tumour cells in the sample may represent a summarised (e.g. average) estimate over the entire population of tumour cells in the sample.

The method may further comprise repeating the method for a plurality of tumour-specific mutations identified in the subject. The method may further comprise ranking or otherwise prioritising the plurality of tumour-specific mutations at least in part based on their determined probability of being expressed in the subject. The method may further comprise identifying one or more tumour-specific mutations in the subject. Identifying one or more tumour-specific mutations in the subject may be performed using genomic or transcriptomic sequence data from one or more samples from the subject comprising tumour genetic material and sequence data from one or more germline samples from the subject, such as by comparing said sequence data. Identifying one or more tumour-specific mutations in the subject may comprise aligning sequence data from at least one sample comprising tumour genetic material to a reference sequence and identifying positions where the sequence of the sample differs from the reference sequence. The method may further comprise aligning sequence data from at least one germline sample to the reference sequence and identifying positions where the sequence of the sample comprising tumour genetic material differs from the germline sample. The reference sequence may be a reference genome or a reference transcriptome.

The step of providing sequence data from one or more samples from the subject may comprise or consist of receiving sequence data from a user (for example through a user interface), from one or more computing device(s), or from one or more data stores or databases. The step of providing sequence data (whether genomic sequence data or RNA sequence data or transcriptomic sequence data) may further comprise sequencing (or otherwise determining the sequence composition of genetic material present in a sample) one or more samples from the subject comprising tumour genetic material. The method may further comprise sequencing (or otherwise determining the sequence composition of genomic material present in a sample) one or more germline samples from the subject. The method may further comprise obtaining, from the subject, one or more samples comprising tumour genetic material and optionally one or more germline samples. Genetic material as used herein comprises RNA molecules (e.g. mRNA transcripts), and optionally DNA molecules (e.g. genomic DNA). The method may further comprise providing to a user, for example through a user interface, the determined probability of the tumour-specific mutation being expressed and/or a value derived therefrom or associated therewith, such as the likelihoods of the sequence data assuming that the tumour specific mutation is expressed or not expressed, the relative likelihood of the sequence data assuming that the tumour specific mutation is expressed or not expressed, and/or the power to detect the tumour specific mutation as expressed given the distributions of the likelihood of the sequence data assuming that the tumour specific mutation is expressed and not expressed, at one or more false positive rates. For example, the method may comprise providing a “expressed status” flag or value based on the determined probability of the tumour-specific mutation being expressed, and/or the values of the likelihoods and power to detect a tumour-specific mutation that is expressed. As another example, the method may comprise providing information identifying the mutation (such as e.g. the sequence of the mutation and its genomic location). According to a further aspect, there is provided a method of identifying one or more neoantigens in a subject, the method comprising: identifying a plurality of tumour-specific mutations in the subject (such as for example, using genomic and/or transcriptomic data from one or more tumour samples from said subject); determining whether one or more of the tumour-specific mutations is likely to be expressed in a tumour of the subject using the method of any embodiment of the preceding aspect; and determining whether one or more of the tumour-specific mutations is likely to give rise to a neoantigen, wherein α neoantigen is a tumour-specific mutation that satisfies one or more predetermined criteria on whether the tumour-specific mutation is likely to be expressed in the tumour and optionally one or more further criteria on whether the tumour-specific mutation is likely to give rise to a neoantigen. Also described according to the present aspect is a method of identifying one or more neoantigens in a subject, the method comprising: identifying, by a processor using sequence data from one or more samples from said subject, a plurality of tumour-specific mutations in the subject; determining, by a processor whether one or more of the tumour-specific mutations is likely to be expressed in the subject using the method of any preceding claim; and selecting, by said processor, one or more of the tumour-specific mutations as candidate neoantigens, wherein a candidate neoantigen is a tumour-specific mutation that satisfies at least one or more predetermined criteria on whether the tumour-specific mutation is likely to be expressed and optionally one or more criteria on whether the tumour-specific mutation is likely to give rise to a neoantigen.

The method of the present aspect may have any one or more of the following features.

A neoantigen may be a tumour-specific mutation that satisfies at least a criterion selected from: having a probability of being expressed above a predetermined threshold, having a probability of being expressed that is above a threshold set adaptively to select a predetermined number of tumour-specific mutations with the highest probabilities of being expressed amongst the tumour-specific mutations for which a probability was determined, having a probability of being expressed that is above a threshold set adaptively to select a predetermined top percentile of tumour-specific mutations amongst the tumour-specific mutations for which a probability was determined, having a power to detect a mutation as expressed that is above a predetermined threshold and a number of RNA reads showing the tumour-specific mutation above a threshold number associated with the power to detect a mutation as being expressed, and having a power to detect a mutation as expressed that is below a predetermined threshold and a number of RNA reads showing the tumour-specific mutation below a threshold number associated with the power to detect a mutation as being expressed.

The method may further comprise determining whether one or more of the tumour-specific mutations is likely to be clonal in a tumour of the subject, and identifying whether one or more of the tumour-specific mutations is likely to give rise to a clonal neoantigen. A clonal neoantigen may be a tumour-specific mutation that satisfies at least a criterion selected from: having a probability of being clonal above a predetermined threshold, having a probability of being clonal that is above a threshold set adaptively to select a predetermined number of tumour-specific mutations with the highest probabilities of being clonal amongst the tumour-specific mutations for which a probability was determined, and having a probability of being clonal that is above a threshold set adaptively to select a predetermined top percentile of tumour-specific mutations amongst the tumour-specific mutations for which a probability was determined. Thus, the one or more predetermined criteria on whether the tumour-specific mutation is likely to be clonal may be selected from: the mutation having a likelihood of being clonal above a predetermined threshold, the mutation having a likelihood of being clonal that is above a threshold set adaptively to select a predetermined number of tumour-specific mutations with the highest likelihoods of being clonal amongst the tumour-specific mutations for which a likelihood was determined, and having a likelihood of being clonal that is above a threshold set adaptively to select a predetermined top percentile of tumour-specific mutations amongst the tumour-specific mutations for which a likelihood was determined.

A neoantigen may be a tumour-specific mutation that satisfies at least a criterion selected from: being predicted to result in a protein or peptide that is not expressed in the normal cells of the subject, being predicted to result in at least one peptide that is likely to be presented by an MHC molecule, being predicted to result in at least one peptide that is likely to be presented by an MHC allele that is known to be present in the subject, and being predicted to result in a protein or peptide that is immunogenic. For example, a neoantigen may be a tumour-specific mutation that satisfies a criterion that it is predicted to result in a change in the sequence of a protein (e.g. because it is coding, because it affects a splice site, because it results in a truncated peptide, etc.), thus resulting in a protein or peptide that may not be expressed in the normal cells of the subject. Whether or not this is the case may further be confirmed for example by comparison with a predicted normal proteome of the subject. Thus, the one or more criteria on whether the tumour-specific mutation is likely to give rise to a neoantigen may be selected from: the mutation being associated with an expression product that is expressed in tumour cells, the mutation being predicted to result in a protein or peptide that is not expressed in the normal cells of the subject, the mutation being predicted to result in at least one peptide that is likely to be presented by an MHC molecule, the mutation being predicted to result in at least one peptide that is likely to be presented by an MHC allele that is known to be present in the subject, and the mutation being predicted to result in a protein or peptide that is immunogenic.

The method may further comprise identifying one or more peptides associated with the one or more neoantigens (i.e. one or more peptide sequences that are predicted to be present in the tumour cells as a consequence of the presence of the tumour-specific mutation, where the tumour-specific mutation satisfies one or more criteria (such as e.g. criteria related to probability of being expressed, likelihood of giving rose to a neoantigen and/or likelihood of giving rise to a clonal neoantigen) as described above.

As the skilled person understands, the complexity of the operations described herein (due at least to the complexity of obtaining posterior probabilities requiring numerical integration as described herein, and the amount of data that is typically generated by sequencing genetic material) are such that they are beyond the reach of a mental activity. Thus, unless context indicates otherwise (e.g. where sample preparation or acquisition steps are described), all steps of the methods described herein are computer implemented.

According to a third aspect, there is provided a method of determining whether a tumour-specific mutation is likely to give rise to a neoantigen, the method comprising: determining whether the tumour-specific mutations is likely to be expressed in a tumour of the subject using the method of any embodiment of the first aspect; and determining whether the tumour-specific mutation satisfies one or more predetermined criteria applying to the result of the step of determining whether the tumour-specific mutation is likely to be expressed, and optionally one or more further criteria.

The method of the present aspect may have any one or more of the features of any preceding aspect.

According to a fourth aspect, there is provided a method of providing an immunotherapy for a subject that has been diagnosed as having cancer, the method comprising: identifying one or more neoantigens that are likely to be expressed in a tumour of the subject using a method as described herein, such as a method according to any embodiment of the first or second aspect, for example by identifying one or more tumour-specific mutations in the subject; determining whether one or more of the tumour-specific mutations is likely to be expressed in a tumour of the subject using the methods of any embodiment of the first aspect; selecting one or more tumour-specific mutations from the identified tumour-specific mutations based on the result of the determining and optionally one or more further criteria, such as e.g.as described in relation to the second aspect; and designing an immunotherapy that targets one or more of the neoantigens identified.

According to a further aspect, there is provided a method of treating a subject that has been diagnosed as having cancer, the method comprising: identifying one or more neoantigens by: identifying a plurality of tumour-specific mutations in the subject; determining whether one or more of the tumour-specific mutations is likely to be expressed in the subject; selecting one or more of the tumour-specific mutations as candidate neoantigens, wherein a candidate neoantigen is a tumour-specific mutation that satisfies at least one or more predetermined criteria on whether the tumour-specific mutation is likely to be expressed; and treating the subject with an immunotherapy that targets one or more of the selected candidate neoantigens. According to the present aspect, determining whether a tumour-specific mutation is likely to be expressed in a subject, comprises: obtaining, by a processor, RNA sequence data from one or more samples from the subject comprising tumour genetic material, the RNA sequence data comprising for each of the one or more samples: the number of reads in the sample that show the tumour-specific mutation (b), and the total number of reads at the location of the tumour-specific mutation (d), and determining, by the processor, the probabilities of observing the RNA sequence data if the tumour-specific mutation is (i) expressed and (ii) not expressed. The method may further comprise determining the posterior probability that the tumour-specific mutation is expressed depending on: a prior probability of the mutation being expressed, and the probabilities of observing the RNA sequence data if the tumour-specific mutation is (i) expressed and (ii) not expressed. The method may have any of the features described in relation to any preceding aspect.

The method may have any one or more of the following features.

The present disclosure also relates to immunotherapies that target one or more neoantigens associated with a tumour-specific mutation that has been determined to be expressed in a tumour using a method as described herein, and to methods for designing and/or providing such immunotherapies. According to any aspect described herein, an immunotherapy may be an immunogenic composition, a composition comprising immune cells or a therapeutic antibody. The immunogenic composition may comprise one or more neoantigens identified (such as e.g. a neoantigen peptide or protein or a cell displaying the neoantigen), or material sufficient for expression of the one or more neoantigens identified (e.g. a DNA or RNA molecule which encodes the neoantigen(s)). The composition comprising immune cells may comprise T cells, B cells and/or dendritic cells. The composition comprising a therapeutic antibody may comprise one or more antibodies that recognise at least one of the one or more of the neoantigens identified. An antibody may be a monoclonal antibody.

In any embodiment of any aspect, the cancer may be selected from bladder cancer, gastric cancer, oesophageal cancer, breast cancer, colorectal cancer, cervical cancer, ovarian cancer, endometrial cancer, kidney cancer (renal cell), lung cancer (small cell, non-small cell and mesothelioma), brain cancer (gliomas, astrocytomas, glioblastomas), melanoma, lymphoma, small bowel cancers (duodenal and jejunal), leukemia, pancreatic cancer, hepatobiliary tumours, germ cell cancers, prostate cancer, head and neck cancers, thyroid cancer and sarcomas. The cancer may be lung cancer. The cancer may be melanoma. The cancer may be bladder cancer. The cancer may be head and neck cancer. In any embodiment of any aspect, the subject may be human. Designing an immunotherapy that targets one or more neoantigens identified may comprise designing one or more candidate peptides for each of the one or more neoantigens targeted, each peptide comprising at least a portion of a neoantigen targeted. Designing or providing an immunotherapy may comprise obtaining the one or more candidate peptides. The method may further comprise testing the one or more candidate peptides for one or more properties. Testing may be performed in vitro or in silico. For example, the one or more peptides may be tested for immunogenicity, propensity to be displayed by MHC molecules (optionally by specific MHC molecule alleles, where the alleles may have been chosen depending on the MHC alleles expressed by the subject), ability to elicit proliferation of a population of immune cells, etc. The method may further comprise producing the immunotherapy. The method may further comprise obtaining a population of dendritic cells that has been pulsed with one or more of the candidate peptides. The immunotherapy may be a composition comprising T cells that recognise at least one of the one or more of the neoantigens identified. The composition may be enriched for T cells that target at least one of the one or more of the neoantigens identified. The method may comprise obtaining a population of T cells and expanding the population of T cells to increase the number or relative proportion of T cells that target at least one of the one or more of the neoantigens identified. The method may further comprise obtaining a T cell population. A T cell population may be isolated from the subject, for example from one or more tumour samples obtained from the subject, or from a peripheral blood sample or a sample from other tissues of the subject. The T cell population may comprise tumour infiltrating lymphocytes. T cells may be isolated using methods which are well known in the art. For example, T cells may be purified from single cell suspensions generated from samples on the basis of expression of CD3, CD4 or CD8. T cells may be enriched from samples by passage through a Ficoll-opaque gradient. The method may further comprise expanding the T cell population. For example, T cells may be expanded by ex vivo culture in conditions which are known to provide mitogenic stimuli for T cells. By way of example, the T cells may be cultured with cytokines such as IL-2 or with mitogenic antibodies such as anti-CD3 and/or CD28. The T cells may be co-cultured with antigen-presenting cells (APCs), which may have been irradiated. The APCs may be dendritic cells or B cells. The dendritic cells may have been pulsed with peptides containing one or more of the identified neoantigens as single stimulants or as pools of stimulating neoantigen peptides. Expansion of T cells may be performed using methods which are known in the art, including for example the use of artificial antigen presenting cells (aAPCs), which provide additional co-stimulatory signals, and autologous PBMCs which present appropriate peptides. Autologous PBMCs may be pulsed with peptides containing neoantigens as discussed herein as single stimulants, or alternatively as pools of stimulating neoantigens.

According to a further aspect, there is provided a method for expanding a T cell population for use in the treatment of cancer in a subject, the method comprising: identifying one or more neoantigens using a method as described herein, such as a method according to any embodiment of the second aspect; obtaining a T cell population comprising a T cell which is capable of specifically recognising one of the identified neoantigens; and co-culturing the T cell population with a composition comprising the identified neoantigens. The method may have one or more of the following features. The T cell population obtained may be assumed to comprise a T cell capable of specifically recognising one of the identified neoantigens. The method preferably comprises identifying a plurality of neoantigens. The neoantigens may be clonal neoantigens. The T cell population may comprise a plurality of T cells each of which is capable of specifically recognising one of the plurality of identified neoantigens, and co-culturing the T cell population with a composition comprising the plurality of identified neoantigens. The co-culture may result in expansion of the T cell population that specifically recognises the one or more neoantigens. The expansion may be performed by co-culture of a T cell with a neoantigen and an antigen presenting cell. The antigen presenting cell may be a dendritic cell. Thus, the expansion may be a selective expansion of T cells which are specific for the neoantigen. The expansion may further comprise one or more non-selective expansion steps.

According to a further aspect, there is provided a composition comprising a population of T cells obtained or obtainable by a method according to any embodiment of the preceding aspect. According to a further aspect, there is provided a composition comprising a neoantigen, neoantigen specific immune cell, or an antibody that recognises a neoantigen, for use in the treatment or prevention of cancer in a subject, wherein said neoantigen has been identified as a neoantigen (e.g. identified as being derived from a tumour-specific mutation that is expressed in a tumour of the subject), using the methods described herein. According to a further aspect, there is provided a composition comprising a neoantigen, neoantigen specific immune cell, or an antibody that recognises a neoantigen, wherein said neoantigen has been identified as a neoantigen (e.g. identified as being derived from a tumour-specific mutation that is expressed in a tumour of the subject) using the methods described herein. According to a further aspect, there is provided a cell or population of cells expressing a neoantigen on its surface, wherein said neoantigen has been identified as a neoantigen (e.g. identified as being derived from a tumour-specific mutation that is expressed in a tumour of the subject) using the methods described herein. According to a further aspect, there is provided a neoantigen, immune cell which recognises a neoantigen, or antibody which recognises a neoantigen, for use in the treatment or prevention of cancer in a subject, wherein said neoantigen has been identified as a neoantigen (e.g. identified as being derived from a tumour-specific mutation that is expressed in a tumour of the subject) using the methods described herein. According to a further aspect, there is provided a use of a neoantigen, immune cell which recognises a neoantigen, or antibody which recognises a neoantigen, in the manufacture of a medicament for use in the treatment or prevention of cancer in a subject, wherein said neoantigen has been identified as a neoantigen (e.g. identified as being derived from a tumour-specific mutation that is expressed in a tumour of the subject) using the methods described herein. According to a further aspect, there is provided a method of treating a subject that has been diagnosed as having cancer, the method comprising administering an immunotherapy that has been provided using the methods described herein, or a composition as described herein. According to a further aspect, there is provided a system comprising: a processor; and a computer readable medium comprising instructions that, when executed by the processor, cause the processor to perform the steps of any method described herein, such as a method according to any embodiment of the first, second, third or fourth aspects above.

According to a further aspect, there is provided one or more non-transitory computer readable media comprising instructions that, when executed by one or more processors, cause the one or more processors to perform the steps of any method described herein, such as a method according to any embodiment of the first, second, third or fourth aspects above.

According to a further aspect, there is provided a computer program comprising code which, when the code is executed on a computer, causes the computer to perform the steps of any method described herein, such as a method according to any embodiment of the first, second, third or fourth aspects above.

### BRIEF DESCRIPTION OF THE FIGURES

FIG. 1A illustrates schematically the problem of determining allele specific expression in a homogeneous diploid cell population and FIG. 1B illustrates schematically the problem of determining allele specific expression in a mixed sample comprising variant cells (e.g. tumour cells) and reference cells (e.g. healthy cells).

FIGS. 1C and 1D illustrate schematically the features of a model for determining the power to detect the expression of a mutated allele in a mixed sample. C. Variables of the model for detection of a mutated allele (here indicated by a “C”) in a mixed sample comprising a variant population of cells that have at least one copy of the allele (e.g. tumour cells, here illustrated as having a genotype G_{V} heterozygous for the mutated allele), and a reference population of cells that do not have any copies of the allele (e.g. healthy cells, comprising only reference alleles here illustrated as “T”, the healthy cells having a genotype G_{N} homozygous for T in the illustrated example). The sample comprise a proportion t of variant cells (also referred to as “tumour purity”). The expression data consists of a total number d of reads covering the locus, of which b reads show the variant allele. The set of genotypes in the population is denoted G={G_{V},G_{N}}. The variant cells express both the reference and the variant alleles, with proportion 0 (fraction of the expression in the tumour cells due to the reference allele), and the healthy cells express a proportion a of the total number of reads observed at the locus. These variables are used to determine the likelihood of observing any number of reads b with the variant allele under a null model of no expression of the variant allele (M₀) and under an alternative model where the variant allele is expressed (M₁). This is estimated in the illustrated example as binomial probability of sample b variant reads given that the total number of reads is d, and the probability of sampling a read with the mutation from the mixed sample is given by ξ. D. Likelihoods of the null and alternative models. The likelihood of the null model (no expression, P(b|M₀), also denoted as P(b,d|α,t,E=0)) decreases as the number of variant reads increases. The likelihood of the alternative model (variant is expressed, P(b|M₁), also denoted as P(b,d|α,t,E=1)) has a local maximum that depends on the parameters of the model. It is therefore possible to identify, using the likelihood of M₀, a critical value b_{c} that is such that the probability of rejecting the null model (which occurs when b>b_{c}) when the null model was correct (P(False positive)) is equal to a predetermined value (area under the curve of the likelihood of M₀ to the right of b_{c}). With this b_{c}, the probability of correctly identifying a variant that is indeed expressed is given by the area under the curve of the likelihood of M₁ to the right of b_{c}. This is the power of a statistical test to accept or reject M₀ based on the likelihoods of the read data under M₀ and M₁.

FIG. 1E illustrates how the models described in C and D can be used to determine the probability that a variant is expressed (P(E=1|b,d,α,t)). The 2 plots in the top left corner show the likelihoods of the null (M₀, E=0) and alternative models (M₁, E=1) as illustrated on D. The top right plot show the priors over the parameter θ for the null and variant models, and the distribution of variable E (whether the variant is expressed, E=1, or not,E=0) and the hyperprior over parameter ρ of the distribution of variable E, according to an example of the methods described.

FIG. 2A is a flowchart illustrating schematically a method of determining whether a tumour-specific mutation is likely to be clonal, and its use in identifying clonal neoantigens.

FIG. 2B is a flowchart illustrating schematically a method of providing an immunotherapy.

FIG. 3 shows an embodiment of a system for determining whether a tumour-specific mutation is likely to be clonal and/or for identifying clonal neoantigens and/or for providing an immunotherapy.

FIG. 4 illustrates schematically a model used to evaluate sequence data according to methods disclosed herein.

FIGS. 5A and 5B illustrate schematically models used to determine the probability that a variant is expressed according to methods disclosed herein, based on data from a single tumour sample (A) or a plurality of tumour samples (B).

FIG. 6 shows the behaviour of the posterior probability of a variant being expressed (y-axis) as a function of the relative likelihood of models assuming that the variant is vs. is not expressed (r, x-axis). The plot shows a curve for each of a plurality of the parameter μρ (mean of the Beta distribution over ρ used as a hyperprior for ρ, where ρ is the parameter of a Bernoulli variable prior for E and E is a binary variable capturing whether the variant is expressed or not). The curves shown are for increasing values of μρ from the bottom curve (μ_{ρ}=0.1) to the top curve (μ_{ρ}=0.9).

FIG. 7 illustrates schematically how the fraction of total expression at a locus due to the normal cell population (a) can be estimated by fitting a line to data of total expression at the locus for samples with various purities (t). The plot illustrates relationships between total expression at the locus (total TPM) as a function of purity (t) for different values of α (from 0 to 1, clockwise from the top right quadrant), with TPM^{N}+TPM^{T}=5.

FIG. 8 shows the true negative and false positive (first bar of each subplot—variants that are known not to be expressed) and the false negative and true positive (second bar of each subplot, variants that are known to be expressed) rates obtained using the methods described herein to identify whether variants are expressed in datasets of known dilutions of a cell line with known variants with known expression status. Each column shows a different dilution and each row shows results using different sources for the genotypes and purity used in the model. The number of mutations that are in each category are overlaid on the bars. In each case, the smallest part of the bar is the erroneously assigned mutations (FP or FN mutations).

FIG. 9 shows boxplots of the power of detecting mutations estimated as described herein in the data of FIG. 8, separated by known expression status (top row=mutations that are known not to be expressed, bottom row-mutations that are known to be expressed). Each column shows a different dilution, and each subplot shows a distribution for the mutations that are not detected as expressed (left, i.e. TN on the top row, FN on the bottom row) and the mutations that are detected as not expressed (right). A. Ground truth genotypes and purity. B. Fixed genotypes, ground truth purity. C. Estimated genotypes, ground truth purity. D. Ground truth genotypes, estimated purity. E. Fixed genotypes, estimated purity.

FIG. 10 shows the true positive rate as a function of the power to detect expression in the data of FIGS. 8 and 9, binning the mutations by power. Each plot is for a model using different sources for the genotypes and purity.

FIG. 11 shows the true negative and false positive (first bar of each subplot—variants that are known not to be expressed) and the false negative and true positive (second bar of each subplot, variants that are known to be expressed) rates obtained using the methods described herein to identify whether variants are expressed in datasets of known dilutions of a cell line with known variants with known expression status, using an estimation of the value of the cell expression ratio estimated from data. Each column shows a different dilution and each row shows results using different sources for the genotypes and purity used in the model. The number of mutations that are in each category are overlaid on the bars. In each case, the smallest part of the bar is the erroneously assigned mutations (FP or FN mutations). The data is the same as that used in FIG. 8B.

FIG. 12 shows the distribution of estimated power to detect an expressed variant obtained using the methods described herein for the data on FIG. 11. Each subplot shows results using different sources for the genotypes and purity used in the model. A. Ground truth genotypes and purity. B. Fixed genotypes, ground truth purity. C. Estimated genotypes, ground truth purity. D. Ground truth genotypes, estimated purity. E. Fixed genotypes, estimated purity.

FIG. 13 shows the calculated (ground truth) true positive rate as a function of the estimated power for the data on FIGS. 11 and 12. Each row shows results using different sources for the genotypes and purity used in the model.

FIG. 14 shows the number of variants associated with values between 0 and 1 of the probability of a mutation being expressed as determined using the methods described herein for the data of FIGS. 11-13, colour coded by read count (read count=0 or not) and split by mutations that are truly not expressed (top row) and mutations that are truly expressed (bottom row). Each column shows results for a different purity (5%, 10% or 30%).

FIG. 15 shows the number of variants associated with values between 0 and 1 of the probability of a mutation being expressed as determined using the variant allele fraction (VAF) of the mutation in the read data, for the data of FIGS. 11-14. Only data points where the read count is not 0 are shown as the VAF cannot be calculated for variants with read count=0. The data is split by mutations that are truly not expressed (top row) and mutations that are truly expressed (bottom row). Each column shows results for a different purity (5%, 10% or 30%).

FIG. 16 shows the receiver operator characteristics (ROC) curve for the identification of variants as expressed or not expressed using the probabilities of a mutation being expressed as determined using the methods described herein (A) or the probability of a mutation being expressed as determined using the variant allele fraction of the mutation in the read data (B), for the data of FIGS. 11-15. ROC curves show the true positive rate as a function of the false positive rate for different values of the cutoff (illustrated as the colour of the curve, with the scale on the right hand side of the plot) on probability used to call a variant as expressed or not expressed. Data for the model using ground truth genotypes and purity, and a value of a estimated from data at different purities using a regression model are shown for A. This data only considers variants where the total number of reads was >0. The plots also indicate the area under the curve (AUC), the probability threshold (cutoff) that results in the highest true positive rate while keeping the false positive rate <0.05, and the corresponding false positive rate (FPR) and true positive rate (TPR).

FIG. 17 shows calibration curves for a model determining the probabilities of a mutation being expressed as determined using the methods described herein (A) or the probability of a mutation being expressed as determined using the variant allele fraction of the mutation in the read data (B), using the data of FIGS. 11-16. The calibration curves show the fraction of true positives (variants that are expressed) as a function of the probability bin (bins of 10% probability) obtained for each of the approaches in A and B. Data for the model using ground truth genotypes and purity, and a value of a estimated from data at different purities using a regression model are shown for A. This data only considers variants where the total number of reads was >0.

FIG. 18 shows receiver operator characteristics (ROC) curve for the identification of variants as expressed or not expressed using the probabilities of a mutation being expressed as determined using the methods described herein (A) or the probability of a mutation being expressed as determined using the variant allele fraction of the mutation in the read data (B), for the data of FIGS. 11-17. ROC curves show the true positive rate as a function of the false positive rate for different values of the cutoff (illustrated as the colour of the curve, with the scale on the right hand side of the plot) on probability used to call a variant as expressed or not expressed. Data for the model using ground truth genotypes and purity, and a value of a estimated from data at different purities using a regression model are shown for A. This figure shows the same information as on FIG. 16 but determined using data including cases where the total number of reads=0. The plots also indicate the area under the curve (AUC), the probability threshold (cutoff) that results in the highest true positive rate while keeping the false positive rate <0.05, and the corresponding false positive rate (FPR) and true positive rate (TPR).

FIG. 19 shows calibration curves for a model determining the probabilities of a mutation being expressed as determined using the methods described herein (A) or the probability of a mutation being expressed as determined using the variant allele fraction of the mutation in the read data (B), using the data of FIGS. 11-18. The calibration curves show the fraction of true positives (variants that are expressed) as a function of the probability bin (bins of 10% probability) obtained for each of the approaches in A and B. Data for the model using ground truth genotypes and purity, and a value of a estimated from data at different purities using a regression model are shown for A. This data only considers variants where the total number of reads was >0 as well as variants where the total number of reads was =0.

### DETAILED DESCRIPTION

While it is generally accepted that cancer antigens such as those resulting from cancer specific variants (also referred to as “tumour-specific mutations”) represent promising therapeutic targets provided that they are expressed by cancer cells (see e.g. Heemskerk, Kvitsborg & Schumacher, 2013), approaches to determine whether these antigens are expressed have been relatively crude. For example, it has been suggested to use whole genome or whole exome sequencing to identify mutations in genomic DNA, and then use RNA sequencing to identify genes that are expressed in tumour material, and focus all efforts on those (see e.g. Heemskerk, Kvitsborg & Schumacher, 2013). Similarly, it has been suggested that RNA sequencing data could be used to identify tumour-specific mutations, with the advantage that this is not limited to known genes and can identify e.g. intragenic fusions, novel transcripts, etc. However, this is complicated by factors such as the differing levels of mRNA expression of any transcript in tumour and normal tissues (making comparisons difficult), and the possibility that such an approach would not be suitable to identify variants in RNA species present a low level (see e.g. Heemskerk, Kvitsborg & Schumacher, 2013). A gene centric approach to RNA expression combines information from variant and normal transcripts of the gene (both of which may be present in the tumour cells). Additionally, because tumour samples are typically mixed samples comprising tumour and normal cells, this also provides an aggregate signal from healthy and tumour cells, altogether providing very uncertain information about whether a variant is in fact expressed. Thus, there is a need for a more sensitive approach that can take into account all of these factors to more confidently identify the presence of variants in RNA expression data. Approaches to determining allele-specific expression have been developed in the context of determining allelic imbalance. For example, Castel et al. (2015) suggested an approach combining quality control measures with a binomial test to determine whether the ratio of two germline alleles is significantly different from the expected 0.5. However, such approaches only determine whether two germline alleles expected to be present in a pure diploid heterozygous population are differentially expressed. They are not applicable to the much more complex problem of detecting allele-specific expression of tumour-specific mutations. The present disclosure provides methods that solve this problem.

In the present disclosure, the following terms will be employed, and are intended to be defined as indicated below.

The term “allele-specific expression” (also referred to as “allelic expression”) refers to the amount of mRNA that is transcribed from a particular allele, or to whether a particular allele is expressed (i.e. whether any mRNA is transcribed from the particular allele). In the context of the present disclosure, determining allele-specific expression refers to determining whether an allele is expressed unless indicated otherwise. Allele specific expression is typically a property of a particular cell, cell population, tissue, sample or individual. In the context of the present disclosure, the alleles of interest are alleles that are present in a tumour and not in a germline population. Thus, alleles of interest (i.e. for which expression is to be detected and/or quantified) may be referred to interchangeably as “mutations” or “mutated alleles” or “variant alleles”. A mutated allele may be a tumour specific mutation, and a reference allele may be the corresponding germline allele. A germline allele may also be referred to as a “normal” or “healthy” or “reference” allele. A pair of alleles may be referred to as a “major allele” and a “minor allele” in a population, where the major allele is that with the highest VAF and the minor allele is that with the lowest VAF for the locus (where in a population with only two alleles, VAFₘᵢₙₒᵣ+VAFₘₐⱼₒᵣ=1). Depending on the sample, such as e.g. the proportion of tumour cells in the sample and the cancer cell fraction of the mutation, the germline or the mutant allele may be the major allele.

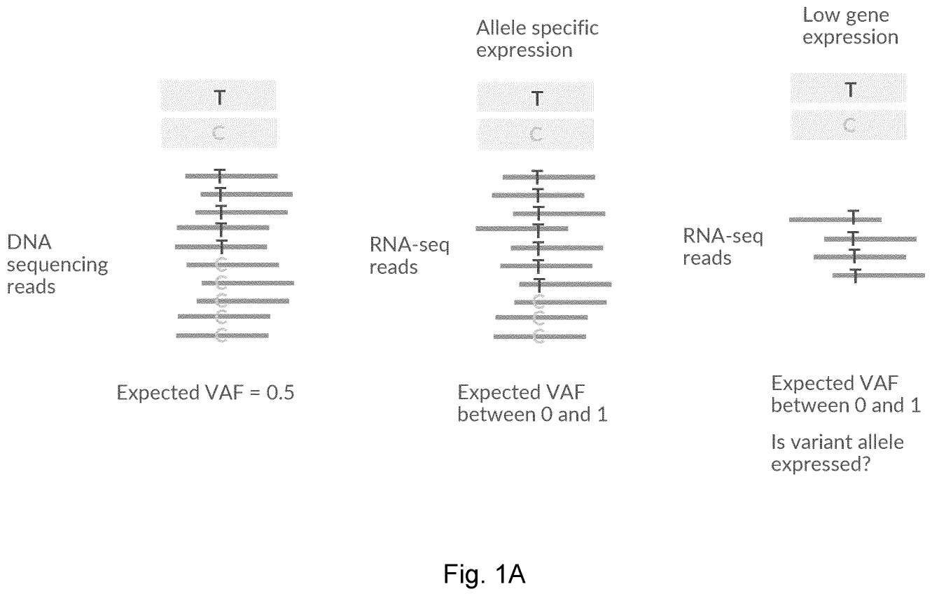

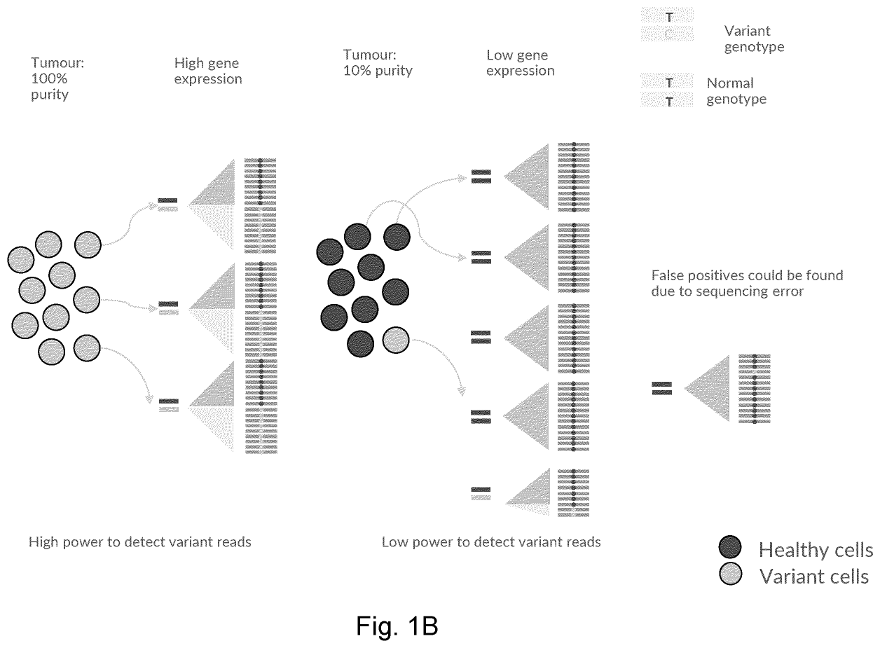

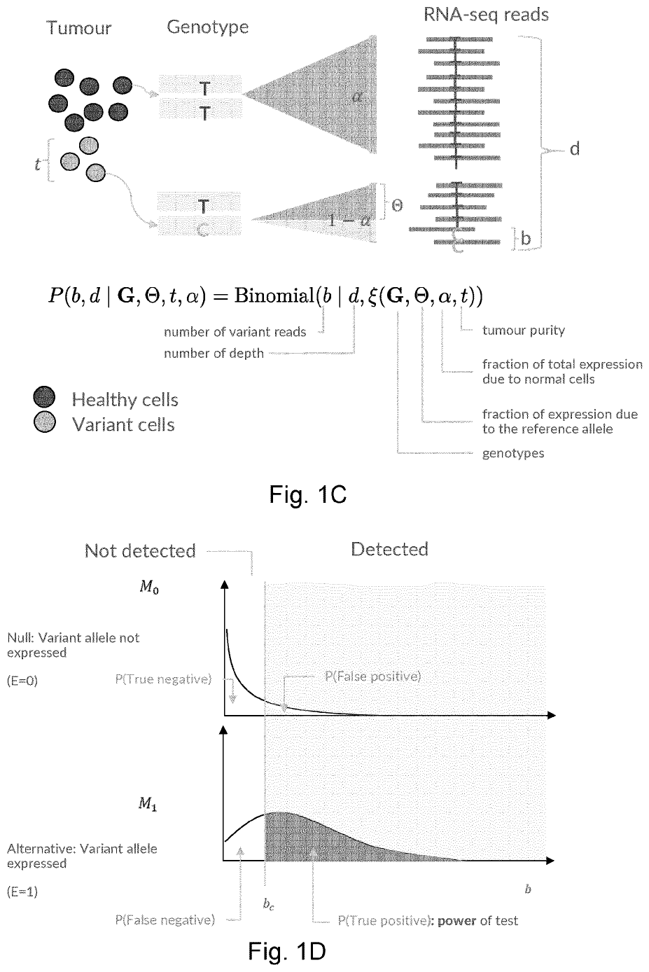

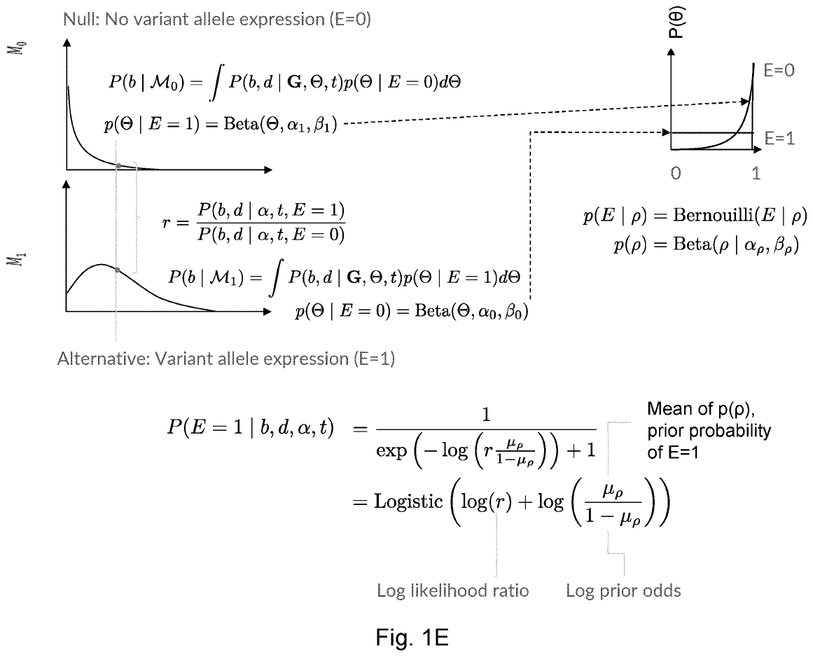

Determining allele-specific expression for a mutant allele in a mixed sample comprising mutant and germline cells is a considerably more complex problem than the determination of allelic imbalance. Allelic imbalance, which is also sometimes referred to in the prior art as “allelic expression”, quantifies expression variation between the two haplotypes of a diploid individual with heterozygous sites. This is conceptually different from the problem at hand (determining whether a variant is expressed) as statistical tests that assess departure from an expected 0.5 allele expression balance can be used (typically Binomial tests). Indeed, the methods described herein aim to determine whether a variant allele is present in expression data, rather than whether a variant and reference allele are expressed to different extents. FIG. 1A illustrates the problem of determining allele specific expression in a homogeneous diploid cell population. Additionally, allelic imbalance refers to a germline context where a single, pure population of heterozygous cells is considered. FIG. 1B illustrates the problem of determining allele specific expression in a mixed sample comprising variant cells and reference cells. This is a considerably more complex situation than when considering a homogeneous diploid cell population (as in the determination of allelic expression/allelic imbalance in the germline context). As illustrated in FIG. 1A, at the simplest level assuming a pure diploid heterozygous population (i.e. a population in which all cells have the same diploid heterozygous genotype), the genomic VAF is expected to be equal to 0.5, and genomic DNA reads should show an even representation of each allele. However, even in that same population, the number of RNA reads that represent each allele can be anywhere between 0 and 1 because the two alleles may not have the same expression level. In other words, any transcriptomic VAF between 0 and 1 could be plausible. Any sequencing process samples the population of molecules present in the sample. The sampling process results in a population of reads that is more or less likely to accurately represent the original population depending on the sampling depth, the amount of the molecules for a particular locus that are actually present in the sample (i.e. the expression level of the locus), and the sequencing error rate. Gene expression is known to have an extremely broad dynamic range, with some genes being abundantly expressed and others only being expressed at very low levels. At high levels of expression of a genomic locus, the number of reads for each allele should be sufficient to infer the expression VAF with relatively good confidence, particularly if neither allele has extremely low expression. However, at low levels of expression of the locus, the minor allele may not be sampled at all (simply by chance) and/or the number of reads that actually sample the minor allele may be so low that the presence of the variant cannot be distinguished from sequencing errors in view of the error rate of the sequencing platform used. This is even more complicated when looking at mixed populations, such as samples comprising tumour cells and non-tumour cells, as illustrated on FIG. 1B. Indeed, in such cases the number of genomic copies from which a variant allele (tumour specific allele) can be expressed in a tumour sample depends at least on the tumour purity (percentage of the cells in the sample that are tumour cells) and the copy number of the variant allele in the tumour population. Furthermore, in addition to different levels of expression of the variant and reference alleles in each cell, the expression level of the locus may differ between the tumour and non-tumour cells.

The present inventors have developed a method to determine the power to detect the expression of a mutated allele in a mixed sample comprising a variant population of cells that have at least one copy of the allele (e.g. tumour cells), and a reference population of cells that do not have any copies of the allele (e.g. normal cells). The method evaluates the likelihood of observing any number of reads b with the variant allele under a null model of no expression of the variant allele and under an alternative model where the variant allele is expressed. A statistical test to decide whether a variant is expressed is associated with 4 outcomes: a false positive (FP, the variant is identified as expressed when it is in fact not expressed), a false negative (FN, the variant is identified as not expressed when it is in fact expressed), a true positive (TP, a variant that is in fact expressed is identified as expressed), and a true negative (TN, a variant that is in fact not expressed is identified as not expressed). There is a trade-off between false positives and false negatives, as more permissive tests will capture more true positives but also more false positives. Thus, the FP rate can be fixed at an acceptable level (e.g. 5%), and the power (the probability of avoiding false negatives) can be calculated based on the FN rate at that chosen FP rate. As illustrated on FIG. 1D, the two likelihood values (under the null Model M₀ at the top and under the alternative model M₁ at the bottom) assume a Binomial distribution for the number of variant reads observed under each hypothesis, and show a different behaviour as a function of the number of variant reads b. It is possible to identify a critical number of variant reads b_{c} above which the null model is rejected (if number of variant reads b≥b_{c}, M₀ rejected) that is associated with any chosen false positive (FP) rate (FPR, probability of a false positive, i.e. a variant being called expressed when it is in fact not expressed). For example, a suitable FPR choice may be FPR=0.05. Thus, b, is the number of reads such that the P(b≥b_{c}) under M₀=0.05. With this threshold, any variant b≥b_{c} will be deemed to be expressed, and the probability of detecting a variant that is truly expressed (P(b≥b_{c}) under M₁) is the true positive (TP) rate (TPR), also referred to as power of the test. As illustrated on FIG. 1C and as explained further below, the likelihoods M₁ and M₀ are estimated based on e probability of observing the sequence data comprising a total number of reads covering the locus (d), a number of reads covering the locus and having the mutation (b), and taking into account the tumour purity (t), the genotype of the variant and reference cells (G, also denoted G_{V} and G_{N}, respectively for the variant and reference cell populations), the fraction of expression at the locus that is due to the reference allele (θ, reference read count/total read count), and the fraction of total expression (both variant and reference) due to the reference cells (α). The model described in detail below assumes that the variant and reference cell populations are genetically homogeneous at the locus (i.e. there is only one genotype G_{V} and one genotype G_{N}). However, the same principle can be applied in cases where the variant population comprises a population with the variant and a population without the variant (but that may not have the same genotype at the locus as the reference population). Thus, in the default implementation, the mutations are assumed to be ubiquitous in the sample(s) that is/are being analysed. The variable is the probability of sampling a read with the mutation from a mixed sample comprising variant cells with genotype G_{V} and reference cells with genotype G_{N}, where the variant cells represent a proportion t of the population. This is proportional to the number of copies of the variant allele in the mixed population, and its relative expression in the population. In the examples provided below, the probability of observing a number of variant reads b is assumed to follow a Binomial distribution conditional on the total number of read at the local and the value of the variable ξ. The likelihood of the null and alternative models is obtained using Bayes rule and different prior probabilities over the parameter θ (P(θ)) for the null and variant model. For example, in the null model the prior over θ may place higher densities around 0=1(most of the expression is expected to come from the reference allele) than around any other values (illustrated on FIG. 1E, top right), while in the alternative model the prior over θ gives a broad range of possible values of θ between 0 and 1 (illustrated as a flat prior on FIG. 22, top right). As illustrated on FIG. 1E, the likelihoods of M₀ and M₁ (and in particular their ratio r) can be used to estimate the posterior probability that a mutation is expressed, given expression data (P(E=1|b,d,α,t)). The probabilities p(θ|E=1) and p(θ|E=0) illustrated in the top right corner on FIG. 1E are the prior on θ (the reference ratio, which is the balance between reference allele expression and total expression at the locus), which are conditional on E (whether the variant is expressed or not). In the examples below, prior distributions that place a high amount of probability mass at 1 are used for E=0 (no expression, all the expression is expected to come from the reference allele), and relatively flat priors (similar amounts of mass for all values of θ) are used for E=1 (the variant allele may be expressed at any level). Assuming that E is a Bernouilli variable with parameter ρ, where ρ represents the prior knowledge of expression, the probability p(ρ) is a hyperprior on ρ. Using data b,d, and estimates of t (and optionally α), it is possible to calculate the joint posterior distribution over E and ρ, from which the marginal distribution on E can be calculated as shown on FIG. 1E by integrating over ρ, with r the likelihood ratio between the two models and μρ the mean of the Beta distribution over ρ (hyperprior).

The output of the methods may be used in a number of ways. For example, when the power to detect expression of a particular variant is determined to be low and/or the probability that the variant is expressed is determined to be low, the variant may not be excluded from a list of potentially immunogenic variants despite there being low evidence for expression of the variant. This information may be combined with one or more further criteria such as likelihood of binding to one or more MHC alleles of a peptide derived from the variant, likelihood of presentation by one or more MHC alleles of a peptide derived from the variant, likelihood of processing of a peptide derived from the variant, likelihood of immunogenicity of a peptide derived from the variant, differential binding affinity and/or likelihood of presentation between a peptide derived from the variant and the corresponding germline peptide, etc. Conversely, a variant that is identified as being associated with a high power to detect expression and/or a low probability of being expressed may be excluded from a list of potentially immunogenic variants if there is low evidence for expression of the variant. Further, the methods described herein can be applied separately or jointly for a plurality of tumour samples from the same subject, in order to determine whether a variant is likely to be present/expressed in all of the plurality of samples. This can provide an indication of whether the variant is likely present (and expressed) in all cells of a cancer. This may be referred to as a ubiquitous expressed variant. This may be assumed to be clonal (although true clonality cannot be established with certainty as this would strictly speaking require single cell sequence data for all or at least a representative proportion of cells in the cancer).

A “sample” as used herein may be a cell or tissue sample, a biological fluid, an extract (e.g. a DNA extract obtained from the subject), from which genomic and/or transcriptomic material can be obtained for genomic and/or transcriptomic analysis, such as genomic sequencing (e.g. whole genome sequencing, whole exome sequencing) or RNA sequencing (also referred to as “RNAseq” or “RNA-seq”). The sample may be a cell, tissue or biological fluid sample obtained from a subject (e.g. a biopsy). Such samples may be referred to as “subject samples”.

In particular, the sample may be a blood sample, or a tumour sample, or a sample derived therefrom. For the purposes of obtaining RNA sequence data, a “sample” as used herein may be a cell or tissue sample, or an extract (e.g. a RNA extract obtained from a subject) from which transcriptomic material can be obtained. For the purposes of obtaining DNA/genomic sequence data, a “sample” as used herein may be a cell or tissue sample, a biological fluid, an extract (e.g. a DNA extract obtained from the subject), from which genomic material can be obtained for genomic analysis, such as genomic sequencing (e.g. whole genome sequencing, whole exome sequencing). The sample may be one which has been freshly obtained from a subject or may be one which has been processed and/or stored prior to genomic/transcriptomic analysis (e.g. frozen, fixed or subjected to one or more purification, enrichment or extraction steps). The sample may be a cell or tissue culture sample. As such, a sample as described herein may refer to any type of sample comprising cells or genomic and/or transcriptomic material derived therefrom, whether from a biological sample obtained from a subject, or from a sample obtained from e.g. a cell line. In embodiments, the sample is a sample obtained from a subject, such as a human subject. The sample is preferably from a mammalian (such as e.g. a mammalian cell sample or a sample from a mammalian subject, such as a cat, dog, horse, donkey, sheep, pig, goat, cow, mouse, rat, rabbit or guinea pig), preferably from a human (such as e.g. a human cell sample or a sample from a human subject). Further, the sample may be transported and/or stored, and collection may take place at a location remote from the sequence data acquisition (e.g. sequencing) location, and/or any computer-implemented method steps described herein may take place at a location remote from the sample collection location and/or remote from the sequence data acquisition (e.g. sequencing) location (e.g. the computer-implemented method steps may be performed by means of a networked computer, such as by means of a “cloud” provider).

A “mixed sample” refers to a sample that is assumed to comprise multiple cell types or genetic material derived from multiple cell types. Within the context of the present disclosure, a mixed sample is typically one that comprises tumour cells or is assumed (expected) to comprise tumour cells, or genetic material derived from tumour cells. Genetic material can comprise genomic material (e.g. DNA) or transcriptomic material (e.g. RNA). Samples obtained from subjects, such as e.g. tumour samples, are typically mixed samples (unless they are subject to one or more purification and/or separation steps). Typically, the sample comprises tumour cells and at least one other cell type (and/or genetic material derived therefrom). For example, the mixed sample may be a tumour sample. A “tumour sample” refers to a sample derived from or obtained from a tumour. Such samples may comprise tumour cells and normal (non-tumour) cells. The normal cells may comprise immune cells (such as e.g. lymphocytes), and/or other normal (non-tumour) cells. The lymphocytes in such mixed samples may be referred to as “tumour-infiltrating lymphocytes” (TIL). A tumour may be a solid tumour or a non-solid or haematological tumour. A tumour sample may be a primary tumour sample, tumour-associated lymph node sample, or a sample from a metastatic site from the subject. A sample comprising tumour cells or genetic material derived from tumour cells may be a bodily fluid sample. Thus, the genetic material derived from tumour cells may be circulating tumour DNA or tumour DNA in exosomes. Instead or in addition to this, the sample may comprise circulating tumour cells. A mixed sample may be a sample of cells, tissue or bodily fluid that has been processed to extract genetic material. Methods for extracting genetic material from biological samples are known in the art. A mixed sample may have been subject to one or more processing steps that may modify the proportion of the multiple cell types or genetic material derived from the multiple cell types in the sample. For example, a mixed sample comprising tumour cells may have been processed to enrich the sample in tumour cells. Thus, a sample of purified tumour cells may be referred to as a “mixed sample” on the basis that small amounts of other types of cells may be present, even if the sample may be assumed, for a particular purpose, to be pure (i.e. to have a tumour fraction of 1 or 100%).

The term “tumour fraction” (also sometimes referred to as “tumour purity” or simply “purity”, or aberrant cell fraction (ACF)) refers to the proportion of DNA containing cells within a mixed sample that are tumour cells, or to the equivalent proportion that is assumed to result in a particular mixture of genetic material from tumour and non-tumour cells in a sample. Methods for determining the tumour fraction in a sample are known in the art. For example, in the context of cell or tissue samples, a tumour fraction may be estimated by analysing pathology slides (e.g. hematoxylin and eosin (H&E)-stained slides or other histochemistry or immunohistochemistry slides, by counting tumour cells in one or more representative areas of a sample), or using high throughput assays such as flow cytometry. In the context of samples comprising genomic material, a tumour fraction may be estimated using sequence analysis processes that attempt to deconvolute tumour and germline genomes such as e.g. ASCAT (Van Loo et al., 2010), ABSOLUTE (Carter et al., 2012), or ichorCNA (Adalsteinsson et al., 2017).

A “normal sample”, “healthy sample” or “germline sample” refers to a sample that is assumed not to comprise tumour cells or genetic material derived from tumour cells. A germline sample may be a blood sample, a tissue sample, or a purified sample such as a sample of peripheral blood mononuclear cells from a subject. Similarly, the terms “normal”, “germline” or “wild type” when referring to sequences or genotypes refer to the sequence/genotype of cells other than tumour cells. A germline sample may comprise a small proportion of tumour cells or genetic material derived therefrom, and may nevertheless be assumed, for practical purposes, not to comprise said cells or genetic material. In other words, all cells or genetic material may be assumed to be normal and/or sequence data that is not compatible with the assumption may be ignored.

The term “sequence data” refers to information that is indicative of the presence and preferably also the amount of genetic material in a sample that has a particular sequence. Such information may be obtained using sequencing technologies, such as e.g. next generation sequencing (NGS), for example whole exome sequencing (WES), whole genome sequencing (WGS), RNA sequencing or sequencing of captured genomic loci (targeted or panel sequencing), or using array technologies, such as e.g. copy number variation arrays, expression arrays or other molecular counting assays. When NGS technologies are used, the sequence data may comprise a count of the number of sequencing reads that have a particular sequence. When non-digital technologies are used such as array technology, the sequence data may comprise a signal (e.g. an intensity value) that is indicative of the number of sequences in the sample that have a particular sequence, for example by comparison to an appropriate control. Sequence data may be mapped to a reference sequence, for example a reference genome or transcriptome, using methods known in the art (such as e.g. Bowtie (Langmead et al., 2009)). Thus, counts of sequencing reads or equivalent non-digital signals may be associated with a particular genomic location (where the “genomic location” refers to a location in the reference genome to which the sequence data was mapped). Further, a genomic location may contain a mutation, in which case counts of sequencing reads or equivalent non-digital signals may be associated with each of the possible variants (also referred to as “alleles”) at the particular genomic location. The process of identifying the presence of a mutation at a particular location in a sample is referred to as “variant calling” and can be performed using methods known in the art (such as e.g. the GATK HaplotypeCaller,gatk.broadinstitute.org/hc/en-us/articles/360037225632-HaplotypeCaller). For example, sequence data may comprise a count of the number of reads (or an equivalent non-digital signal) which match a germline (also sometimes referred to as “reference”) allele at a particular genomic location, and a count of the number of reads (or an equivalent non-digital signal) which match a mutated (also sometimes referred to as “alternate”) allele at the genomic location.

Further, genomic sequence data may be used to infer copy number profiles along a genome, using methods known in the art. Copy number profiles refer to the number of genomic copies of genomic regions. Copy number profiles may be allele specific. In the context of the present disclosure, copy number profiles are preferably allele specific and tumour/normal sample specific. In other words, the copy number profiles used in the present disclosure are preferably obtained using methods designed to analyse samples comprising a mixture of tumour and normal cells, and to produce allele-specific copy number profiles for the tumour cells and the normal cells in a sample. Allele specific copy number profiles for mixed samples may be obtained from sequence data (e.g. using read counts as described above), using e.g. ASCAT (Van Loo et al., 2010). Other methods are known and equally suitable. Preferably, within the context of the present disclosure, the method used to obtain allele-specific copy number profiles is one that reports a plurality of possible copy number solutions and an associated quality/confidence metric. For example, ASCAT outputs a goodness-of-fit metric for each combination of values of ploidy (ploidy for a whole tumour sample, not segment-specific) and purity for which a corresponding allele-specific copy number profile was evaluated. Note that the tumour-specific copy number profiles generated by such methods represent an average or summary of the entire tumour cell population.

The term “total copy number” refers to the total number of copies of a genomic region in a sample. The term “major copy number” refers to the number of genomic copies of the most prevalent allele in a sample. Conversely, the term “minor copy number” refers to the number of genomic copies of the allele other than the most prevalent allele in a sample. Unless indicated otherwise, these terms refer to the inferred major and major copy numbers (and total copy numbers) for an inferred tumour copy number profile. The term “normal copy number” or “normal total copy number” refers to the number of copies of a genomic region in the normal cells in a sample. Normal cells typically have two copies of each chromosome (unless the cell is genetically male and the chromosome is a sex chromosome), and hence the normal copy number may in embodiments be assumed to be equal to 2 (unless the genomic region is on the X or Y chromosome and the sample under analysis is from a male subject, in which case the normal copy number may be assumed to be equal to 1). Alternatively, the normal copy number for a particular genomic region may be determined using a normal sample.

The term “log R value” (sometimes referred to as “log R”, “log RR”, “LLR”) refers to a measure of normalised total signal intensity, quantifying the total genomic copy number at a genomic locus. In the context of the present disclosure, the term typically refers to the log R value for a sample comprising tumour genetic material, and the normalisation is typically performed by reference to a normal sample (which is preferably a matched normal sample but may also be a process-matched normal sample or other suitable normal reference sample). For example, where NGS is used, the log R may be obtained as the normalised log transform of read depth (log(read depth tumour/read depth normal)). The term “mean B allele frequency” (MBAF, also sometimes referred to as “B allele frequency” (BAF)) is a measure of normalised allelic intensity ratio at a genomic location. In the context of the present disclosure, the term typically refers to the BAF value for a sample comprising tumour genetic material, and the normalisation is typically performed by reference to a normal sample (which is preferably a matched normal sample but may also be a process-matched normal sample or other suitable normal reference sample). For example, the BAF may be obtained as the ratio of the allele frequency for the tumour allele vs the normal allele. Copy number profiles typically comprise copy number estimates over genomic regions called “segments”. Thus, the BAF and log R associated with a genomic location may refer to the BAF and log R of the segment overlapping a particular genomic location (such as e.g. the genomic location of a mutation). Further, the BAF and log R can be used to obtain corresponding major and minor copy numbers. In embodiments, the values of copy number metrics may be provided for both a tumour copy number profile estimate and a normal copy number profile estimate, even if only the tumour copy number profile values are used.

The terms “tumour-specific mutation”, “tumour-specific variants”, “somatic mutation” or simply “mutation” or “variant” are used interchangeably and refer to a difference in a nucleotide sequence (e.g. DNA or RNA) in a tumour cell compared to a healthy cell from the same subject. The difference in the nucleotide sequence can result in the expression of a protein which is not expressed by a healthy cell from the same subject. For example, a mutation may be a single nucleotide variant (SNV), multiple nucleotide variant (MNV), a deletion mutation, an insertion mutation, a translocation, a missense mutation, a translocation, a fusion, a splice site mutation, or any other change in the genetic material of a tumour cell. A mutation may result in the expression of a protein or peptide that is not present in a healthy cell from the same subject. Mutations may be identified by exome sequencing, RNA-sequencing, whole genome sequencing and/or targeted gene panel sequencing and or routine Sanger sequencing of single genes, followed by sequence alignment and comparing the DNA and/or RNA sequence from a tumour sample to DNA and/or RNA from a reference sample or reference sequence (e.g. the germline DNA and/or RNA sequence, or a reference sequence from a database). Suitable methods are known in the art.

An “indel mutation” refers to an insertion and/or deletion of bases in a nucleotide sequence (e.g. DNA or RNA) of an organism. Typically, the indel mutation occurs in the DNA, preferably the genomic DNA, of an organism. In embodiments, the indel may be from 1 to 150 bases, for example 1 to 90, 1 to 50, 1 to 23 or 1 to 10 bases. An indel mutation may be a frameshift indel mutation. A frameshift indel mutation is a change in the reading frame of the nucleotide sequence caused by an insertion or deletion of one or more nucleotides. Such frameshift indel mutations may generate a novel open-reading frame which is typically highly distinct from the polypeptide encoded by the non-mutated DNA/RNA in a corresponding healthy cell in the subject.

A “neoantigen” (or “neo-antigen”) is an antigen that arises as a consequence of a mutation within a cancer cell. Thus, a neoantigen is not expressed (or expressed at a significantly lower level) by normal (i.e. non-tumour) cells. A neoantigen may be processed to generate distinct peptides which can be recognised by T cells when presented in the context of MHC molecules. As described herein, neoantigens may be used as the basis for cancer immunotherapies. References herein to “neoantigens” are intended to include also peptides derived from neoantigens. The term “neoantigen” as used herein is intended to encompass any part of a neoantigen that is immunogenic. An “antigenic” molecule as referred to herein is a molecule which itself, or a part thereof, is capable of stimulating an immune response, when presented to the immune system or immune cells in an appropriate manner. The binding of a neoantigen to a particular MHC molecule (encoded by a particular HLA allele) may be predicted using methods which are known in the art. Examples of methods for predicting MHC binding include those described by Lundegaard et al., O'Donnel et al., and Bullik-Sullivan et al. For example, MHC binding of neoantigens may be predicted using the netMHC-3 (Lundegaard et al.) and netMHCpan4 (Jurtz et al.) algorithms. A neoantigen that has been predicted to bind to a particular MHC molecule is thereby predicted to be presented by said MHC molecule on the cell surface.

A “clonal neoantigen” (also sometimes referred to as “truncal neoantigen”) is a neoantigen that results from a mutation that is present in essentially every tumour cell in one or more samples from a subject (or that can be assumed to be present in essentially every tumour cell from which the tumour genetic material in the sample(s) is derived). Similarly, a “clonal mutation” (sometimes referred to as “truncal mutation”) is a mutation that is present in essentially every tumour cell in one or more samples from a subject (or that can be assumed to be present in essentially every tumour cell from which the tumour genetic material in the sample(s) is derived). Thus, a clonal mutation may be a mutation that is present in every tumour cell in one or more samples from a subject. A “sub-clonal” neoantigen is a neoantigen that results from a mutation that is present in a subset or a proportion of cells in one or more tumour samples from a subject (or that can be assumed to be present in a subset of the tumour cells from which the tumour genetic material in the sample(s) is derived). Similarly, a “sub-clonal” mutation is a mutation that is present in a subset or a proportion of cells in one or more tumour samples from a subject (or that can be assumed to be present in a subset of the tumour cells from which the tumour genetic material in the sample(s) is derived). A neoantigen or mutation may be clonal in the context of one or more samples from a subject while not being truly clonal in the context of the entirety of the population of tumour cells that may be present in a subject (e.g. including all regions of a primary tumour and metastasis). Thus, a clonal mutation may be “truly clonal” in the sense that it is a mutation that is present in essentially every tumour cell (i.e. in all tumour cells) in the subject. This is because the one or more samples may not be representative of each and every subset of cells present in the subject. Thus, within the context of the present disclosure, a “clonal neoantigen” or “clonal mutation” may also be referred to as a “ubiquitous neoantigen” or “ubiquitous mutation”, to indicate that the neoantigen is present in essentially all tumour cells that have been analysed, but may not be present in all tumour cells that may exist in the subject. The terms “clonal” and “ubiquitous” are used interchangeably unless context indicates that reference to “true clonality” was intended. The wording “essentially every tumour cell” in relation to one or more samples or a subject may refer to at least 70%, at least 75%, at least 80%, at least 85%, at least 90%, at least 91%, at least 92%, at least 93%, at least 94% at least 95%, at least 96%, at least 97%, at least 98%, or at least 99% of the tumour cells in the one or more samples or the subject.

Nevertheless, a neoantigen/mutation that is identified as likely to be clonal (or “ubiquitous”) may be considered likely to be truly clonal, or at least more likely to be truly clonal than a neoantigen/mutation that is identified as unlikely to be clonal. Further, the confidence in the probability that a clonal neoantigen/mutation identified in a subject is truly clonal increases when the sample(s) used to identify the clonal neoantigen/mutation capture a more complete picture of the genetic diversity of the tumour (e.g. by including a plurality of samples from the subject, such as e.g. samples from different regions of the tumour, and/or by including samples that inherently capture a diversity of tumour cells such as e.g. ctDNA samples). Conversely, a neoantigen/mutation that is identified as unlikely to be clonal is unlikely to be truly clonal, because the identification that the neoantigen/mutation is unlikely to be clonal indicates that even in the restricted view afforded by the sampling process, there is evidence that the neoantigen/mutation is not present in all tumour cells. Thus, the process of identifying clonal neoantigens/mutations may be seen as prioritising which candidate neoantigens/mutations are most likely to be clonal, based on the restricted view of the clonal structure of the subject's tumour available from the one or more samples.

The term “cancer cell fraction” (or “CCF”) refers to the proportion of tumour cells that contain a mutation, such as e.g. a mutation that results in a particular neoantigen. A cancer cell fraction may be estimated based on one or more samples, and as such may not be equal to the true cancer cell fraction in the subject (as explained above). Nevertheless, the cancer cell fraction estimated based on one or more samples may provide a useful indication of the likely true cancer cell fraction. Further, as explained above, the accuracy of such an estimate may increase when the sample(s) used to estimate the cancer cell fraction capture a more complete picture of the genetic diversity of the tumour. Additional sources of noise and confounding factors in genomic data mean that a cancer cell fraction determined from one or more samples represents an estimate. As such, although a truly clonal mutation/neoantigen should have a CCF=1, in practice mutations/neoantigens that are more likely to be clonal are expected to be associated with a higher CCF estimate (which may not be equal to 1) than mutations that are less likely to be clonal, which are expected to be associated with a lower CCF estimate.

For example, a cancer cell fraction estimate may be obtained by integrating variant allele frequencies with copy numbers and purity estimates as described by Landau et al. (2013). Such a CCF estimate can also be used to identify mutations that are likely to be clonal. For example, a clonal mutation may be defined as a mutation which has an estimated cancer cell fraction (CCF) 0.75, such as a CCF 0.80, 0.85. 0.90, 0.95 or 1.0. A subclonal mutation may be defined as a mutation which has a CCF <0.95, 0.90, 0.85, 0.80, or 0.75. Further, a CCF estimate may be associated with (e.g. derived from) a distribution associating a probability with each of a plurality of possible values of CCF between 0 and 1, from which statistical estimates of confidence may be obtained. For example, a mutation may be defined as likely to be a clonal mutation if the 95% CCF confidence interval is >=0.75, i.e. the upper bound of the 95% confidence interval of the estimated CCF is greater than or equal to 0.75. In other words, a mutation may be defined as likely to be a clonal mutation if there is an interval of CCF with lower bound L and upper bound H that is such that P(L<CCF<H)=95% with H>=0.75. Alternatively, a mutation may be identified as clonal if there is more than a 50% chance or probability that its cancer cell fraction (CCF) reaches or exceeds the required value as defined above, for example 0.75 or 0.95, such as a chance or probability of 50%, 55%, 60%, 65%, 70%, 75%, 80%, 85%, 90%, 95% or more. In other words, a mutation may be identified as clonal if P(CCF>0.75)>=0.5. For example, mutations may be classified as likely clonal or subclonal based on whether the posterior probability that their CCF exceeds 0.95 (or 0.75, or any other chosen threshold) is greater or lesser than 0.5, respectively.

In this context, as will be explained further below, a mutation may be identified as likely to be clonal if P(CCF=1) exceeds a threshold. The threshold may be fixed. For example, a mutation may be identified as likely to be clonal if P(CCF=1)>0.05. Alternatively, the threshold may be determined for a particular set of mutations that are investigated. In embodiments, the threshold may be set based on a benchmarking data set with known clonal/non-clonal status, to reach a predetermined precision and/or recall. A benchmarking data set may be obtained using synthetic data and/or using a data set obtained from a population with known clonality structure (for example a cell line mixture data). For example, a mutation may be identified as likely clonal if P(CCF=1)>t where t is the maximum value that is such that 95% (or any other value such as e.g. 50%, 55%, 60%, 65%, 70%, 75%, 80%, 85%, 90%) of true clonal mutations in a benchmarking dataset are identified (i.e. a false negative rate of at most 5%). As another example, a mutation may be identified as likely clonal if P(CCF=1)>t where t is the minimum value that is such that at least 50% (or any other value such as e.g. 50%, 55%, 60%, 65%, 70%, 75%, 80%, 85%, 90%) of the mutations that exceed the threshold in a benchmarking dataset are true clonal mutations (i.e. a true positive rate of at least 50%). Alternatively, the threshold may be set such that any mutation (or a certain % of mutations) that is associated with an estimated CCF that has a confidence interval meeting the criteria described above (e.g. it is such that the upper bound of the 95% confidence interval of the estimated CCF is greater than or equal to 0.75) is selected as likely to be clonal. Alternatively, the threshold may be set such that any mutation (or a certain % of mutations) that is associated with an estimated CCF that has a posterior probability distribution meeting the criteria described above (e.g. a posterior probability that their CCF exceeds 0.95 (or 0.75, or any other chosen threshold) is greater than 0.5) is selected as likely to be clonal.

A cancer immunotherapy (or simply “immunotherapy”) refers to a therapeutic approach comprising administration of an immunogenic composition (e.g. a vaccine), a composition comprising immune cells, or an immunoactive drug, such as e.g. a therapeutic antibody, to a subject. The term “immunotherapy” may also refer to the therapeutic compositions themselves. In the context of the present disclosure, the immunotherapy typically targets a neoantigen. For example, an immunogenic composition or vaccine may comprise a neoantigen, neoantigen presenting cell or material necessary for the expression of the neoantigen. As another example, a composition comprising immune cells may comprise T and/or B cells that recognise a neoantigen. The immune cells may be isolated from tumours or other tissues (including but not limited to lymph node blood or ascites), expanded ex vivo or in vitro and re-administered to a subject (a process referred to as “adoptive cell therapy”). Instead or in addition to this, T cells can be isolated from a subject and engineered to target a neoantigen (e.g. by insertion of a chimeric antigen receptor that binds to the neoantigen) and re-administered to the subject. As another example, a therapeutic antibody may be an antibody which recognises a neoantigen. One skilled in the art will appreciate that if the neoantigen is a cell surface antigen, an antibody as referred to herein will recognise the neoantigen. Where the neoantigen is an intracellular antigen, the antibody will recognise the neoantigen peptide-MHC complex. As referred to herein, an antibody which “recognises” a neoantigen encompasses both of these possibilities. Further, an immunotherapy may target a plurality of neoantigens. For example, an immunogenic composition may comprise a plurality of neoantigens, cells presenting a plurality of neoantigens or the material necessary for the expression of the plurality of neoantigens. As another example, a composition may comprise immune cells that recognise a plurality of neoantigens. Similarly, a composition may comprise a plurality of immune cells that recognise the same neoantigen. As another example, a composition may comprise a plurality of therapeutic antibodies that recognise a plurality of neoantigens. Similarly, a composition may comprise a plurality of therapeutic antibodies that recognise the same neoantigen.

A composition as described herein may be a pharmaceutical composition which additionally comprises a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

References to “an immune cell” are intended to encompass cells of the immune system, for example T cells, NK cells, NKT cells, B cells and dendritic cells. In a preferred embodiment, the immune cell is a T cell. An immune cell that recognises a neoantigen may be an engineered T cell. A neoantigen specific T cell may express a chimeric antigen receptor (CAR) or a T cell receptor (TCR) which specifically binds a neoantigen or a neoantigen peptide, or an affinity-enhanced T cell receptor (TCR) which specifically binds a neoantigen or a neoantigen peptide (as discussed further hereinbelow). For example, the T cell may express a chimeric antigen receptor (CAR) or a T cell receptor (TCR) which specifically binds to a neo-antigen or a neo-antigen peptide (for example an affinity enhanced T cell receptor (TCR) which specifically binds to a neo-antigen or a neo-antigen peptide). Alternatively, a population of immune cells that recognise a neoantigen may be a population of T cell isolated from a subject with a tumour. For example, the T cell population may be generated from T cells in a sample isolated from the subject, such as e.g. a tumour sample, a peripheral blood sample or a sample from other tissues of the subject. The T cell population may be generated from a sample from the tumour in which the neoantigen is identified. In other words, the T cell population may be isolated from a sample derived from the tumour of a patient to be treated, where the neoantigen was also identified from a sample from said tumour. The T cell population may comprise tumour infiltrating lymphocytes (TIL).

The term “Antibody” (Ab) includes monoclonal antibodies, polyclonal antibodies, multispecific antibodies (e.g., bispecific antibodies), and antibody fragments that exhibit the desired biological activity. The term “immunoglobulin” (Ig) may be used interchangeably with “antibody”. Once a suitable neoantigen has been identified, for example by a method according to the disclosure, methods known in the art can be used to generate an antibody.

An “immunogenic composition” is a composition that is capable of inducing an immune response in a subject. The term is used interchangeably with the term “vaccine”. The immunogenic composition or vaccine described herein may lead to generation of an immune response in the subject. An “immune response” which may be generated may be humoral and/or cell-mediated immunity, for example the stimulation of antibody production, or the stimulation of cytotoxic or killer cells, which may recognise and destroy (or otherwise eliminate) cells expressing antigens corresponding to the antigens in the vaccine on their surface. The immunogenic composition may comprise one or more neoantigens, or the material necessary for the expression of one or more neoantigens. In addition, a neoantigen may be delivered in the form of a cell, such as an antigen presenting cell, for example a dendritic cell. The antigen presenting cell such as a dendritic cell may be pulsed or loaded with the neo-antigen or neo-antigen peptide or genetically modified (via DNA or RNA transfer) to express one, two or more neo-antigens or neoantigen peptides, for example 2, 3, 4, 5, 6, 7, 8, 9 or 10 neo-antigens or neo-antigen peptides. Methods of preparing dendritic cell immunogenic compositions or vaccines are known in the art.

Neoantigen peptides may be synthesised using methods which are known in the art. The term “peptide” is used in the normal sense to mean a series of residues, typically L-amino acids, connected one to the other typically by peptide bonds between the a-amino and carboxyl groups of adjacent amino acids. The term includes modified peptides and synthetic peptide analogues. The neoantigen peptide may comprise the cancer cell specific mutation (e.g the non-silent amino acid substitution encoded by a single nucleotide variant (SNV)) at any residue position within the peptide. By way of example, a peptide which is capable of binding to an MHC class I molecule is typically 7 to 13 amino acids in length. As such, the amino acid substitution may be present at position 1, 2, 3, 4, 5, 6, 7, 8, 9, 10, 11, 12 or 13 in a peptide comprising thirteen amino acids. In embodiments, longer peptides, for example 21-31-mers, may be used, and the mutation may be at any position, for example at the centre of the peptide, e.g. at positions 10, 11, 12, 13, 14, 15 or 16. Such peptides can also be used to stimulate both CD4 and CD8 cells to recognise neoantigens.

As used herein “treatment” refers to reducing, alleviating or eliminating one or more symptoms of the disease which is being treated, relative to the symptoms prior to treatment. “Prevention” (or prophylaxis) refers to delaying or preventing the onset of the symptoms of the disease. Prevention may be absolute (such that no disease occurs) or may be effective only in some individuals or for a limited amount of time.

As used herein, the terms “computer system” includes the hardware, software and data storage devices for embodying a system or carrying out a method according to the above described embodiments. For example, a computer system may comprise a central processing unit (CPU), input means, output means and data storage, which may be embodied as one or more connected computing devices. Preferably the computer system has a display or comprises a computing device that has a display to provide a visual output display (for example in the design of the business process). The data storage may comprise RAM, disk drives or other non-transitory computer readable media. The computer system may include a plurality of computing devices connected by a network and able to communicate with each other over that network. It is explicitly envisaged that computer system may consist of or comprise a cloud computer.

As used herein, the term “computer readable media” includes, without limitation, any non-transitory medium or media which can be read and accessed directly by a computer or computer system. The media can include, but are not limited to, magnetic storage media such as floppy discs, hard disc storage media and magnetic tape; optical storage media such as optical discs or CD-ROMs; electrical storage media such as memory, including RAM, ROM and flash memory; and hybrids and combinations of the above such as magnetic/optical storage media.

### Identification of Expressed Mutations

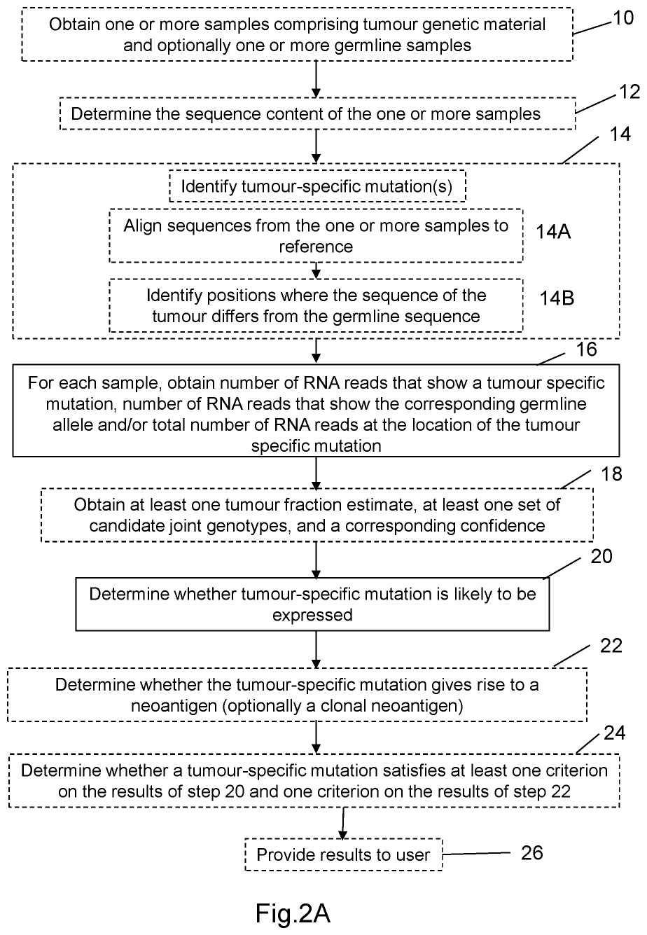

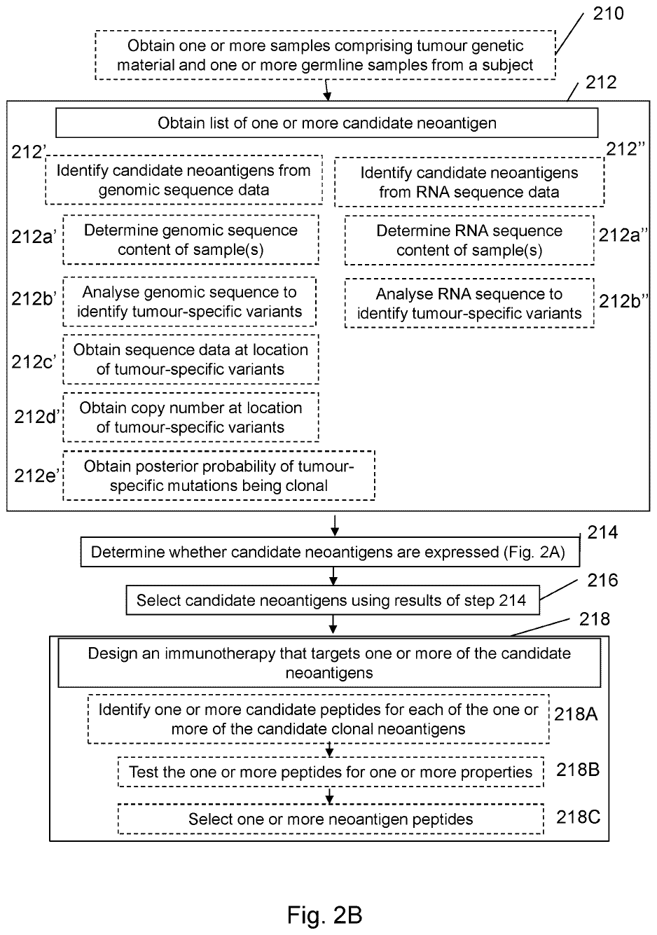

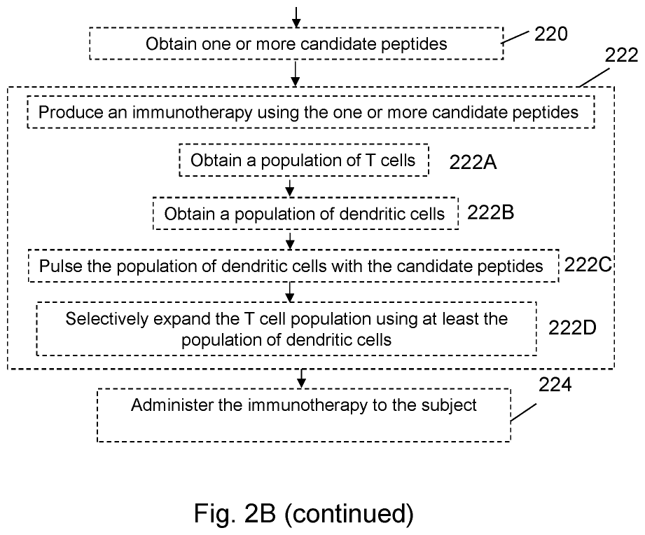

The present disclosure provides methods for determining whether a tumour-specific mutation is likely to be expressed using sequence data from one or more samples comprising tumour cells or genetic material derived therefrom. The disclosure also provides methods for identifying neoantigens comprising determining whether one or more tumour-specific mutations is/are likely to be expressed. An illustrative method will be described by reference to FIG. 2A. The method may comprise optional step 10 of obtaining one or more samples comprising tumour genetic material (such as e.g. one or more tumour samples). The sample(s) may be mixed samples comprising genomic material from multiple cell types including tumour cells and non-tumour cells (also referred to as “reference”, “healthy”, “normal” or “germline” cells). One or more germline samples may also be obtained, which do not comprise tumour genetic material. Germline samples may be matched germline samples, obtained from the same subject as the subject from which the one or more tumour samples are obtained. The use of a matched germline sample improves the accuracy of calling of somatic (tumour-specific) mutations, as any variant position identified in a tumour sample can be compared to variant positions in a matched germline sample to exclude germline variants. The same matched germline sample may be used to analyse a plurality of tumour samples from a subject. Further, the matched germline sample and one or more tumour samples may have been obtained at different times. For example, a first tumour sample and matched germline sample may have been obtained at the time of diagnosis or resection of a tumour, and a further tumour sample may be obtained and analysed together with the initial matched germline sample at a later time point. When a matched germline sample is not available, a reference sample or genome including common germline variants may be used. Alternatively, a process-matched normal sample may be used, which may not have been obtained from the same subject, or may have been obtained from a pool of subjects.

Optionally, the samples may be sequenced at step 12, to obtain at least RNA sequence data, and also optionally DNA sequence data. The RNA sequence data may be obtained by RNA sequencing. The DNA sequence data may be obtained using one of whole exome sequencing, or whole genome sequencing. Alternative methods such as e.g. allele-specific copy number arrays or expression arrays, may be used, although sequencing methods are preferred since they generate a digital output representative of the number of each particular sequence in a sample. Alternatively, the sequence data may have been previously obtained and may be received from a user interface, computing device or database, At optional step 14, the sequence data may be analysed to identify one or more mutations that are likely to be present in the tumour cells but not in non-cancerous cells. These represent tumour-specific mutations. These may be used as candidate neoantigens as explained further below. Step 14 may comprise the steps of aligning the sequences from the one or more samples (i.e. the mixed sample(s) and the germline sample(s), if available) to a reference such as e.g. a reference genome or transcriptome, at step 14A, and identifying genomic locations (also referred to as “positions” or “loci”) where the sequence of the tumour differs from the germline sequence or can be assumed to differ from the germline sequence (e.g. if a germline sequence for the subject is not available). This analysis may be performed using RNA sequence data, DNA sequence data, or both. For example, DNA sequence data may be used to identify tumour-specific mutations that are single nucleotide variants, multiple nucleotide variants or indels, and RNA sequence data may be used to identify tumour-specific mutations that are gene fusions or splicing variants. Thus, the tumour-specific mutations analysed may be somatic mutations present in a tumour of the subject from which the samples have been obtained. Any one or more of the tumour-specific mutations identified (or otherwise selected for example by a user through a user interface, or obtained from a computing device or database), may then be analysed to determine whether it is likely to be expressed. At step 16, sequence data is obtained for each sample comprising tumour genetic material, the data comprising the number of RNA reads in the sample that show the tumour-specific mutation (b) (these may also be referred to as read “containing” the tumour-specific mutation or reads “supporting” the tumour-specific mutation) and the total number of reads at the location of the tumour specific (d). Instead of these, the sequence data may comprise any two of: the number of RNA reads that show a tumour specific mutation, the number of RNA reads that show the corresponding germline allele, and the total number of RNA reads at the location of the tumour specific mutation (as all 3 numbers can be obtained from any two of these).

At optional step 18, information about at least one copy number solution compatible with each sample comprising tumour-genetic material, and about the tumour fraction in the sample (also referred to as “purity”) may be obtained. This information may comprise allele-specific copy number metrics for the tumour fraction of the sample selected from the major copy number, minor copy number, total copy number, mean B allele frequency, log R value and tumour ploidy, and the normal copy number, or information derived from these metrics such as a set of candidate joint genotypes that is compatible with these allele-specific copy number metrics. Not all such allele-specific copy number metrics are necessary as some contain redundant information and/or can be associated with suitable default values. For example, the normal copy number can be associated with a suitable default value (e.g. 2, assuming that the normal cells are diploid). Further, only two of the major copy number, total copy number and minor copy number are necessary to infer the third one. Similarly, those three values can be inferred from the MBAF and log R values (and vice versa). Optionally, a copy number solution may be associated with a corresponding confidence metric. When such a metric is not available, each copy number solution may be assumed to be equally likely. Each candidate joint genotype comprises a genotype at the location of the tumour-specific mutation for a normal population, and for a tumour cell population that comprises the tumour-specific mutation.

At step 20, it is determined whether a tumour-specific mutation is likely to be expressed, by determining the likelihoods of the sequence data (number of reads containing the tumour specific mutation and total number of reads at the locus of the tumour specific mutation) if the tumour-specific mutation is expressed and if the tumour specific mutation is not expressed. These likelihoods may depend on the probability of sampling a sequence read comprising the tumour-specific mutation from a sample if the tumour-specific mutation is expressed or not expressed, respectively, depending on the genotypes of the tumour and normal cell populations, the sequencing error rate, the tumour fraction of the sample and the fraction of total read counts for the gene comprising the tumour-specific mutation which is due to the normal cell population. The likelihoods may be compared to determine whether the tumour-specific mutation is likely to be expressed. This may comprise determining the posterior probability that the tumour-specific mutation is expressed (P(E=1|b,d,α,t)) depending on: a prior probability of the mutation being expressed (μ_{ρ}), and the likelihoods of the sequence data if the tumour-specific mutation is (i) expressed (P(b,d|α,t,E=1)) and (ii) not-expressed (P(b,d|α,t,E=0)). Alternatively, comparing the likelihoods may comprise determining whether the tumour specific mutation is expressed and determining the power to detect whether the tumour-specific mutation is expressed at a predetermined false positive rate. This may comprise determining a threshold number of reads (b_{c}) as the number of reads such that: the area under the curve of the likelihood of the number of reads that show the tumour specific mutation if the tumour-specific mutation is not expressed (P(b,d|α,t,E=0), P(b|E=0), P(b|M₀)) as a function of the number of RNA reads in the sample that show the tumour-specific mutation (b) is below the predetermined false positive rate. A tumour-specific mutation may be considered to be likely to be expressed if the number of reads showing the mutation is above this threshold number of reads. The power to detect whether the tumour-specific mutation is expressed may be the area under the curve of the likelihood of the number of reads that show the tumour specific mutation if the tumour-specific mutation is expressed (P(b,d|α,t,E=1), P(b|E=1), P(b|M₁)) as a function of the number of RNA reads in the sample that show the tumour-specific mutation (b), above the threshold number of reads (b_{c}). Step 20 may comprise determining that the tumour-specific mutation is likely to be expressed if the posterior probability is above a predetermined threshold. Step 20 may comprise determining that the tumour-specific mutation is likely to be expressed if the number of reads showing the mutation is above the threshold number of reads. Step 20 may comprise determining that the tumour-specific mutation is likely to be expressed if the number of reads showing the mutation is below the threshold number of reads but the power to detect whether the tumour specific mutation is expressed is below a predetermined threshold.

Step 20 may further comprise, prior to determining the likelihoods, a step of estimating the value of the fraction of total read counts for the gene comprising the tumour-specific mutation which is due to the normal cell population (a). This may comprise obtaining the total expression of the gene comprising the tumour specific mutation in a plurality of samples with different tumour purities, and fitting a regression model to the values of the total expression as a function of purity, and determining the value of a from the fitted regression model. This may be performed using equation (31) as explained further below. Step 20 may further comprise determining whether the total expression of the gene comprising the tumour-specific mutation is above a predetermined threshold. Step 20 may further comprise obtaining a prior probability that the tumour-specific mutation is expressed. The prior probability may be obtained from a user, computing device or database. The prior probability may be selected from a plurality of values depending on: (i) whether the total expression of the gene comprising the tumour-specific mutation is above a predetermined threshold, and (ii) whether the total number of reads at the location of the tumour specific mutaton is above a predetermine threshold. For example, when the total number of reads at the location of the tumour specific mutation is low (e.g. 0) but the total expression of the gene is not low (e.g. TPM≥1), the tumour-specific mutation may be considered likely to be expressed and the prior probability may be set to 0.5. As another example, when the total number of reads at the location of the tumour specific mutation is low (e.g. 0) and the total expression of the gene is low (e.g. TPM≤1), the tumour-specific mutation may be considered unlikely to be expressed (as the gene as a whole is not expressed) and the prior probability may be set to value below 0.5, such as e.g. 0.05. The total expression of the gene may be the total expression of the gene in the one or more samples, or in one or more other samples such as e.g. samples from the same or similar tumour types, or expression data derived thereof such as expression data from one or more databases (e.g. the Cancer Genome Atlas, TCGA—www.cancer.gov/about-nci/organization/ccg/research/structural-genomics/tcga).

At optional step 22, it is determined whether the tumour-specific mutation is likely to give rise to a neoantigen. For example, it may be determined whether the mutation is likely to result in a peptide or protein that is not expressed by a germline cell (whose genome does not contain the mutation). As another example, it may be determined whether the mutation is likely to be clonal in the tumour. This step may be performed at any point after step 14, and in particular need not be performed after steps 16-20. For example, candidate tumour-specific mutations may be filtered depending on whether they are likely to give rise to a neoantigen prior to determining whether the tumour-specific mutation is likely to be expressed. At step 24, tumour-specific mutations that satisfy one or more criteria that apply to the results of step 20 and one or more criteria that apply to the results of step 22 may be identified. These may be considered to represent candidate neoantigens, optionally candidate clonal neoantigens. At optional step 26, the results of any of the preceding steps (and in particular any of steps 20 to 24) may be provided to a user, for example through a user interface. These results may be used for example to provide an immunotherapy or prognosis for a subject, as will be described further below.

### Applications

The methods described herein may be used to determine whether a mutation is likely to be actually expressed, and whether a mutation is identified as unlikely to be expressed because the power to detect expression with the sequence data at hand is low. Further, the methods described herein may be used to detect mutations from RNA data (e.g. in particular for mutations that are only or advantageously detectable from RNA sequence data such as e.g. gene fusions and splice variants such as retained introns). Thus, also described herein are methods of detecting a candidate tumour-specific mutation, comprising determining whether the candidate tumour-specific mutation is likely to be expressed in a sample using the methods described herein. and the methods described herein may be used to determine the RNA sequencing depth that is necessary to be able to detect a particular mutation with a predetermined minimum power. For example, the approach may be used to determine the power to detect a mutation as being expressed given a plurality of candidate sequencing depths (resulting in corresponding b and d values). A sequencing depth that satisfies b>b_{c} at a significance level P(b≥b_{c}|M₀) and with a power above a predetermined value may be selected for use in sequencing a sample where a mutation is suspected to be present or expressed. Thus, also described herein are methods to determine a sequencing depth to be used to sequence RNA in a tumour sample, the method comprising; (i) determining whether a tumour-specific mutation is likely to be expressed in the sample if RNA sequence data is obtained from the sample at one or more candidate sequencing depths, by: simulating RNA sequence data comprising the number of RNA reads in the sample that show the tumour-specific mutation (b), and the total number of RNA reads at the location of the tumour-specific mutation (d) corresponding to the one or more sequencing depths if the tumour-specific mutation is truly expressed in the sample; and determining the likelihoods of the sequence data if the tumour-specific mutation is (i) expressed (P(b,d|α,t,E=1)) and (ii) not-expressed (P(b,d|α,t,E=0)), using the RNA sequence data, and (ii) selecting a sequence depth that is sufficient for the tumour specific mutation to be determined as likely to be expressed in the sample. In other words, also described herein is a method to determine a sequencing depth to be used to sequence RNA in a tumour sample, the method comprising performing the methods of determining whether a tumour-specific variant is likely to be expressed using RNA sequence data at one or more candidate sequencing depths, and using the results of the determining to select a candidate sequencing depth such that a tumour-specific variant that is truly expressed is determined as likely to be expressed according to the methods described herein.

The above methods find applications in the context of cancer diagnostic, prognostic and therapeutic approaches. In particular, the above methods may be used to provide immunotherapies that target neoantigens. Thus, also described herein are methods of providing an immunotherapy for a subject, the method comprising identifying one or more neoantigens from one or more samples from the subject.

FIG. 2B illustrates schematically an exemplary method of providing an immunotherapy. At optional step 210, one or more samples comprising tumour genetic material and optionally one or more germline samples are obtained from a subject. The subject may be a subject that has been diagnosed as having cancer, and may be (but does not need to be) the same subject for which the immunotherapy is provided. At step 212, a list of candidate neoantigens is obtained. This may comprise step 212′ of obtaining a list of candidate neoantigens from genomic sequence data and/or step 212″ of obtaining a list of candidate neoantigens from RNA sequence data. At optional step 212′, a list of candidate neoantigens may be obtained from genomic sequence data from the sample(s) using methods known in the art, for example as described in WO 2016/16174085, Landau et al. (2013), Lu et al. (2018), Leko et al. (2019), Hundal et al. (2019), and others. The list of candidate neoantigens may comprise a single neoantigen, or a plurality of neoantigens. Preferably, the list comprises a plurality of neoantigens. The neoantigens may be clonal neoantigens. Methods to identify clonal neoantigens are known in the art and include the methods described in WO 2016/16174085, Landau et al. (2013), Roth et al. (2014), McGranahan et al. (2016), and in co-pending application PCT/EP2022/058793. Instead or in addition to step 212′, one or more candidate neoantigens may be identified from RNA sequence data from the sample(s) at step 212″, for example by identifying one or more RNA sequence reads that include a variant. Variants can be identified by comparison with an expected healthy sequence such as a reference genome or transcriptome or RNA/DNA sequence from a normal sample of the subject. Thus, step 212″ may comprise optional step 212a″, where the RNA sequence content of the one or more mixed samples and optionally the matched sample may be determined, for example by sequencing the RNA (or mRNA) in the sample using RNA sequencing. Alternative methods such as e.g. expression arrays may be used, although sequencing methods are preferred since they generate a digital output representative of the number of each particular sequence in a sample. Step 212″ may further comprise optional step 212b″ of analysing the RNA sequence data to identify one or more mutations that are likely to be present in the tumour cells but not in non-cancerous cells. These represent tumour-specific mutations and may be used as candidate neoantigens. This may comprise the steps of aligning the RNA sequences from the one or more samples (i.e. the mixed sample(s) and the germline sample(s), if available). This may further comprise identifying locations where the RNA sequence of the tumour differs from the germline sequence or can be assumed to differ from the germline sequence (e.g. if a germline sequence for the subject is not available), for example based on a reference genome or transcriptome. Instead or in addition to this, step 212b″ may comprise aligning the RNA sequences from the one or more samples to a reference genome or transcriptome and identifying sequences that are not expected to be present in such a reference (e.g. novel transcripts, splicing variants, fusions, single nucleotide variants, indels). For example, a fusion or splicing variant may be identified if one or more reads align to non-contiguous sections of the reference transcriptome or genome, or fail to align to the reference genome or transcriptome.

Step 212′ may comprise optional step 212a′, where the sequence content of the one or more mixed samples and optionally the matched sample may be determined, for example by sequencing the genomic material in the sample using one of whole exome sequencing, or whole genome sequencing. Alternative methods such as e.g. allele-specific copy number arrays may be used, although sequencing methods are preferred since they generate a digital output representative of the number of each particular sequence in a sample. At optional step 212b′, the sequence data may be analysed to identify one or more mutations that are likely to be present in the tumour cells but not in non-cancerous cells. These represent tumour-specific mutations and may be used as candidate neoantigens. This may comprise the steps of aligning the sequences from the one or more samples (i.e. the mixed sample(s) and the germline sample(s), if available), and identifying genomic locations where the sequence of the tumour differs from the germline sequence or can be assumed to differ from the germline sequence (e.g. if a germline sequence for the subject is not available). At optional step 212c′, genomic sequence data for the mixed sample at the genomic location of a candidate tumour-specific mutation is obtained, comprising the count of reads supporting the mutated allele (also referred to as “non-reference allele”), the count of reads supporting the germline allele(s) (A, collectively referred to as “germline allele” if the locus is heterozygous in the germline population, also referred to as “reference”, “wild type” or “normal” allele) at the genomic location, and/or the total count of reads at the genomic location of the candidate tumour-specific mutation. Only two of these metrics need to be obtained as the third one can be deduced from any two of these. The sequence data may instead or in addition to this include read data or intensity data from which the counts can be obtained. At optional step 212d′, information about at least one copy number solution compatible with each sample comprising tumour-genetic material may be obtained. This information may comprise allele-specific copy number metrics for the tumour fraction of the sample selected from the major copy number, minor copy number, total copy number, mean B allele frequency (MBAF), log R value and tumour ploidy, and the normal copy number, or information derived from these metrics such as a set of candidate joint genotypes that is compatible with these allele-specific copy number metrics. Not all such allele-specific copy number metrics are necessary as some contain redundant information and/or can be associated with suitable default values. For example, the normal copy number can be associated with a suitable default value as explained above. Further, only two of the major copy number, total copy number and minor copy number are necessary to infer the third one. Similarly, those three values can be inferred from the MBAF and log R values (and vice versa). Optionally, a copy number solution may be associated with a corresponding confidence metric. When such a metric is not available, each copy number solution may be assumed to be equally likely. Each candidate joint genotype comprises a genotype at the location of the tumour-specific mutation for a normal population, a reference tumour population that does not comprise the tumour-specific mutation and a variant tumour cell population that comprises the tumour-specific mutation. At optional step 212e′, a posterior probability of a tumour-specific mutation being clonal is determined depending on: a prior probability of the mutation being clonal, and the probabilities of observing the sequence data if the tumour-specific mutation is (i) clonal and (ii) non-clonal, in view of a tumour fraction for each of the one or more samples and one or more candidate joint genotypes. Methods for obtaining such a posterior probability are further described below. A prior probability is a probability that represent a belief about a quantity before some evidence is taken into account. For example, a prior probability of a mutation being clonal may represent a probability of a mutation being clonal in the tumour, that is based on prior knowledge or assumptions, and does not take into account the sequence data from the mixed sample.

At step 214, it is determined whether the candidate neoantigens are likely to be expressed in a tumour of the patient, using a method as described in relation to FIG. 2A. At step 216, tumour-specific mutations that satisfy one or more criteria that apply to the results of step 214 and optionally one or more criteria are selected. For example, tumour-specific mutations that are associated with a probability of being expressed above a predetermined threshold may be selected. Alternatively, tumour-specific mutations that are associated with a probability of being expressed below a predetermined threshold may be excluded. As another example, tumour-specific mutations that are associated with a power to detect expression that is above a predetermined threshold, and a likelihood of a model assuming that the mutation is not expressed that is above a predetermined threshold may be excluded. As another example, tumour-specific mutations that are associated with a likelihood of a model assuming that the mutation is expressed that is below a predetermined threshold may be excluded unless they are also associated with a power to detect expression that is below a predetermined threshold.

One or more further criteria may be applied, which together with the determination of step 214 provide information as to whether the candidate neoantigens are likely to represent true neoantigens. For example, it may be determined whether the mutation is likely to result in a peptide or protein that is not expressed by a germline cell (whose genome does not contain the mutation). This step may be performed at any point after candidate neoantigens have been identified. One or more further criteria may be applied which provide information as to whether the candidate neoantigen is likely to be clonal in the tumour. For example, tumour-specific mutations associated with a probability of being clonal (such as e.g. as determined at step 212e′) that is above a predetermined threshold may be selected. Any of these criteria may be applied in any order. For example, candidate tumour-specific mutations may be filtered depending on whether they are likely to give rise to a neoantigen prior to determining whether the tumour-specific mutation is likely to be clonal, or vice-versa.

At step 218, an immunotherapy that targets at least one (and optionally a plurality) of the selected candidate neoantigens is designed. Designing such an immunotherapy may comprise identifying at step 218A one or more candidate peptides for each of the candidate clonal neoantigens. For example, a plurality of peptides may be designed for at least one of the candidate clonal neoantigens, which differ in their lengths and/or the location of a sequence variation that characterises the neoantigen compared to the corresponding germline peptide. At step 218B, the one or more peptides identified may be tested in vitro and or in silico to evaluate one or more properties such as their immunogenicity, likelihood of being displayed by a MHC molecule, etc. At optional step 218C, one or more of the peptides may be selected, for example based on the results of step 218B. At step 220, the selected peptides may be obtained. Peptides with selected sequences may be obtained using any method known in the art such as e.g. using an expression system or by direct synthesis. At step 222, an immunotherapy may be produced using the one or more candidate peptides. The immunotherapy may comprise the one or more candidate peptides or material sufficient for their expression (e.g. in the case of an immunogenic composition or vaccine), or may comprise molecules or cells that have been obtained using the candidate peptides (e.g. in the case of therapeutic antibodies that selectively bind the candidate peptides, or immune cells that specifically recognise the candidate peptides). At optional step 224, the immunotherapy may be administered to a subject, which is preferably the subject from which the samples used to identify the neoantigens have been obtained. An example of producing an immunotherapy comprising a T cell population selectively enriched with T cells that recognise one or more (optionally clonal) neoantigens will be described. At step 222A, a population of T cells may be obtained. The T cells may be obtained from the subject to be treated, but do not need to be. The T cells may be obtained from a tumour sample, from a blood sample, or from any other tissue sample. At step 222B, a population of dendritic cells may be obtained. For example, a population of dendritic cells may be derived from mononuclear cells (e.g. peripheral blood mononuclear cells, PBMCs) from the subject to be treated. At step 222C, the population of dendritic cells may be pulsed with the candidate peptides. At step 222D, the T cell population may be selectively expanded using the population of pulsed dendritic cells. Additional expansion factors such as e.g. cytokines or stimulating antibodies may be used.

Thus, the disclosure also provides a T cell composition comprising a T cell population selectively enriched with T cells that recognise one or more neoantigens that are likely to be expressed in a tumour, wherein the one or more neoantigens have been identified using any of the methods described herein. Neoantigens that are likely to be expressed may be neoantigens that are selected using the results of a method as described herein, such as e.g. as explained by reference to step 216 of FIG. 2B.

In a T cell composition as described herein the expanded population of neoantigen-reactive T cells may have a higher activity than the population of T cells which have not been expanded, as measured by the response of the T cell population to restimulation with a neoantigen peptide. Activity may be measured by cytokine production, and wherein a higher activity is a 5-10 fold or greater increase in activity.

References to a plurality of neoantigens may refer to a plurality of peptides or proteins each comprising a different tumour-specific mutation that gives rise to a neoantigen. Said plurality may be from 2 to 250, from 3 to 200, from 4 to 150, or from 5 to 100 tumour-specific mutations, for example from 5 to 75 or from 10 to 50 tumour-specific mutations. Each tumour-specific mutation may be represented by one or more neoantigen peptides. In other words, a plurality of neoantigens may comprise a plurality of different peptides, some of which comprise a sequence that includes the same tumour-specific mutation (for example at different positions within the sequence of the peptide, or within peptides of varying lengths). The tumour-specific mutations selected according to the methods described herein may be ones that are determined using a method as described herein to be likely to be expressed in a tumour of the patient to be treated. The tumour-specific mutations selected according to the methods described herein may be ones that are determined to be likely to be clonal in a tumour of the patient to be treated.

A T cell population that is produced in accordance with the present disclosure will have an increased number or proportion of T cells that target one or more neoantigens that are predicted to be expressed (and optionally clonal). That is to say, the composition of the T cell population will differ from that of a “native” T cell population (i.e. a population that has not undergone the expansion steps discussed herein), in that the percentage or proportion of T cells that target a neoantigen that is predicted to be expressed (and optionally clonal) will be increased. The T cell population according to the disclosure may have at least about 0.2, 0.3, 0.4, 0.5, 0 6, 0 7, 0 8, 0 9, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or 100% T cells that target a neoantigen that is predicted to be expressed.

The immunotherapies described herein may be used in the treatment of cancer. Thus, the disclosure also provides a method of treating cancer in a subject comprising administering an immunotherapeutic composition as described herein to the subject.

Suitably, in any embodiment of any aspect described herein, the cancer may be ovarian cancer, breast cancer, endometrial cancer, kidney cancer (renal cell), lung cancer (small cell, non-small cell and mesothelioma), brain cancer (gliomas, astrocytomas, glioblastomas), melanoma, merkel cell carcinoma, clear cell renal cell carcinoma (ccRCC), lymphoma, small bowel cancers (duodenal and jejunal), leukemia, pancreatic cancer, hepatobiliary tumours, germ cell cancers, prostate cancer, head and neck cancers, thyroid cancer and sarcomas. For example, the cancer may be lung cancer, such as lung adenocarcinoma or lung squamous-cell carcinoma. As another example, the cancer may be melanoma. In embodiments, the cancer may be selected from melanoma, merkel cell carcinoma, renal cancer, non-small cell lung cancer (NSCLC), urothelial carcinoma of the bladder (BLAC) and head and neck squamous cell carcinoma (HNSC) and microsatellite instability (MSI)-high cancers. In some embodiments, the cancer is non-small cell lung cancer (NSCLC). In other embodiments, the cancer is melanoma.

Treatment using the compositions and methods of the present disclosure may also encompass targeting circulating tumour cells and/or metastases derived from the tumour. The methods and uses for treating cancer described herein may be performed in combination with additional cancer therapies. In particular, the T cell compositions described herein may be administered in combination with immune checkpoint intervention, co-stimulatory antibodies, chemotherapy and/or radiotherapy, targeted therapy or monoclonal antibody therapy. ‘In combination’ may refer to administration of the additional therapy before, at the same time as or after administration of the T cell composition as described herein.

The disclosure also provides a method for producing an immunotherapeutic composition, the method comprising identifying a neoantigen as likely to be expressed and producing an immunotherapeutic composition that targets the neoantigen.

Also described herein is a method of treating a subject that has been diagnosed as having cancer, the method comprising: identifying one or more neoantigens by: identifying a plurality of tumour-specific mutations in the subject; determining whether one or more of the tumour-specific mutations is likely to be expressed in the subject; optionally determining whether one or more of the tumour-specific mutations is likely to be clonal in the subject; selecting one or more of the tumour-specific mutations as candidate neoantigens, wherein a candidate neoantigen is a tumour-specific mutation that satisfies at least one or more predetermined criteria on whether the tumour-specific mutation is likely to be expressed; and treating the subject with an immunotherapy that targets one or more of the selected candidate neoantigens; wherein determining whether a tumour-specific mutation is likely to be expressed in a subject is performed using the methods described herein.

In particular, determining whether a tumour-specific mutation is likely to be expressed in a subject may comprise: obtaining, by a processor, RNA sequence data from one or more samples from the subject comprising tumour genetic material, the sequence data comprising for each of the one or more samples, at least two of: the number of RNA sequence reads in the sample that show the tumour-specific mutation (b), the number of RNA sequence reads in the sample that show the corresponding germline allele, and the total number of RNA sequence reads at the location of the tumour-specific mutation (d), and determining, by the processor, the posterior probability that the tumour-specific mutation is expressed depending on: the mean of a prior probability of the mutation being expressed, and the likelihoods of the sequence data if the tumour-specific mutation is (i) expressed and (ii) not expressed, wherein the likelihoods of the sequence data are conditional on a tumour fraction for each of the one or more samples, and a fraction of the total expression at the locus of the tumour-specific mutation that is assumed to come from a normal population of cells that does not contain the tumour-specific mutation.

The method may further comprise determine whether a tumour-specific mutation is likely to be clonal in the subject by: obtaining, by a processor, genomic sequence data from one or more samples from the subject comprising tumour genetic material, the sequence data comprising for each of the one or more samples, at least two of: the number of reads in the sample that show the tumour-specific mutation (d_{b}), the number of reads in the sample that show the corresponding germline allele, and the total number of reads at the location of the tumour-specific mutation (d), and determining, by the processor, a posterior probability that the tumour-specific mutation is clonal depending on: a prior probability of the mutation being clonal, and the probabilities of observing the sequence data if the tumour-specific mutation is (i) clonal and (ii) non-clonal, in view of a tumour fraction for each of the one or more samples and one or more candidate joint genotypes each comprising a genotype at the location of the tumour-specific mutation for a normal population, a reference tumour population that does not comprise the tumour-specific mutation and a variant tumour cell population that comprises the tumour-specific mutation.

The candidate neoantigens may be selected as tumour-specific mutations that further satisfy at least one or more predetermined criteria on whether the tumour-specific mutation is likely to be clonal and/or to give rise to a neoantigen. The step of selecting, by said processor, one or more of the tumour-specific mutations as candidate neoantigens, may comprise determining whether the one or more tumour specific mutations satisfy one or more criteria on whether the tumour-specific mutation is likely to give rise to a neoantigen selected from: the mutation being predicted to result in a protein or peptide that is not expressed in the normal cells of the subject, the mutation being predicted to result in at least one peptide that is likely to be presented by an MHC molecule, the mutation being predicted to result in at least one peptide that is likely to be presented by an MHC allele that is known to be present in the subject, the mutation being likely to be clonal, and the mutation being predicted to result in a protein or peptide that is immunogenic. The step of selecting, by said processor, one or more of the tumour-specific mutations as candidate neoantigens, may comprise determining, by said processor, whether the one or more tumour specific mutations satisfy one or more predetermined criteria on whether the tumour-specific mutation is likely to be clonal selected from: the mutation having a likelihood of being clonal above a predetermined threshold, the mutation having a likelihood of being clonal that is above a threshold set adaptively to select a predetermined number of tumour-specific mutations with the highest likelihoods of being clonal amongst the tumour-specific mutations for which a likelihood was determined, and having a likelihood of being clonal that is above a threshold set adaptively to select a predetermined top percentile of tumour-specific mutations amongst the tumour-specific mutations for which a likelihood was determined.

The immunotherapy that targets the one or more of the selected neoantigens may be an immunogenic composition, a composition comprising immune cells or a therapeutic antibody. The immunotherapy may be a composition comprising T cells that recognise at least one of the one or more of the selected neoantigens identified. The composition may be enriched for T cells that target at least one of the one or more of the selected neoantigens identified. The method may comprise obtaining a population of T cells and expanding the population of T cells to increase the number or relative proportion of T cells that target at least one of the one or more of the selected neoantigens identified.

Determining a posterior probability that a candidate tumour-specific mutation is clonal depending on: a prior probability of the mutation being expressed, and the probabilities of observing the sequence data if the tumour-specific mutation is (i) expressed and (ii) not expressed, in view of a tumour fraction for each of the one or more samples and one or more candidate joint genotypes each comprising a genotype at the location of the tumour-specific mutation for a normal population, a reference tumour population that does not comprise the tumour-specific mutation and a variant tumour cell population that comprises the tumour-specific mutation) may be performed using the approach described in the following section.

### Identification of Clonal Mutations

Embodiments of the methods described herein may comprise determining whether a tumour-specific mutation is likely to be clonal. One possible method to determine this will now be described in more detail. The identification of candidate tumour-specific mutations that are likely clonal may use data comprising allele counts from N mutations (n=1, . . . , N) from S samples (s=1, . . . , S). For simplicity, and because the method can analyse a single sample and mutation, the indices n for the mutation and s for the sample will not be explicitly included in the notations used in this section. The model described in this section assumes that each mutation divides the set of cells that were sequenced into three sub-populations: (i) the normal cell population consisting of cells with healthy germline genomes (likely diploid in the region of the mutation); (ii) the reference cell population which consists of cancer cells without the mutation in question (may be aneuploid in the region of the mutation in question); and (iii) the variant cell population which consists of cancer cells with the mutation in question (may be aneuploid in the region of the mutation in question, may not have the same copy number in said region as the reference population). The term “mutation” is intended here in its broadest sense to refer to any genetic alteration that is detectable in sequence data, and particularly genomic sequence data. This includes in particular single nucleotide variants (SNVs), multiple nucleotide variants (MNVs), indels, etc.

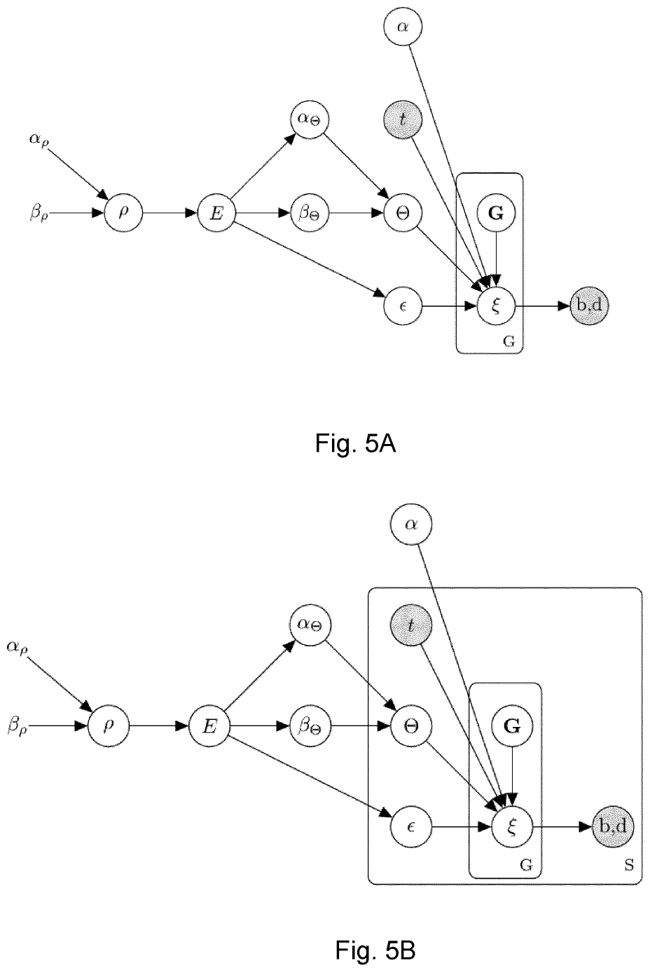

Let G=(A, B, AA, AB, AAA, AABB, . . . ) be the set of all genotypes where A and B represent reference and variant alleles respectively. For example, AB would represent a heterozygous variant (comprising one reference/normal allele A and one variant allele B) with total copy number 2. Under this notation, in FIG. 4, the normal population has the genotype AA (where both A can be the same or different, i.e. the normal population may be homozygous or heterozygous, but both alleles are normal), the reference population has the genotype AAA (where the A alleles are selected from the A alleles of the normal population), and the variant population has the genotype AABB (where the A alleles are selected from the A alleles of the normal population and the B alleles are any non-reference alleles). We assume that the genotype of all cells within each sub-population is constant (i.e. by reference to FIG. 4, all cells in the normal population have the genotype AA, all cells in the reference population have the genotype AAA, and all cells in the variant population have the genotype AABB). Let G=(G_{H};G_{R};G_{V})∈G³ be a vector where the entries are the genotype of the normal (healthy), reference and variant populations respectively (each of these individual genotypes will be referred to generically as “G” below). Let t be the proportion of cancer cells in the sample. This is often referred to as the tumour content, tumour purity or cellularity of the sample. Let $ be the proportion of cancer cells harbouring the mutation in the sample, that is the relative proportion of cancer cells in the variant population. This is often referred to as the cancer cell fraction (CCF) or cellular prevalence of the mutation. Let E be the assumed sequencing error rate. The following functions are defined:
a(G): G→ [symbol] is a function which maps a genotype to the number of A alleles (e.g., where G is AA, a(G)=2)
b(G): G→ [symbol] is a function which maps a genotype to the number of B alleles (e.g., where G is AA, b(G)=0)
c(G): G→ [symbol] is a function which maps a genotype to the total copy number at the locus (i.e. c(G)=a(G)+b(G); e.g. where G is AA, c(G)=2)
μ(G): G→ [symbol] is a function which maps a genotype to the value μ(G)=min{max{(b(G)/c(G)),ε}, (1−ε)}, which can be interpreted as the probability of sampling a read with the mutation from a population with genotype G.

Let ξ(G, φ,t) be the probability of sampling a read with the variant allele. Assuming that we have an infinite initial population of cells which are sampled when sequencing, the probability of sampling a read with a variant allele is roughly proportional to the number of copies of the variant allele in the input pool of DNA. More formally, accounting for sequencing error, the probability of sampling a variant allele (given a set of genotypes G, a tumour content t and a cancer cell fraction φ) is given by the following equation (equation (1)):

$$ξ ( G , ϕ , t ) = \frac{1}{T} ( 1 - t ) c ( {G}_{H} ) μ ( {G}_{H} ) + \frac{1}{T} t ( 1 - ϕ ) c ( {G}_{R} ) μ ( {G}_{R} ) + \frac{1}{T} t ϕ c ( {G}_{V} ) μ ( {G}_{V} ) \qquad ( 101 )$$

$$where T = ( 1 - t ) c ( {G}_{H} ) + t ( 1 - ϕ ) c ( {G}_{R} ) + t ϕ c ( {G}_{V} ) . \qquad ( 102 )$$

The variable ξ(G, φ,t) captures the sum of the number of copies of the variant allele originating from each genotype multiplied by the probability of sampling a read with a mutation from the genotype, normalised by the sum of the total number of copies of both alleles originating from each genotype.

The variable d is the total number of reads covering the mutation in the sample, of which d_{b} contain the mutant allele. Thus, the probability of observing these number of reads d, d_{b} (P(d, d_{b}|G, φ,t)) can be expressed with a Binomial model with parameters d_{b} and ξ(G, φ,t) (equation (3)). This is because the sum of m Bernouilli random variables with parameter p follow a Binomial distribution with parameters m, p². A Beta-binomial model with mean ξ(G, φ,t) and precision (inverse of variance) γ (equation (4)) can be used instead, for example if the data has more variance than can be explained by a Binomial model:

P(d,d_{b}|G,φ,t)=Binomial(d_{b}|d,ξ(G,φ,t)) (103)

P(d,d_{b}|G,φ,t,γ)=BetaBinomial(d_{b}|d,ξ(G,φ,t),γ) (104).

The parameters γ is set to 200 in the examples below, though other values are possible. So far, we have assumed that the genotypes of the sub-populations were known. In general this may be true for the healthy population (e.g. from a matched germline sample), but this is not true for the reference and the variant populations. Instead, it is typical to observe allele specific copy number estimates for the region overlapping a mutation. Using this information, we can elicit a prior over a set of plausible genotypes. We explain how to do this in the next section. For now assume we have a vector π of prior probabilities where πᵢ is the prior probability of the i^{th} plausible joint genotype, Gᵢ, of the populations. We can write the probability of the observed data marginalizing over all plausible genotypes as follows (equations (103a), (104a)):

P(d,d_{b}|π,φ,t)=Σᵢπᵢ Binomial(d_{b}|d,ξ(Gᵢ,φ,t)) (103a)

P(d,d_{b}|π,φ,t,γ)=ΣᵢπᵢBetaBinomial(d_{b}|d,ξ(Gᵢ,φ,t),γ) (104a).

In the subsequent sections, the notation Pr(d, d_{b}|π, φ,t) will be used to refer equally to the expression of equation (3a) and equation (4a). Note that φ and t are associated with individual samples so the notation above is a shorthand for φₛ and tₛ, respectively.

### Eliciting Mutational Genotype Priors

The above model uses either a known joint genotype, or prior probabilities π, where πᵢ is the prior probability of the i^{th} plausible joint genotype, Gᵢ, of the populations (i.e. Gᵢ is one possible combination of genotypes for the healthy, variant and reference populations). Various methods can be used to set potential genotype priors. Note that the same principles apply to the methods for determining whether a tumour-specific mutation is likely to be expressed, with the difference that the joint genotypes G refer to the genotypes of the tumour (variant) and normal (no variant/germline) population (i.e. there is no reference tumour population).

For example, one possible method can be referred to as the “major copy number” method. Let cₘₐⱼₒᵣ and cₘᵢₙₒᵣ denote the major and minor allele copy number for the region overlapping the mutation in the tumour sample. The method “major copy number method” considers two cases:
(a) In the first case, the mutation occurs before the copy number event. In this case the reference population genotype matches the normal population. We consider all possible mutational genotypes for the variant population with up to cₘₐⱼₒᵣ chromosomes containing the variant.
(b) In the second case, the mutation occurs after the copy number event. In this case the reference population has cₘₐⱼₒᵣ+cₘᵢₙₒᵣ reference alleles. The variant population has 1 variant allele and cₘₐⱼₒᵣ+cₘᵢₙₒᵣ−1 reference allele.

We set the prior weights to be equal for all possible mutational genotypes. For example suppose we have that cₘₐⱼₒᵣ=2 and cₘᵢₙₒᵣ=1 and the normal copy number is 2. We have the following possible genotypes:
G₁=(AA, AA, AAB)
G₂=(AA, AA, ABB)
G₃=(AA, AAA, AAB)
   each with a prior probability of ⅓. Note that if allele specific copy number is not available then cₘₐⱼₒᵣ can be set to the total copy number and cₘᵢₙₒᵣ to zero. This approach assumes that a mutation occurs only once, such that if more than one copy of the mutant allele is present in the variant population, then this occurred because the mutation preceded a copy number change at the locus and was subsequently amplified. This approach strikes a good balance between accounting for uncertainty in the genotypes of the populations while not considering too many states.

In the context of determining whether a tumour-specific variant is expressed, the following possible genotypes are considered in the example above: G₁=(AA, AAB), G₂=(AA, ABB), G₃=(AA, AAB). References below to using the maximum a posteriori (MAP) estimates of genotypes refer to the use of the MAP estimates of the major and minor copy number and purity from DNA sequence data for the sample(s), using the above major copy number method.

Alternative approaches may be used for setting the mutational genotype priors. Another possible approach is to simply assume that each mutation is diploid and heterozygous (i.e. the variant in the variant population only occurs on one of the two chromosomes, G=(G_{H}=AA, G_{R}=AA, G_{V}=AB), or G=(G_{H}=AA, G_{V}=AB) in the context of determining whether a tumour-specific mutation is expressed). This may be referred to as “AB prior”. Yet another simplistic approach is to assume that each mutation is diploid and homozygous (i.e. the variant in the variant population occurs on both of the two chromosomes, G=(G_{H}=AA, G_{R}=AA, G_{V}=BB), or G=(G_{H}=AA, G_{V}=BB) in the context of determining whether a tumour-specific mutation is expressed). This may be referred to as “BB prior”. Yet another possible simple approach is to assume that the genotype of the variant population has the predicted total copy number at the region of the mutation, with exactly one mutant allele (i.e. assuming that the total copy number is 3,G=(G_{H}=AA, G_{R}=AA, G_{V}=AAB), or G=(G_{H}=AA, G_{V}=AAB) in the context of determining whether a tumour-specific mutation is expressed, i.e. this results in considering only G₁ in the “major copy number” method above). This may be referred to as “no zygosity prior”. These approaches may be too simplistic in many cases as they essentially consider a single possible genotype.

Another possible approach is to assume that the genotype of the variant population has the predicted total copy number at the region of the mutation, with at least one mutant allele, and that the reference population is either AA or the genotype with a copy number equal to the predicted total copy number and no variant allele (with equal probability). This may be referred to as the “total copy number prior” and intuitively means that the genotype of the variant population at the locus has the predicted total copy number and may have any number (>0) of copies of the mutant allele (i.e. assuming that the total copy number is 3, the possible genotypes are, with equal probabilities, G₁=(G_{H}=AA, G_{R}=AA, G_{V}=AAB), G₂=(G_{H}=AA, G_{R}=AA, G_{V}=ABB), G₃=(G_{H}=AA, G_{R}=AA, G_{V}=BBB), G₄=(G_{H}=AA, G_{R}=AAA, G_{V}=AAB), or the same genotypes without G_{R} in the context of determining whether a tumour-specific variant is expressed, i.e. this essentially ignores the major and minor copy number values and considers all possible genotypes with n copies—leading to an additional genotype being considered compared to the “major copy number” method above). Yet another approach that can be used is to “trust” the predicted number of major and minor alleles from the copy number caller, such that only genotypes that have a number of mutant alleles corresponding to either the major copy number or the minor copy number are considered. This may be referred to as the “parental” mode. For example, if major copy number=3, minor copy number=1, then this approach would consider the following possible genotypes, with equal probabilities: G₁=(AA, AA, AAAB), G₂=(AA, AA, ABBB), G₃=(AA, AAAA, AAAB) (i.e. either 1 or 3 mutated alleles in the variant population), or the same genotypes without G_{R} in the context of determining whether a tumour-specific variant is expressed. By contrast, the “major copy number” approach “trusts” the range of the possible major copies, but not the absolute value of it, by considering all values between 1 and the predicted major copy number. With the example above of major copy number=3, minor copy number=1, this would lead to one more genotype being considered compared to the “parental” mode, i.e.: G₁=(AA, AA, AAAB), G₂=(AA, AA, AABB), G₃=(AA, AA, ABBB), G₄=(AA, AAAA, AAAB), or the same genotypes without G_{R} in the context of determining whether a tumour-specific variant is expressed. Thus, the “major copy number” approach strikes a good balance between accounting for additional uncertainty from the copy number calls (compared to the “parental” approach) without having consider too much uncertainty (compared to the “total copy number” approach).

### Clonality Estimation Model

A hierarchical Bayesian model may be used based on the above for identifying ubiquitous mutations. Let Z be a Bernoulli variable which is one when a mutation is ubiquitous (assumed to be clonal) and zero otherwise. Let ρ be the prior probability that the mutation is ubiquitous. This is set to 0.5 in the examples below. As above, φ is the proportion of cancer cells harbouring the mutation in the sample. Thus, the model can be expressed as:

Z|ρ~Bernoulli(Z|p) (105)

φ|Z~Beta(φ|αa=1,β=1) for Z=0;

Beta(φ|α,β=1) for Z=1 (106)

d_{b},d|π,φ,t~Pr(d,d_{b}|π,φ,t) (107)

where α is a parameter >1 in the distribution of φ|Z=1. This is set to α=99 in the examples below. A Beta distribution with parameters α=99 and β=1 is skewed towards 1, capturing the assumption that clonal mutations should be enriched for higher cancer cell fraction φ. Other values of the parameter α are possible, though values that capture this assumption are preferred. As mentioned above, the probability in equation (107) is given by equations (103)/(103a) or (104)/(104a).

The joint distribution can be expressed with the following equation (equation (108)):

p(d_{b},d,φ,Z=z|π,t,ρ)=p(Z=z|ρ)Pr(d_{b},d|π,φ,t)p(φ|Z=z) (108)

for one sample, or for a plurality of samples:

$$p ( {d}_{b} , d , ϕ , Z = z \vert π , t , ρ ) = p ( Z = z \vert ρ ) {\prod }_{s = 1}^{S} Pr ( {d}_{b} , d \vert π , ϕ , t ) p ( ϕ \vert Z = z ) \qquad ( 108 a )$$

The proportion of cancer cells harbouring the mutation (φ) is unknown. However, we can express:

$$p ( {d}_{b} , d , Z = z \vert π , t , ρ ) = {\int }_{0}^{1} P ( {d}_{b} , d , ϕ , Z = z \vert π , t , ρ ) d ϕ = P ( Z = z \vert ρ ) {\int }_{0}^{1} P r ( {d}_{b} , d \vert π , ϕ , t ) p ( ϕ \vert Z = z ) d ϕ \qquad ( 109 )$$

for one sample, or for multiple samples:

$$p ( {d}_{b} , d , Z = z \vert π , t , ρ ) = P ( Z = z \vert ρ ) {\prod }_{s = 1}^{S} {\int }_{0}^{1} Pr ( {d}_{b} , d \vert π , ϕ , t ) p ( ϕ \vert Z = z ) d ϕ . \qquad ( 109 a )$$

The quantity

$${\prod }_{s = 1}^{S} {\int }_{0}^{1} Pr ( {d}_{b} , d \vert π , ϕ , t ) p ( ϕ \vert Z = z ) d ϕ$$

may be referred to as ψ_{z} (i.e. ψ₀ and ψ₁ respectively referring to the likelihood of the data if the mutation is non clonal and if the mutation is clonal). As P(Z=z|ρ)=(1−ρ) for z=0 (i.e. the prior probability of Z=0, i.e. the mutation being classified as non-clonal, given a prior probability ρ of the mutation being clonal is equal to the prior probability of the mutation not being clonal), and P(Z=z|ρ)=ρ for z=1 (i.e. the prior probability of Z=1, i.e. the mutation being classified as clonal, given a prior probability of the mutation being clonal of ρ is equal to the prior probability of the mutation being clonal), it follows that:

$$p ( {d}_{b} , d \vert π , t , ρ ) = {\sum }_{z = 0}^{z = 1} p ( {d}_{b} , d , Z = z \vert π , t , ρ ) = ( 1 - ρ ) {\prod }_{s = 1}^{S} {\int }_{0}^{1} Pr ( {d}_{b} , d \vert π , ϕ , t ) p ( ϕ \vert Z = 0 ) d ϕ + ρ {\prod }_{s = 1}^{S} {\int }_{0}^{1} Pr ( {d}_{b} , d \vert π , ϕ , t ) p ( ϕ \vert Z = 1 ) d ϕ \qquad ( 110 )$$

for multiple samples (without the product over samples for a single sample).

Ultimately, the quantity that we wish to estimate is the probability of a mutation being clonal (probability that Z=1), in view of the reads observed (d_{b}, d), a genotype prior (π), a tumour fraction estimate (t), and a prior probability of the mutation being clonal (p, i.e. we want to estimate P(Z=1|d_{b}, d, π,t, p)). In view of the above, this can be expressed as:

$$p ( Z = z \vert {d}_{b} , d , π , t , ρ ) = \frac{p ( {d}_{b} , d , Z = z \vert π , t , ρ )}{p ( {d}_{b} , d \vert π , t , ρ )} \qquad ( 111 )$$

where p(d_{b}, d|π,t, ρ) is given by equation (110) and p(d_{b}, dZ=z|π,t, p) is given by equations (109)/(109a). Thus, equation (111) can be written for Z=1 as equation (111a) below:

$$p ( Z = 1 \vert {d}_{b} , d , π , t , ρ ) = \frac{ρ {\prod }_{s = 1}^{S} {\int }_{0}^{1} Pr ( {d}_{b} , d \vert π , ϕ , t ) p ( ϕ \vert Z = 1 ) d ϕ}{\begin{matrix}( 1 - ρ ) {\prod }_{s = 1}^{S} {\int }_{0}^{1} Pr ( {d}_{b} , d \vert π , ϕ , t ) p ( ϕ \vert Z = 0 ) d ϕ + \\ ρ {\prod }_{s = 1}^{S} {\int }_{0}^{1} Pr ( {d}_{b} , d \vert π , ϕ , t ) p ( ϕ \vert Z = 1 ) d ϕ\end{matrix}} \qquad ( 111 a )$$

where ρ is a parameter (set to 0.5 in the examples below), p(π|Z=z) is given by the beta distributions in equation (106), and Pr(d_{b}, d|π, φ,t) is given by equations (103)/(104) (one joint genotype) or (103a)/(104a) (plurality of candidate joint genotypes with prior probabilities π).

Thus, estimating equation (111) for z=1 (i.e. equation (111a)) gives us the probability that a mutation is ubiquitous (i.e. assumed to be clonal in view of the one or more samples available). This requires evaluating S one dimensional integrals (one for each sample, in equations (109), (110)), which can be done efficiently using known numerical integration. Any numerical integration algorithm known in the art may be used for this purpose. For example, a grid approximation may be used. This is advantageously simple, and sufficient considering that there is a single parameter (φ) to integrate over.

This provides an estimate of the probability that a mutation is clonal in view of the data available, which can be efficiently computed, is readily interpretable (in view of the rigorous statistical model making use of explicit clear assumptions), can be obtained for any mutation without manual input, is independent of any other mutation analysed, can rigorously include prior knowledge about the mutation, and can be used to objectively and automatically prioritise a list of mutations (with accompanying probabilities) for testing and/or use.

### Accounting for Uncertainty in Copy Number Predictions

While the model described above already presents numerous advantages, it can be further enhanced by taking into account uncertainties in the prediction of the copy number estimates used in the model. Indeed, the above model assumes that the copy numbers (e.g. the major/minor/total/copy numbers used to elicit the genotype priors) were accurately predicted. In practice there may be some uncertainty in these values. Indeed, the problem of allele-specific copy number analysis of tumours is complex and many solutions have been proposed to do this. One commonly used approach is ASCAT (allele-specific copy number analysis of tumors, Van Loo et al., 2010), which takes into account both aneuploidy of the tumour cells and non-aberrant cell infiltration in interpreting a bulk copy number profile, and outputs estimated allele-specific copy number profiles and accompanying tumour purity estimates. In short, ASCAT evaluates a plurality of possible combinations of tumour ploidy and tumour fractions, based on the assumption that the associated allele-specific copy number calls should be as close as possible to nonnegative whole numbers for germline heterozygous single nucleotide polymorphisms (SNPs). A solution deemed optimal is then reported (estimated tumour ploidy, tumour purity and allele-specific copy number calls for the tumour and normal part of the sample) together with its goodness-of-fit (based on the above assumption).

The model provided above can be adjusted to accommodate multiple copy number solutions and their uncertainties, by modifying π to contain entries for the genotypes from each predicted copy number state (e.g. each proposed solution comprising a major and minor copy state), weighted by the probability associated with this state. Additionally, as the tumour purity estimate may be estimated together with these copy number states (as is the case e.g. when an approach like ASCAT is used), the associated tumour purity estimate can also be taken into account. Note that this may not be necessary when e.g. the tumour purity is estimated or measured separately and is not intrinsically associated with the copy number state estimate. Nevertheless, for the sake of generality, let us assume that we have a set of C possible copy number/tumour content states (e.g. C possible sets of estimates of cₘₐⱼₒᵣ, cₘᵢₙₒᵣ, and t). Let π_{C} be a vector where each entry is the probability for each possible such set of estimates. For each state C, it is possible to compute the vector π_{CG} of possible genotypes as explained above. A final genotype vector can thus be obtained by multiplying π_{CG} by the entry for state C in π_{C}. This gives rise to the slightly modified equations below:

P(d,d_{b}|π,φ)=Σᵢπᵢ Binomial(d_{b}|d,ξ(Gᵢ,φ,tᵢ)) (103b)

P(d,d_{b}|π,φ,γ)=Σᵢπᵢ BetaBinomial(d_{b}|d,ξ(Gᵢ,φ,tᵢ),γ) (104b).

where the tumour content tᵢ may now depend on the particular state (and the πᵢ are elements of the vector π obtained by multiplying π_{CG} by the entry for state C in π_{C}). These new densities can be substituted in the relevant equations above. In particular, the problem solved may then be expressed as solving equation (111a), where Pr(d_{b}, d|π,φ,t) is given by equation (103b) or equation (104b). The values for tᵢ, cₘₐⱼₒᵣ, cₘᵢₙₒᵣ (and hence the compatible π_{CG} according to the model used) and π_{C} are provided as outputs of many methods for performing allele-specific copy number analysis of tumours, including but not limited to ASCAT, as explained above. For the avoidance of any doubt, any approach that generates allele-specific copy number state estimates (typically with associated with a tumour purity estimate) with a confidence or other metric that can be used to weight multiple solutions relative to each other may be used for this purpose.

### Implementation

The methods described throughout this specification may be implemented using any programming language known in the art. For example, for the purpose of identifying clonal mutations, a Python script implementing the above method may be used, taking as input for each mutation: a mutation identifier, a sample identifier, a count of the number of reads that match the reference allele at the mutation position, a count of the number of reads that match the alternate allele at the mutation position, and, for each of one or more copy number solutions: the major copy number (for the tumour) overlapping the mutation for the specified copy number solution, the minor copy number (for the tumour) overlapping the mutation for the specified copy number solution, a copy number for the normal cell at the mutation (may be set to default=2 for autosomal chromosomes, or 1 for a sex chromosome in a male subject), and a tumour purity value for the specified copy number solution (this can also be obtained as an output of e.g. ASCAT, or can be separately obtained). The major and minor copy number overlapping the mutation for the tumour population, for a specified copy number solution, can be obtained directly from ASCAT (e.g. using ascatNgs, Raine et al., 2016), or derived from the output of e.g. ASCAT such as using the mean B allele frequency of the copy number segment overlapping the mutation, the log R value of the copy number segment overlapping the mutation, and the ploidy of the solution. For example, the allele specific copy number estimates ( [symbol] , [symbol] ) for the tumour at a location i can be expressed as functions of the log R value rat location i, the B allele fraction value b at location i, the ploidy estimate ψ, the tumour cell fraction estimate ρ, and a platform-dependent “technology” parameter t (which can be set to t=1 for next generation sequencing data such as WES) using:

$$\text{?} = ( ρ - 1 + {2}^{\frac{r}{ϒ}} ( 1 - b ) ( 2 ( 1 - ρ ) + ρ ψ ) ) / ρ and$$

$$\text{?} = ( ρ - 1 + {2}^{\frac{τ}{ϒ}} b ( 2 ( 1 - ρ ) + ρ ψ ) ) / ρ .$$

The major and minor copy numbers in the normal population may be assumed to be 1 and 1, apart from mutations on the sex chromosomes which may be handled depending on the sex of the subject. Where multiple copy number solutions are provided, a probability of each solution may optionally be provided (this can also be obtained from the output of e.g. ASCAT which proves a negative log likelihood for a solution). If this is not provided, then all of a plurality of solutions may be treated as equally likely and receive equal weight. The script may produce as output a mutation identifier and posterior probability that the mutation is clonal/ubiquitous.

As another example, for the purpose of identifying expressed mutations, a Python script implementing the above method may be used, taking as input for each mutation: a mutation identifier, a sample identifier, a count of the number of RNA reads that match the reference allele at the mutation position, a count of the number of RNA reads that match the mutant allele at the mutation position, and, a genotype for the normal and tumour cells (e.g. obtained using ASCAT and/or any approach described herein for inferring joint genotypes for tumour and normal populations from mixed samples), and a tumour purity value (optionally associated with the specific joint genotype, such as e.g. obtained as an output of e.g. ASCAT). The script may produce as output a mutation identifier and posterior probability that the mutation is expressed.

### Systems

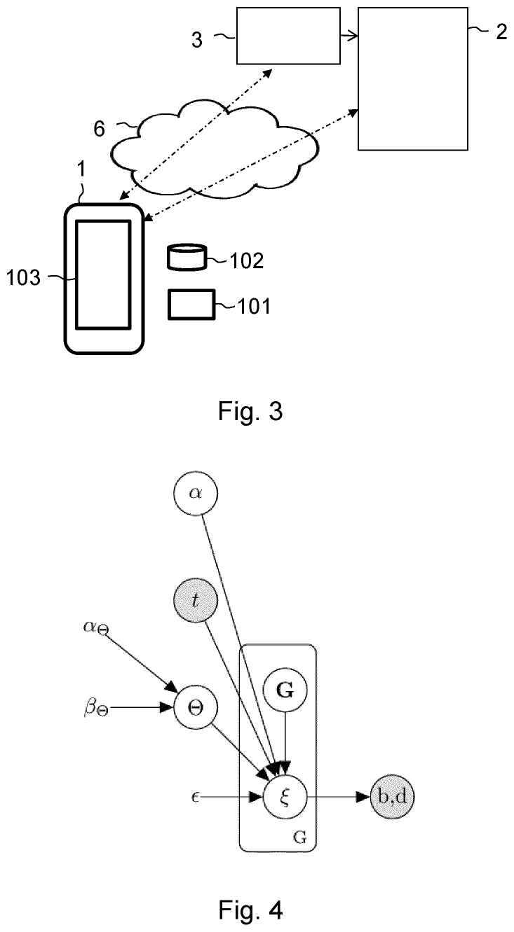

FIG. 3 shows an embodiment of a system for determining whether a tumour-specific mutation is likely to be expressed, and/or identifying neoantigens and/or for providing an immunotherapy based at least in part on the identified neoantigens, according to the present disclosure. The system comprises a computing device 1, which comprises a processor 101 and computer readable memory 102. In the embodiment shown, the computing device 1 also comprises a user interface 103, which is illustrated as a screen but may include any other means of conveying information to a user such as e.g. through audible or visual signals. The computing device 1 is communicably connected, such as e.g. through a network 6, to sequence data acquisition means 3, such as a sequencing machine, and/or to one or more databases 2 storing sequence data. The one or more databases may additionally store other types of information that may be used by the computing device 1, such as e.g. reference sequences, parameters, etc. The computing device may be a smartphone, tablet, personal computer or other computing device. The computing device is configured to implement a method for determining whether a tumour specific mutation is likely to be expressed, as described herein. In alternative embodiments, the computing device 1 is configured to communicate with a remote computing device (not shown), which is itself configured to implement a method of determining whether a tumour specific mutation is likely to be expressed, as described herein. In such cases, the remote computing device may also be configured to send the result of the method to the computing device. Communication between the computing device 1 and the remote computing device may be through a wired or wireless connection, and may occur over a local or public network such as e.g. over the public internet or over WiFi.

The sequence data acquisition 3 means may be in wired connection with the computing device 1, or may be able to communicate through a wireless connection, such as e.g. through a network 6, as illustrated. The connection between the computing device 1 and the sequence data acquisition means 3 may be direct or indirect (such as e.g. through a remote computer).

The sequence data acquisition means 3 are configured to acquire sequence data from nucleic acid samples, for example RNA samples and also optionally genomic DNA samples extracted from cells and/or tissue samples. In some embodiments, the sample may have been subject to one or more preprocessing steps such as RNA purification, fragmentation, library preparation, target sequence capture (such as e.g. exon capture and/or panel sequence capture). Preferably, the sample has not been subject to amplification, or when it has been subject to amplification this was done in the presence of amplification bias controlling means such as e.g. using unique molecular identifiers. Any sample preparation process that is suitable for use in the determination of a genomic copy number profile (whether whole genome or sequence specific) may be used within the context of the present disclosure. The sequence data acquisition means is preferably a next generation sequencer. The sequence data acquisition means 3 may be in direct or indirect connection with one or more databases 2, on which sequence data (raw or partially processed) may be stored.

The following is presented by way of example and is not to be construed as a limitation to the scope of the claims.

### EXAMPLES

These examples describe a method of identifying clonal mutations according to the present disclosure, and demonstrate its use using simulated data and multiple types of experimental data.

### Introduction

Allele expression (AE, also referred to herein as ‘allele-specific expression’, ASE) has been reported as a predictor of immunogenicity (Gartner et al. 2021). While it may be possible to determine if the mutant allele is expressed by checking for the presence of variant reads in RNA-seq data, the power to detect its expression depends on factors such as tumour purity/gene expression level, and we cannot distinguish between lack of ASE and power to detect ASE.

Additionally, immunogenic mutations without ASE could potentially be due to immuno-editing (the tumour cells have repressed the expression to avoid immune detection). This is important because if we had a power to detect measure we can separate out those that are 1) potentially false negative (low power to detect), 2) potentially immuno-edited (high power to detect).

The most basic approach is to check if >=1 variant read(s) in the sequencing reads cover a mutation loci and determined that: (i) there is no AE if 0 variant reads are observed; (ii) there is AE if >=1 variant read are observed. However, the chance of observing >=1 variant read when the mutation was truly expressed depends on the sample tumour purity, genotype, and gene expression level. At 100% purity and high gene expression, there is a high chance of seeing >=1 variant read(s) if the mutation was truly expressed. At 5% purity and low gene expression, there is a low chance of seeing >=1 variant read(s) if the mutation was truly expressed.

In this work, the inventors present a series of statistical methods called ALExA (Achilles Likelihood of an Expressed Allele) for evaluation of allele specific expression in a tumour context. In particular, the inventors devised a method that calculates the power of detecting a mutation as being expressed, accounting for tumour purity, genotype and gene expression (Example 1). The inventors further present a statistical method that estimates a probability of a mutation being expressed given RNAseq read count data (Example 2), using the concepts developed in Example 1.

### Example 1—Power to Detect Allele-Specific Expression in a Tumour

This section presents a model for the number of variant reads that are expected to be observed if a variant is expressed or is not expressed (illustrated on FIG. 4). This is used to derive a likelihood of observing a given set of data if the variant is expressed or is not expressed, which in turn is framed in a statistical hypothesis context to identify the power to detect a variant that is expressed at a chosen false positive rate.

### Methods

### Modelling the Number of Variant Reads

The model assumes that each mutation divides the set of cells that were sequenced into two sub-populations:
the normal cell population consisting of cells with healthy germline genomes, with genotype G_{N};
the variant cell population which consists of cancer cells with the mutation in question, with genotype G_{V}

The model assumes that the mutation is clonal (in the samples under investigation), and the cancer cell fraction φ in the tumour is 1 (i.e. all the cancer cells harbour the variant). It would be possible to extend to subclonal mutations by placing a prior on φ (for instance a Beta distribution) and including a reference tumour population in the genotypes as explained above in relation to the determination of whether a mutation is clonal. The prior on φ may be for example obtained from analysis of DNA sequence data as explained above. Note that the variant cell population which consists of cancer cells with the mutation in question may be aneuploid in the region of the mutation in question, may not have the same copy number in said region as the normal population. The term “mutation” is intended here in its broadest sense to refer to any genetic alteration that is detectable in sequence data, and particularly genomic sequence data. This includes in particular single nucleotide variants (SNVs), multiple nucleotide variants (MNVs), indels, etc. The present methods relate to the detection of variants present in a tumour population and therefore the mutations are somatic mutations.

Let G=(A, B, AA, AB, AAA, AABB, . . . ) be the set of all genotypes where A and B represent reference and variant alleles respectively. For example, AB would represent a heterozygous variant (comprising one reference/normal allele A and one variant allele B) with total copy number 2. Thus, the normal population has the genotype AA (where both A can be the same or different, i.e. the normal population may be homozygous or heterozygous, but both alleles are normal), and a tumour population may for example have the genotype AABB (where the A alleles are selected from the A alleles of the normal population and the B alleles are any non-reference alleles). The following notation is used:
d is the total number of reads covering the mutation in the sample;
b is the number of reads containing the mutation in the sample;
ε is the assumed sequencing error rate;
a(G): G→ [symbol] is a function which maps a genotype to the number of A (reference) alleles (e.g., where G is AA, a(G)=2);
b(G): G→ [symbol] is a function which maps a genotype to the number of B (variant) alleles (e.g., where G is AA, b(G)=0);
c(G): G→ [symbol] is a function which maps a genotype to the total copy number at the locus (i.e. c(G)=a(G)+b(G); e.g. where G is AA, c(G)=2);
t is the tumour purity (the proportion of cancer cells in the sample); the value of this parameter can be assumed, known or estimated from e.g. DNA sequence data associated with the sample(s) under investigation;
G={G_{N};G_{V}}∈G² is a vector where the entries are the genotype of the normal (healthy), and variant populations respectively; the value of this parameter can be assumed, known or estimated from e.g. DNA sequence data associated with the sample(s) under investigation;
Θ is the reference ratio (reference read count/total read count), the fraction of total read counts attributed to the reference allele; this parameter gets integrated over and as such its exact value is not determined;
α is the fraction of total expression of the gene comprising the tumour-specific mutation which is due to the normal cell population (e.g. TPMₙₒᵣₘₐₗ/(TPMₙₒᵣₘₐₗ+TPMₜᵤₘₒᵤᵣ), where TPM are transcripts per million, a common expression metric in RNA sequencing); the value of this parameter can be assumed, known or estimated from e.g. RNA sequence data from a plurality of samples with different purities as will be explained further below;
μ(G): G→ [symbol] is the probability of sampling a read with the mutation from a population with genotype G, defined as μ(G,Θ, ∈)=p(m=1|G,Θ), i.e. the probability that a read picked from a sequenced cell population with genotype G will carry the mutation of interest (m=1).
   Probability of Sampling a Read with the Mutation from a Population with Genotype G

The model presented herein models the process of sampling RNA-seq reads with a mutation of interest from a tumour sample (where a tumour sample is assumed to comprise a mixed population that may include cells with at least some copies of the mutation—i.e. tumour cells—and cells without any copies of the mutation—i.e. normal cells), allowing for different allelic expression of mutant and reference alleles. The probability of sampling a read with the mutation of interest from a population sequenced with genotype G is obtained as p(m=1|G, Θ) (i.e. the probability that a read picked from a sequenced cell population with genotype G will carry the mutation of interest (m=1)) provided by equation (1):

$$μ ( G , Θ , ε ) = p ( m = 1 \vert G , Θ ) = \{ \begin{matrix}\frac{b ( G ) ( 1 - θ )}{b ( G ) ( 1 - θ ) + α ( G ) θ} if b ( G ) > 0 , b ( G ) \neq c ( G ) \\ ε if b ( G ) = 0 \\ 1 - ε if b ( G ) = c ( G )\end{matrix} \qquad ( 1 )$$

where the first case captures a variant population that contains at least one reference allele, where it is expected that b(G_{V})>0, the second case captures the normal population, and the third case captures a variant population that does not contain any reference alleles. In other words, the probability of sampling a read with the variant allele from a population that has at least one variant allele and at least one reference allele is equal to

$$\frac{b ( G ) ( 1 - θ )}{b ( G ) ( 1 - θ ) + α ( G ) θ} ,$$

the probability of sampling a read with the variant allele from a population that has no variant allele is equal to ε, and the probability of sampling a read with the variant allele from a population that has no reference allele is equal to 1−ε.

Note that this model assumes that the sequencing errors are independent of the allele. If this is not the case, separate error rates may be used depending on the type of mutation. For example, for substitutions, different error rates may be provided for classes of substitutions (e.g. transitions/transversions or individual mutations e.g. A->C, A->G, A->T). Additionally, there may be instances where aligning and calling the variant allele may be more prone to error, compared to the reference allele. For example, reads aligning to variant alleles may be undercounted in some situations such as for some indel variants. This can be addressed by adding a bias term in the model to correct the observed number of variant reads, for example depending on the expected proportion of reads that may fail to align to the variant.

Probability of Sampling a Read with the Mutation from a Tumour Sample (Mixed Population)

Assuming an infinite initial population of cells which are sampled when sequencing, such that the probability of sampling a read with the variant allele is proportional to the number of copies of the variant allele (b(G) for each genotype) and its relative expression (compared to the reference allele,θ), the probability of sampling a read with the mutation from the tumour sample (given a set of genotypes G, a tumour content t and a normal expression fraction a) is provided by equations (2)-(2″):

ξ(G,θ,α,t)=p(m=1|G,θ,α,t) (2)

ξ(G,θ,α,t)=Σ_{P∈{V,N}}p(m=1|G_{P},θ,ε)p(P|t) (2′)

$$ξ ( G , θ , α , t ) = \frac{α ( 1 - t ) c ( {G}_{N} )}{α ( 1 - t ) c ( {G}_{N} ) + ( 1 - α ) t c ( {G}_{V} )} μ ( {G}_{N} , θ , ε ) + \frac{( 1 - α ) t c ( {G}_{V} )}{α ( 1 - t ) c ( {G}_{N} ) + ( 1 - α ) t c ( {G}_{V} )} μ ( {G}_{V} , θ , ε ) \qquad ( {2}^{″} )$$

### Model Likelihood

When we observe d total reads covering the mutation in the sample, of which b contain the mutant allele, the likelihood of observing these number of reads is given by equation (3):

P(b,d|G,θ,α,t)=Binomial(b|d,ξ(G,θ,α,t)) (3)

Let E be a binary variable representing whether the mutant allele at a site is expressed (E=1). The following priors on the reference ratio Θ are used:

p(θ|E=1)=Beta(θ,α₁,β₁) (4)

p(θ|E=0)=Beta(θ,α₀,β₀)

where the indices “0” refer to the null model (E=0) denoted M₀ (the mutant allele is not expressed), and the indices “1” refer to the alternative model (E=1) denoted M₁ (the mutant allele is truly expressed). The parameters of these models are chosen to give a broad prior for E=1 and a peaked probability mass around 1 when E=0. In particular, in these examples, α₀=9999, β₀=1, α₁=1, and β₁=1.

For the M₀ and M1 models, we have:

$$P ( b \vert {M}_{0} ) = {\sum }_{g = 1}^{G} \int P ( b \vert d , {G}_{g} , θ , α , t ) p ( θ \vert E = 0 ) p ( {G}_{g} ) d θ \qquad ( 5 )$$

$$P ( b \vert {M}_{1} ) = {\sum }_{g = 1}^{G} \int P ( b \vert d , {G}_{g} , θ , α , t ) p ( θ \vert E = 1 ) p ( {G}_{g} ) d θ$$

where we integrate over all the possible genotypes, supposing that there are G possible genotype sets, where the gth genotype set Gg={G_{N},G_{V}}_{g} occurs with probability p(G_{g}). The value of P(b|d, G_{g},θ,α,t) is provided by equation (3), and the value of p(θ|E=0) and p(θ|E=1) is provided by equation (4). In the present examples, the genotypes G were estimated by obtaining the major and minor copy number from DNA sequence data from the samples, and using the major copy number approach described above to obtain genotype estimates. In particular, this approach may consider that the normal population has a genotype AA and that the variant population has any of the genotypes with the number of variant alleles (B) from 1 to the major copy number. If there has been a copy number gain or loss (the major and minor copy numbers do not sum to 2), the approach accounts for two cases: a case where the mutation occurred before the copy number event (e.g. if minor copy number=1, major copy number=2: G_{V}=AAB, ABB), and a case where the mutation occurred after the copy number event (G_{V}=AAB), resulting in a genotype distribution for the variant population of G_{V}=AAB with probability ⅔ and G_{V}=ABB with probability ⅓.

The sequencing error rate was additional set differently for the null model (M0) and the alternative model (M1). For the null model, the sequencing error rate was set to a value ε0=0.001. This assumes that variant reads may be present with a very small probability simply due to sequencing errors. For the alternative model, the sequencing error rate was set to ε0=0.

This assumes that all variant reads are due to true variants rather than sequencing errors. A single value may alternatively be used for both models.

Note that both b and d are observed variables, but the approach only models b as d is treated as a parameter of the model. Thus, terms such as P(b|M₀) are equivalent to P(b,d|M₀) (and similarly for P(b|M₁)=P(b,d|M₁), P(b|d, G_{g},θ,α,t)=P(b,d|G_{g},θ,α,t), P(b,d|α,t,E=1)=P(b|d,α,t,E=1), etc.)

Edge Case for Variant Population with No Reference Allele

The model was further designed to allow for the edge case of a genotype G_{V} where a(G_{V})=0, i.e. there are no reference alleles. This is because for this genotype, (with εᵢ the sequencing noise parameter for model Mᵢ):

$$μ ( {G}_{V} , Θ , ε ) = p ( m = 1 \vert {G}_{V} , Θ ) = \{ \begin{matrix}1 - {ε}_{0} & M = {M}_{0} \\ 1 - {ε}_{1} & M = {M}_{1}\end{matrix} \qquad ( 6 )$$

i.e. the probability of sampling a read with the mutation from the variant population does not depend on θ and the null and alternative models are essentially the same, ξ(G,θ,α,t)~t for both models, assuming εᵢ~0, α=0.5 and c(G_{V})=c(G_{N}). Thus, for this genotype there would be a low power to detect VSE (as both models are the same), which does not seem intuitive as if the variant genotype only contains the variant allele, and t is high, then one would expect a high power to detect variant reads. Intuitively, if the variant population contains only variant alleles, under the null hypothesis of no variant expression, one would expect to see lower coverage than under the alternative hypothesis of variant expression. Thus, the model was adapted to set α as:

$$α = \{ \begin{matrix}1 & for M = {M}_{0} α ( {G}_{V} ) = 0 \\ {α}_{ξ} & otherwise\end{matrix} \qquad ( 7 )$$

Where α_{ε} is the value of α for all cases other than the case where the variant population contains no reference allele. This can be set to a default value, such as e.g. 0.5, or it can be estimated from data as explained further below. As a result, this sets ξ(G,θ,t)=ε(equation (2), which feeds into equation (3) which itself feeds into equation (5)) for the null model M₀ in the particular case where the variant genotype G_{V} has no reference allele.

### Power to Detect an Expressed Mutation

When assessing RNAseq data to decide whether a mutant allele is expressed in a sample, there are four different scenarios:
true positive: the mutated allele is expressed and expression is detected;
false positive: the mutated allele is not expressed and expression is detected (Type I error)
true negative: the mutated allele is not expressed and expression is not detected
false negative: the mutated allele is expressed and expression is not detected (Type II error).

We want to test at each mutated site and decide whether the mutated allele is expressed or not. Given the null model M₀, we can calculate the rejection region for a hypothesis test of e.g.:

H₀: α₀=999,β₀=1

H₁: α₀≠999,β₀≠1

i.e. we can find the critical value b_{c} (critical number of reads containing the mutation) such that we reject H0 when b≥b_{c}. The critical value b, is obtained by calculating the distribution of P(b|M₀) over the number of variant reads using equation (5), and obtaining the cumulative distribution to determine the value of b where P(b≥b_{c})<0.05. We can use the test statistic b with:
reject H₀ if b≥b_{c} with significance level P(b≥b_{c}|M₀)
accept H₀ if b<b_{c}

Assuming now that the variant allele is expressed, and data really comes from the alternative model with α₁=1, and β₁=1. Then:
if b<b_{c} we accept the null hypothesis. However, given that H₁ is in fact true, this is a false negative (type II error). The probability of a false negative is given by P(b<b_{c}|M₁).
If b≥b_{c} then we reject the null hypothesis. Given that H₁ is in fact true, this is a true positive.

The probability of a true positive (and avoiding a false negative) is given by:

Power=P(b≥b_{c}|M₁) (8)

where the power is obtained by calculating the distribution of P(b|M₁) over the number of variant reads using equation (5), and obtaining the cumulative distribution to determine P(b≥b_{c}|M₁). This is the power of detecting a mutation using the test b≥b_{c} at a significance level P(b≥b_{c}|M₀). Thus, model M₀ is used to set b_{c} (which is the value such that we reject H0 if b≥b_{c} with a chosen significance level given by P(b≥b_{c}|M₀)), and model M₁ is then used to calculate the power to detect a mutation as being expressed if it is really expressed, using this value of b_{c}.

This approach may be used to determine whether a mutation is likely to be actually expressed (and if it is determined to be unlikely to be expressed, whether that is because the power to detect expression with the sequence data at hand is low), to detect mutations from RNA data (e.g. in particular for mutations that are only or advantageously detectable from RNA sequence data such as e.g. gene fusions and splice variants such as retained introns), and to determine the RNA sequencing depth that is necessary to be able to detect a particular mutation with a predetermined minimum power. For example, the approach may be used to determine the power to detect a mutation as being expressed given a plurality of candidate sequencing depths (resulting in a corresponding coverage value d and possible values of b depending on the expected ratio of expression of the normal and variant alleles and the tumour purity and genotype), and a depth that satisfies b>b_{c} at a significance level P(b≥b_{c}|M₀) and with a power above a predetermined value may be selected for use in sequencing a sample where a mutation is suspected to be present or expressed.

### Extension to Multi-Region Sequencing

The above approach may be repeated for each of a plurality of samples from the same subject, to determine the power to detect a tumour-specific mutation as being expressed in each sample.

### Results

The approach described above was exemplified using data in which a cell line (HCC1395, a human breast cancer cell line) was mixed with a reference cell line (matched normal cell line, the B lymphocyte derived cell line HCC1395L) using known proportions (10, 20, 50 and 100%) to model tumour samples with tumour purities of 10, 20, 50 and 100%. DNA and RNA sequence data were obtained for all samples. The genotypes and purity for each sample were estimated using Sequenza (Favero et al., 2015) and set to their maximum a posteriori (MAP) estimates. A baseline model as described above was used, assuming α=5,ε=0.001, and

p(θ|M₀)~Beta(9999,1)

p(θ|M₁)~Beta(1,1)

where α is the cell expression ratio,ε is the sequencing error rate, M₀ and M₁ are the null and alternative models, with different priors for the reference ratio θ. The variant and normal genotypes (G_{V} and G_{N}) and the tumour purity t were set to their MAP estimate.

The 100% sample can be used to assess the ground truth number of mutations, and whether they are expressed. Two replicate experiments were analysed in an identical manner.

For replicate 1: A total of 336 high confidence mutations in the data were identified in the 100% sample, and have at least 1 variant read in all the samples at the DNA level. Of these, 222 (66.07%) have more than 1 variant RNA-seq read in the 100% sample, and these are assumed to be the truly expressed mutations (true positives). The copy number and tumour purity could be estimated in all the dilutions (10, 20, 50, 100) for 333 out of the 336 mutations. 221 of these are expressed (true positives for which copy number and purity could be estimated, used as true positive set for these analyses), 112 are not expressed.

For replicate 2: A total of 333 high confidence mutations in the data which are AVID passing in the 100% sample, and have at least 1 variant read in all the samples at the DNA level. Of these, 220 (66.07%) have more than 1 variant RNA-seq read in the 100% sample, and these are assumed to be the truly expressed mutations (true positives). The copy number and tumour purity could be estimated in all of the dilutions (5, 10, 30, 100) for 332 out of the 333 mutations. 220 of these are expressed, 112 are not expressed.

To quantify the effect of tumour purity and genotypes in the model when calculating power to detect, the model was run on 5 different data sets (in each replicate):
Fixed genotypes, ground truth purity: the variant genotypes are assumed to be major copy number=1, minor copy number=1, tumour purity is the ground truth (i.e. the known % of tumour cells used).
Ground truth genotypes and purity: The variant genotypes are ‘ground truth’—estimate of major and minor copy numbers for each mutation at 100% purity, using Sequenza. The purity is the ground truth value.
Estimated genotypes, ground truth purity: The variant genotypes are derived from the estimate of major and minor copy numbers for each mutation as explained above, the purity is ground truth.
Ground truth genotypes, estimated purity: The variant genotypes are ‘ground truth’-estimates of major and minor copy numbers for each mutation at 100% purity, using modified Sequenza. The purity is estimated.
Fixed genotypes, estimated purity: The variant genotypes are assumed to be major copy number=1, minor copy number=1, the purity is estimated.

The purity and genotypes were jointly estimated using Sequenza (using Sequenza determine the probability that a tumour has a particular purity/ploidy value along a grid of possible values, and assigning genotypes for each variant given the associated LRR, BAF and ploidy value). The model was not run on estimated genotypes and estimated purity because part of the aim for the different data sets above was to investigate the effect of changing one variable at a time. However, given the results obtained, this is not expected to produce very different results.

In this example, α was set to 0.5 (assuming that the tumour and normal cells contribute equal amounts to the total expression at the locus).

For each mutation for which copy number and purity estimates were obtained in each of the series (5, 10, 30, 100), the following were calculated:
b_{c}: the critical value for the test of mutation expression: if the number of variant reads at that mutation is at least equal to b_{c} (b≥b_{c}), then the mutation is considered to be detected (i.e. expressed) and the null model of no expression is rejected;
alpha: the associated significance value (probability of false positive—detecting the mutation when it is not expressed) of the test, where alpha <0.05; note that as described above, bc is chosen such that alpha <0.05 but as bc is an integer, the actual value of alpha that satisfies the criterion may vary (and in particular is unlikely to be =0.05);
power: the power of detecting an expressed mutation (probability of rejecting null hypothesis when alternative is true).

For each variant the number of variant reads was compared to b_{c} to determine whether the variant is detected (b≥b_{c}), at this particular alpha.

For each sample, the false positive (FP) and false negative (FN) rates were also calculated., using the ground truth data (i.e. the 221 mutations that are known to be expressed in the 100% sample and the 112 mutations that are known not to be expressed in the 100% sample) When assessing the RNAseq data from a sample to decide whether a mutant is expressed, there are four different scenarios:
True positive: The mutated allele is expressed and expression is detected
False positive: The mutated allele is not expressed and expression is detected (Type I error)
True negative: The mutated allele is not expressed and expression is not detected
False negative: The mutated allele is expressed and expression is not detected (Type II error).

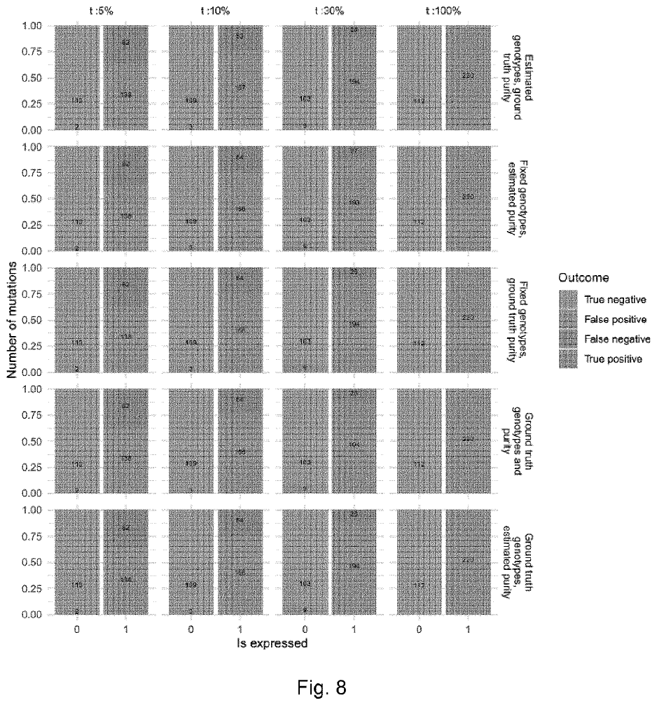

The results of these are shown on FIG. 8 (for replicate 2, the data for replicate 1 is similar and not shown for brevity) which shows that the FN rate (and to a smaller extent the FP) increases as the tumour purity decreases. The error rates for all of the data sets (different genotypes and purity sources) were relatively similar, but the estimated genotypes+ground truth purity had a slightly higher TP rate.

The average accuracy (true negative rate—TN, true positive rate—TP) over the 10, 20 and 50 purity series were, for replicate 1:
Estimated genotypes, ground truth purity: TN:0.9792; TP:0.7617
Fixed genotypes, estimated purity: TN:0.9792; TP:0.7602
Fixed genotypes, ground truth purity: TN: 0.9792; TP:0.7647
Ground truth genotypes and purity: TN: 0.9792; TP:0.7647
Ground truth genotypes, estimated purity: TN:0.9792; TP:0.7632

For replicate 2:
Estimated genotypes, ground truth purity: TN:0.9583; TP:0.7561
Fixed genotypes, estimated purity: TN:0.9583; TP:0.7530
Fixed genotypes, ground truth purity: TN: 0.9583; TP:0.7545
Ground truth genotypes and purity: TN: 0.9583; TP:0.7545
Ground truth genotypes, estimated purity: TN:0.9583; TP:0.7545.

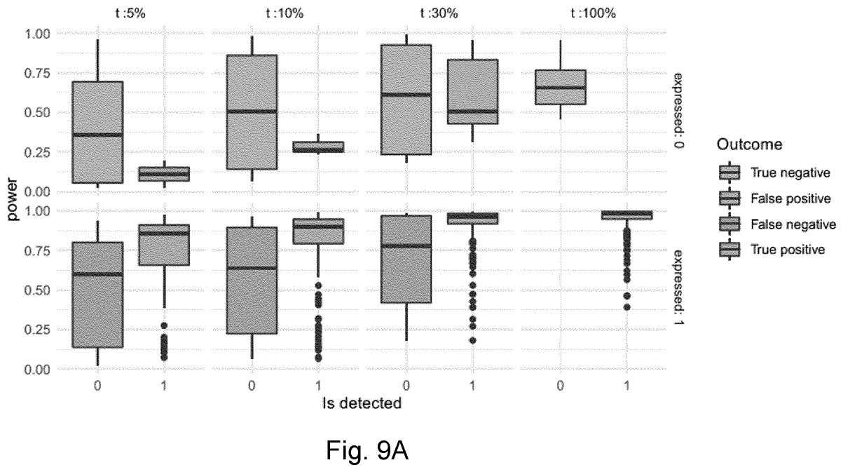

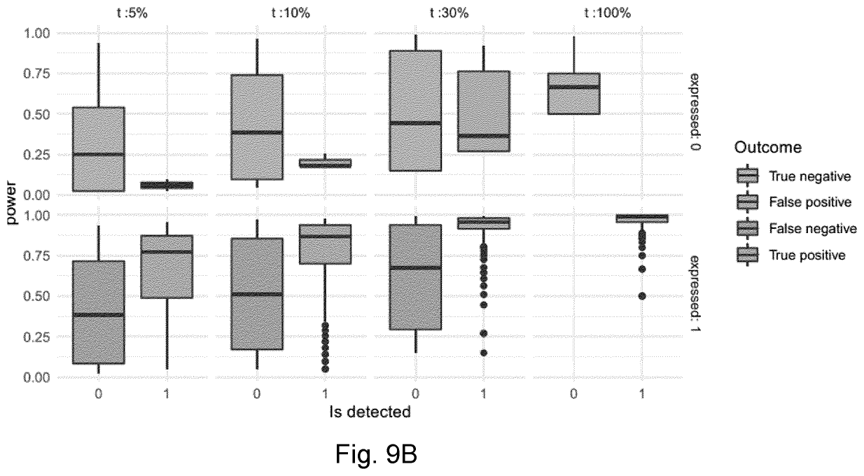

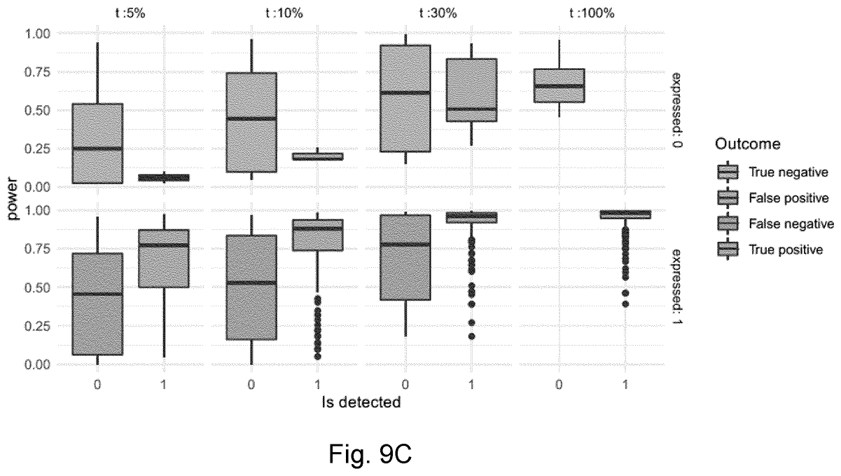

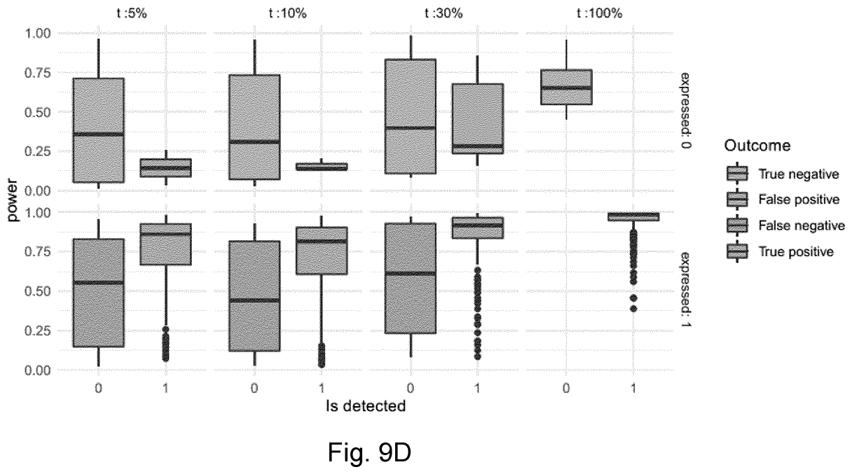

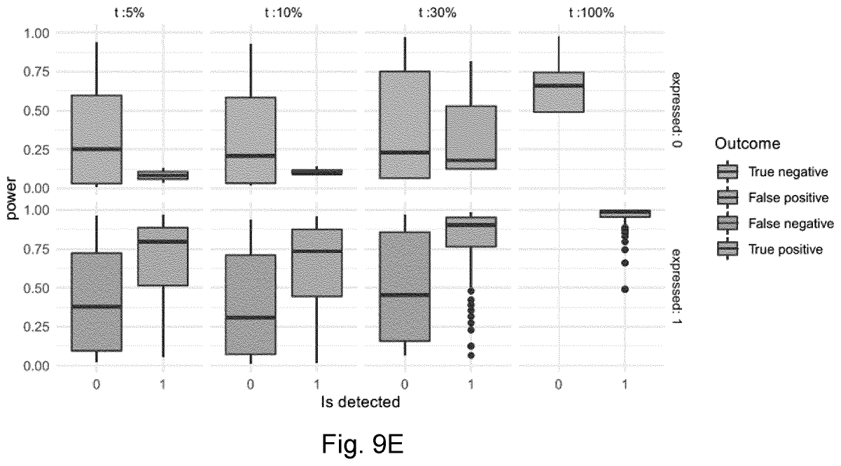

The TN was very similar across all cases as all use the same false positive rate (<0.05). The distribution of estimated power, grouped by the different outcomes (TP, FP, FN, TP) for each the tumour purities assessed is shown on FIGS. 9A-E for each of the data sets (replicate 2). The data for replicate 1 is similar and not shown for brevity. This shows that for the mutations that are truly expressed (FN+TP), the power is lower for false negatives (FN) than for true positives (TP), indicating that the method is able to identify cases where the data provides low power to detect a mutation (and hence absence of its detection does not necessarily mean absence of expression).

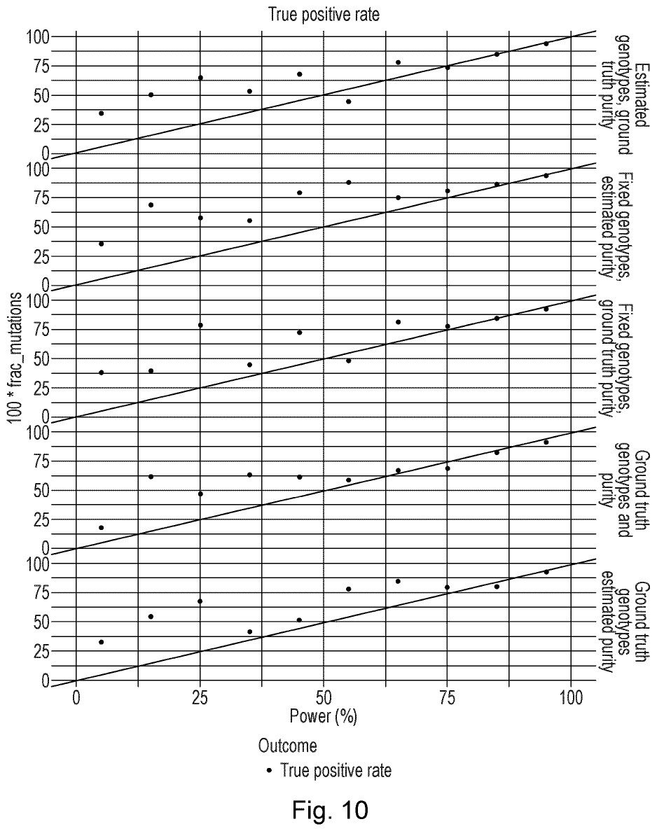

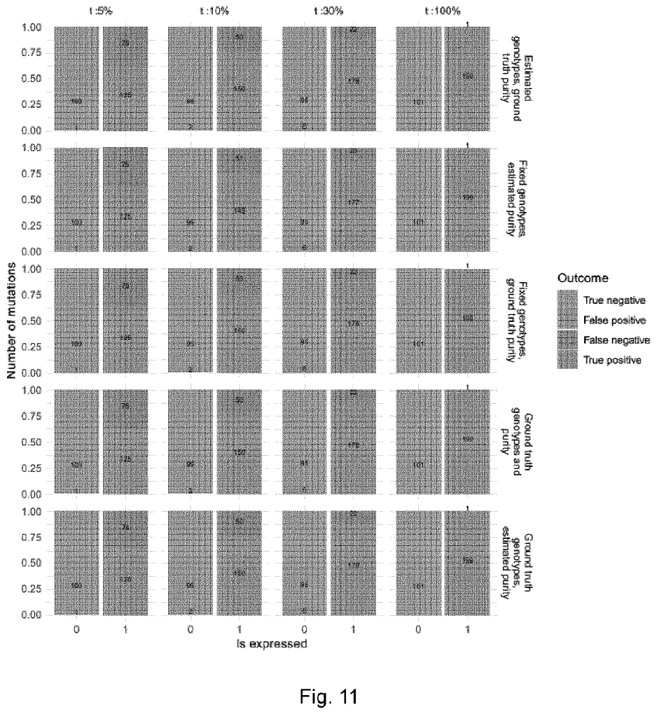

The mutations were then grouped by power values in bins of 10% of the power scale, and for each bin the fraction of true positives and false negatives was calculated. The results of this are shown on FIG. 10 (replicate 2). The data for replicate 1 is similar and not shown for brevity. This shows that the TP rate correlates with the power estimated by the model, in line with expectations. The solid line is the identity line (which is what would be expected for the model). This shows that the model may in some cases underestimate the true positive rate (as the real TP rate is in fact slightly above this line for at least the power bins under 60%). The mean squared error (MSE) and mean error (ME) of power estimate values are shown in Table 1.

TABLE 1
MSE and ME of power estimates

| Data - Replicate 1 | MSE | ME |
|---|---|---|
| Estimated genotypes, ground truth purity | 0.0288 | 0.1174 |
| Fixed genotypes, estimated purity | 0.0527 | 0.1750 |
| Fixed genotypes, ground truth purity | 0.0573 | 0.1772 |
| Ground truth genotypes and purity | 0.0337 | 0.1159 |
| Ground truth genotypes, estimated purity | 0.0297 | 0.1319 |
| Estimated genotypes, ground truth purity | 0.0475 | 0.1433 |
| Fixed genotypes, estimated purity | 0.0762 | 0.2202 |
| Fixed genotypes, ground truth purity | 0.0582 | 0.1600 |
| Ground truth genotypes and purity | 0.0406 | 0.1234 |
| Ground truth genotypes, estimated purity | 0.0545 | 0.1702 |

### Example 2—Estimation of the Alpha Parameter

This section introduces an approach to estimate the value of the parameter α (the fraction of total expression at the locus which is due to the normal cell population (e.g. TPMₙₒᵣₘₐₗ/(TPMₙₒᵣₘₐₗ+TPMₜᵤₘₒᵤᵣ)), used in Example 1. As mentioned in Example 1, the value of the parameter α is typically not known for a particular sample. In the simplest implementation, this can be set to a suitable default value, such as e.g. 0.5. This assumes that the normal and tumour cell populations contribute equally to the total expression signal (e.g. number of reads) at the locus. However, intuitively there should be a relationship between this parameter and tumour purity. Additionally, some loci may be overexpressed in tumour cells compared to normal cells. In this example, the inventors introduce a framework to estimate the value of α in situations where a plurality of samples with different purities are available. Other approaches can be used, for example when differential expression between the normal and tumour cells is known to occur and an estimate of this can be obtained (e.g. from previous data or databases).

### Methods

Relationship between TPM, purity t and α

The TPM value for gene i is given by:

$$T P {M}_{i} = \frac{{q}_{i}}{{Σ}_{i} {q}_{i}} 1 {0}^{6} \qquad ( 20 )$$

where

$${q}_{i} = \frac{{r}_{i}}{{l}_{i}}$$

is the number of reads that map to gene i (rᵢ) divided by the length of gene l (lᵢ). The superscripts T and N will be used to refer to the reads from the tumour cells and the reads from the normal cells, respectively. Assuming that:
the sample comprised n cells, with (1−t)n normal cells and tn tumour cells,

$${x}_{i}^{T}$$

reads for gene i are obtained for each tumour cell, and

$${x}_{i}^{N}$$

x reads for gene i are obtained for each normal cell;
   we have:

$${r}_{i}^{T} = t n {x}_{i}^{T} \qquad ( 21 a )$$

$${r}_{i}^{N} = ( 1 - t ) n {x}_{i}^{N} \qquad ( 21 b )$$

$$and :$$

$${q}_{i} = \frac{{r}_{i}^{T}}{{l}_{i}} + \frac{{r}_{i}^{N}}{{l}_{i}} = \frac{t n {x}_{i}^{T}}{{l}_{i}} + \frac{( 1 - t ) n {x}_{i}^{N}}{{l}_{i}} = t n {c}_{i}^{T} + ( 1 - t ) n {c}_{i}^{N} \qquad ( 22 )$$

where

$${c}_{i}^{T} = \frac{{x}_{i}^{T}}{{l}_{i}} and {c}_{i}^{N} = \frac{{x}_{i}^{N}}{{l}_{i}}$$

are the number of reads for gene i from each tumour cell and normal cell, respectively, normalised by gene length.

The total TPM in a sample with purity t is given by:

$${TPM}_{i} = \frac{{tnc}_{i}^{T} + ( 1 - t ) {nc}_{i}^{N}}{{Σ}_{i} {tnc}_{i}^{T} + ( 1 - t ) {nc}_{i}^{N}} {10}^{6} = \frac{{TPM}_{i}^{T}}{1 + \frac{( 1 - t ) {Σ}_{i} {c}_{i}^{N}}{t {Σ}_{i} {c}_{i}^{T}}} + \frac{{TPM}_{i}^{N}}{1 + \frac{t {Σ}_{i} {c}_{i}^{T}}{( 1 - t ) {Σ}_{i} {c}_{i}^{N}}} \qquad ( 23 )$$

where

$$TP {M}_{i}^{T} and {TPM}_{i}^{N}$$

are the TPM values for gene i for a tumour sample (assumed to have a tumour fraction t=1) and a normal sample (assumed to have a tumour fraction t=0), respectively.

We define:

$${α}_{i} = \frac{T P {M}_{i}^{N}}{T P {M}_{i}^{T} + T P {M}_{i}^{N}} \qquad ( 24 )$$

So we can rewrite the total TPM in terms of a for the gene:

$$T P {M}_{i} = ( TP {M}_{i}^{T} + T P {M}_{i}^{N} ) ( \frac{( 1 - {α}_{i} )}{1 + \frac{( 1 - t ) {Σ}_{i} {c}_{i}^{N}}{t {Σ}_{i} {c}_{i}^{T}}} + \frac{{α}_{i}}{1 + \frac{t {Σ}_{i} {c}_{i}^{T}}{( 1 - t ) {Σ}_{i} {c}_{i}^{N}}} ) \qquad ( 24 )$$

$$TP {M}_{i} = ( T P {M}_{i}^{T} + T P {M}_{i}^{N} ) ( \frac{( 1 - {α}_{i} ) t}{t + ( 1 - t ) \frac{{Σ}_{i} {c}_{i}^{N}}{{Σ}_{i} {c}_{i}^{T}}} + \frac{{α}_{i} ( 1 - t )}{( 1 - t ) + t \frac{{Σ}_{i} {c}_{i}^{T}}{{Σ}_{i} {c}_{i}^{N}}} ) \qquad ( {24}^{′} )$$

The quantity

$$\frac{{Σ}_{i} {c}_{i}^{N}}{{Σ}_{i} {c}_{i}^{T}}$$

is the ratio of (a) the number of reads normalised by gene length from a normal cell, relative to (b) the number of reads normalised by gene length from a tumour cell. These values are summed over all genes analysed and thus the ratio represents the total amount of RNA produced by tumour and normal cells. Assuming that this quantity is equal to 1 (i.e. assuming that the tumour and normal cells would produce similar amounts of total RNA, which is a reasonable assumption commonly made when looking at differential gene expression between different samples or conditions; note that the assumption does not mean that individual genes are expressed to the same extent between tumour and normal cells and this remains variable in the model), equation (24) simplifies to:

$$T P {M}_{i} = ( T P {M}_{i}^{T} + T P {M}_{i}^{N} ) ( ( 1 - {α}_{i} ) t + {α}_{i} ( 1 - t ) ) \qquad ( 25 )$$

$$TP {M}_{i} = ( T P {M}_{i}^{T} + T P {M}_{i}^{N} ) ( ( 1 - 2 {α}_{i} ) t + {α}_{i} ) \qquad ( {25}^{′} )$$

$$Such that :$$

$$if {α}_{i} = 0 then {TPM}_{i} = ( T P {M}_{i}^{T} + T P {M}_{i}^{N} ) t \qquad ( 26 )$$

$$if {α}_{i} = 1 then {TPM}_{i} = ( T P {M}_{i}^{T} + T P {M}_{i}^{N} ) ( 1 - t ) \qquad ( 27 )$$

The relationship between the total TPM for a gene (TPMᵢ), t and αᵢ is illustrated on FIG. 7 for an example with

$$T P {M}_{i}^{T} + T P {M}_{i}^{N} = 5 .$$

Thus, it is possible to estimate αᵢ by fitting a regression line to TPMᵢ values at a plurality of purities t. These could be obtained for example from multiple tumour samples from the same patient (e.g. where multiregion sequencing is available), or from multiple samples from a plurality of patients such as a plurality of patients with a particular cancer type recorded in a database such as TCGA (The Cancer Genome Atlas dataset, https://portal.gdc.cancer.gov/).

Indeed, we have:

$$at t = 0 , T P {M}_{i} ( 0 ) = ( T P {M}_{i}^{T} + T P {M}_{i}^{N} ) {α}_{i} \qquad ( 28 )$$

$$at t = 1 , T P {M}_{i} ( 1 ) = ( T P {M}_{i}^{T} + T P {M}_{i}^{N} ) ( 1 - {α}_{i} ) \qquad ( 29 )$$

and we can use a regression line to calculate TPMᵢ(0) and TPMᵢ(1) then use:

$$T P {M}_{i}^{T} + T P {M}_{i}^{N} = T P {M}_{i} ( 0 ) + T P {M}_{i} ( 1 ) \qquad ( 30 )$$

And from the slope g of the fitted line, using equation (25), get:

$${α}_{i} = \frac{1}{2} ( 1 - \frac{g}{T P {M}_{i}^{T} + T P {M}_{i}^{N}} ) \qquad ( 31 )$$

where TPMᵢ^{T} and TPMᵢ^{N} are estimated from a fitted regression line on total TPM as explained above, as the values of TPM provided by the regression model at t=0 and t=1.

### Results

In this example, the same data used in Example 1 (Replicate 2) was used to illustrate the use of a method as described herein with estimation of the a parameter (cell expression ratio) for each mutation. The baseline model described above was used, assuming a sequencing error rate ε=0.001 (note that this can in general be set depending on the expected error rate for the sequencing platform used), p(θ℄M₀)~Beta(9999,1) and p(θ|M₁)~Beta(1,1) (the priors for the reference ratio under the null and alternative models). The cell expression ratio was estimated for each mutation b a linear regression model using purity and gene expression values (transcripts per million, TPM), as explained above, across the titration series.

To quantify the effect of tumour purity and genotypes in the model when calculating the power to detect expression, the model was run on 5 different datasets, as in example 1 (i.e. ‘fixed genotypes, ground truth purity’, ‘ground truth genotypes, ground truth purity’, ‘estimated genotypes, ground truth purity’, ‘ground truth genotypes, estimated purity’, ‘fixed genotypes, estimated purity’). Only the mutations that had a copy number and a purity estimate in each of the samples of the titration series were used (301 mutations, of which 200 are expressed—more than 1 variant RNA-seq read in the 100% sample).

For each mutation, the following values were calculated:
b_{c}: the critical value for the test of mutation expression: if the number of variant reads at that mutation is at least equal to b_{c} (b≥b_{c}), then the mutation is considered to be detected (i.e. expressed) and the null model of no expression is rejected;
alpha: the associated significance value (probability of false positive—detecting the mutation when it is not expressed) of the test, where alpha <0.05 as explained above;
power: the power of detecting an expressed mutation (probability of rejecting null hypothesis when alternative is true).

For each mutation the number of variant reads was compared to b_{c} to determine whether the variant is detected (b≥b_{c}), at this particular alpha.

For each sample, the false positive (FP) and false negative (FN) rates were also calculated. When assessing the RNAseq data from a sample to decide whether a mutant is expressed, there are four different scenarios:
True positive: The mutated allele is expressed and expression is detected
False positive: The mutated allele is not expressed and expression is detected (Type I error)
True negative: The mutated allele is not expressed and expression is not detected
False negative: The mutated allele is expressed and expression is not detected (Type II error)

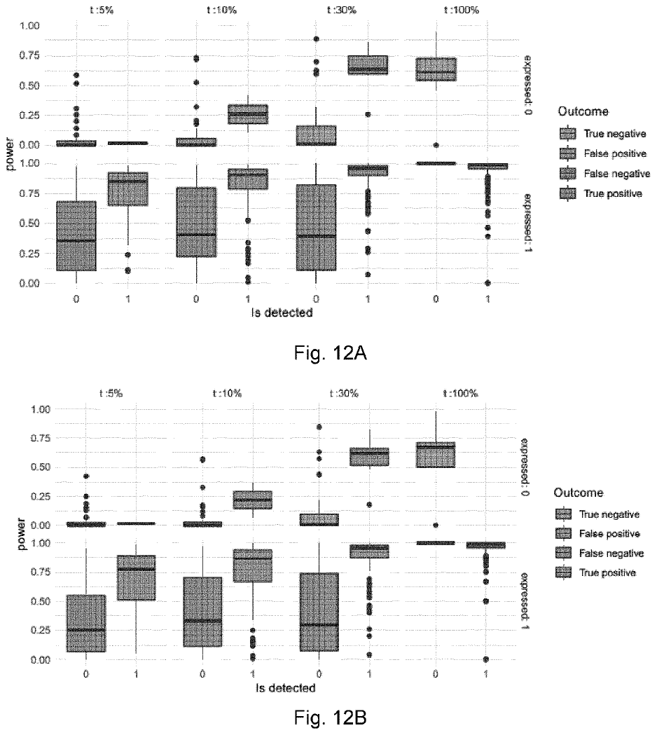

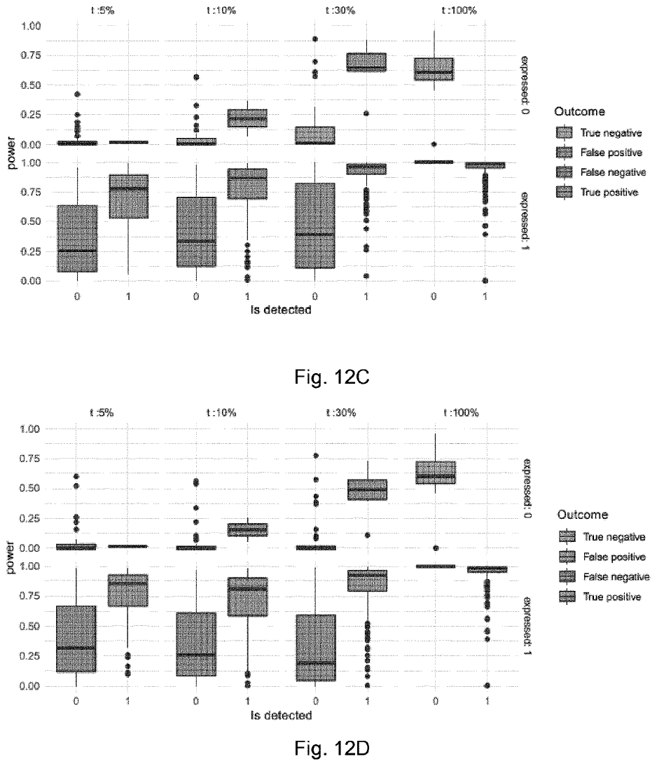

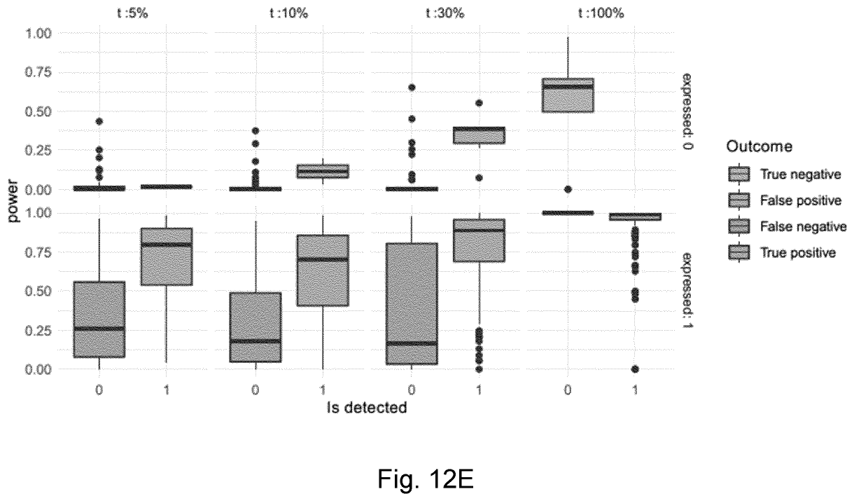

The results of this are shown on FIG. 11 and Table 2. As expected, both types of error increased as purity decreased. The error rates were similar in all datasets, although the ground truth genotypes, estimated purity had a slightly higher TP rate. The power to detect a mutation provides a indication of whether we can have confidence in calling whether a mutated allele is expressed. The distribution of estimated power from the model, grouped by different outcomes (TP, FN, FP, TP) and separated by tumour purity is shown on FIG. 12A-E (for each of the 5 datasets). This shows that for the mutations that are truly expressed, the power is lower for false negatives that for true positives.

TABLE 2
Average error rates over the 10, 20 and 50% purity samples

| Dataset | TN rate | TP rate |
|---|---|---|
| Estimated genotypes, ground truth purity | 0.9703 | 0.7550 |
| Fixed genotypes, estimated purity | 0.9703 | 0.7517 |
| Fixed genotypes, ground truth purity | 0.9703 | 0.7550 |
| Ground truth genotypes and purity | 0.9703 | 0.7550 |
| Ground truth genotypes, estimated purity | 0.9703 | 0.7567 |

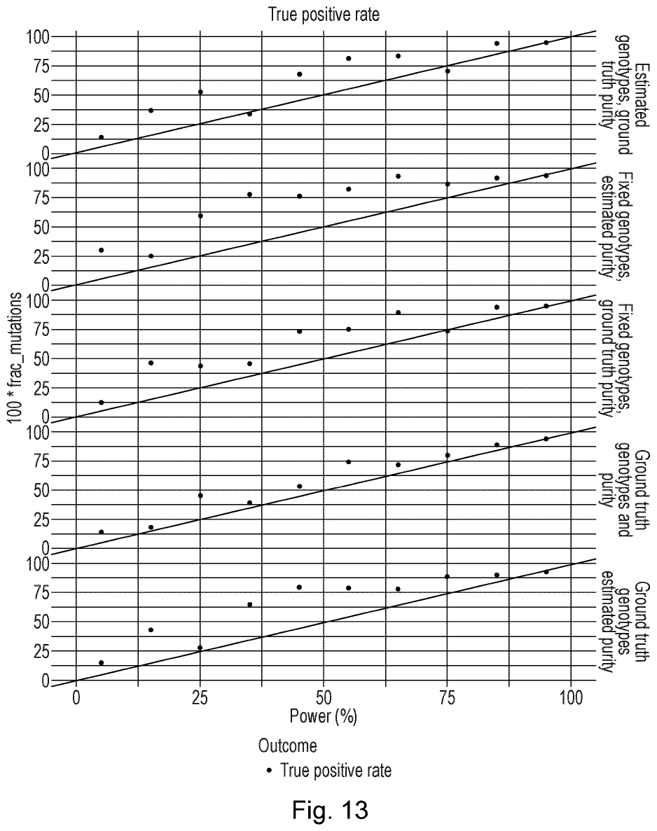

The mutations were then grouped by power values (using bins of 10%). For each bin, the fraction of true positives and the fraction of true negatives were calculated. The results of this are shown on FIG. 13, which shows that the TP rate correlates with the power estimated by the model, as expected. The mean squared error was calculated based on the TP rate and the average estimated predicted power per bin:
Estimated genotypes, ground truth purity: mse=0.0293, me=0.1278
Fixed genotypes, estimated purity: mse=0.0646, me=0.2158
Fixed genotypes, ground truth purity: mse=0.0334, me=0.1476
Ground truth genotypes and purity: mse=0.0098, me=0.0754
Ground truth genotypes, estimated purity: mse=0.0396, me=0.1611

This data shows that estimation of the a parameter improves the correlation between estimated power and TP rate. Therefore, when samples of various purities are available, estimation of a using linear regression can improve the accuracy of the model in detecting allele specific expression.

### Example 3—Probability that a Mutation is Expressed

This section introduces a unified model to determine whether a mutation is expressed or not, using the statistical hypothesis framework introduced above to estimate the power of detecting an expressed mutation. Example 1 describes models of variant expression and no variant expression using a binomial likelihood for the number of variant reads at a site with coverage d (Equation (3)) (illustrated on FIG. 4). Example 2 illustrates the process of estimation of the a parameter (cell expression ratio) in the model of Example 1. In the present example, a model to determine the probability that a mutation is expressed or not is presented, as illustrated on FIGS. 5A and 5B, respectively for a single sample (e.g. a single tumour region) and a plurality of samples (e.g. a plurality of tumour regions), based on the framework in Example 1. This approach advantageously combines the information in the model of Example 1 with prior information about a mutation being expressed, within a Bayesian framework. Additionally, the approach provides a single, interpretable probability of a tumour-specific mutation being expressed. This can be used for example to rank or otherwise prioritise tumour specific mutations for further analysis, for use as therapeutic targets, for use as diagnostic markers, etc. As this is within the context of a Bayesian framework, the probability advantageously combines information from the data available (RNA sequence data) in the form of the likelihood of the data under different models (expression/not expression of the variants), as well as information about prior beliefs of whether the mutation is expressed (hence the reference to a “unified” model, combining likelihood and prior probability of expression).

### Methods

### Probabilistic Measure of Whether a Variant is Expressed

Denoting expression of the variant allele by E (E=1 means the variant is truly expressed, E=0 means the variant is not expressed), the prior on θ (the reference ratio=reference read count/total read count, the balance between reference and total allele expression) is conditional on E (Equation (4)) such that the likelihood for the model is:

$$P ( b , d \vert {M}_{0} ) = P ( b , d \vert E = 0 , t , α ) = {\sum }_{g = 1}^{G} \int P ( b , d \vert {G}_{g} , θ , α , t ) p ( θ \vert E = 0 ) p ( {G}_{g} ) d θ \qquad ( 5 ’ )$$

$$P ( b , d \vert {M}_{1} ) = P ( b , d \vert E = 1 , t , α ) = {\sum }_{g = 1}^{G} \int P ( b , d \vert {G}_{g} , θ , α , t ) p ( θ \vert E = 1 ) p ( {G}_{g} ) d θ$$

where we integrate over all the possible genotypes G and θ. Note that equations (5) and (5′) are equivalent.

Assume that E is a Bernoulli variable with parameter ρ, such that p(E=1)=ρ, p(E=0)=(1−ρ).

This is the prior probability of E, i.e. the prior probability of the mutation being truly expressed (E=1) or truly not expressed (E=0). We can also place a hyperprior on ρ such that we have:

p(E|ρ)=Bernoulli(E|ρ) (9a)

p(ρ)=Beta(ρ|α_{ρ},β_{ρ}) (9b)

As will be described further below, because the probability of a mutation being expressed is obtained by integrating over ρ, the values of α_{ρ},β_{ρ} are not in fact used and the model only needs to have the mean of the distribution (which assuming the above distribution, would be provided by μ_{ρ}=α_{ρ}/(α_{ρ}+β_{ρ})). The value of μ_{ρ} can be set using prior knowledge of whether the mutation is likely to be expressed in the sample. This can be based on e.g. prior knowledge about the type of mutations, the type of cancer, the gene in which the mutation is located, etc.

Based on the sequence data from a sample, it is possible to calculate the joint posterior distribution on E and ρ:

$$P ( E , ρ \vert b , d , α , t ) = \frac{P ( b , d \vert α , t , E ) p ( E \vert ρ ) p ( ρ )}{{\sum }_{e = 0 , 1} \int P ( b , d \vert α , t , E = e ) p ( E = e \vert ρ ) p ( ρ ) d ρ} \qquad ( 10 )$$

We want to calculate the marginal distribution on E:

P(E=1|b,d,α,t)∝∫P(b,d|α,t,E=1)p(E=1|ρ)p(ρ)dρ (11)

Defining the likelihood ratio of model M₁ over model M₀ as r, which is given by equation (12):

$$r = \frac{P ( b , d \vert α , t , E = 1 )}{P ( b , d \vert α , t , E = 0 )} \qquad ( 12 )$$

we can rewrite equation (11) to obtain equation (13):

P(E=1|b,d,α,t)∝∫P(b,d|α,t,E=1)p(E=1|ρ)p(ρ)dρ

$$P ( E = 1 \vert b , d , α , t ) = \frac{\int P ( b , d \vert α , t , E = 1 ) ρ p ( ρ ) d ρ}{\begin{matrix}\int ( P ( b , d \vert α , t , E = 0 ) ( 1 - ρ ) p ( ρ ) + \\ P ( b , d \vert α , t , E = 0 ) ρ p ( ρ ) ) d ρ\end{matrix}} \qquad ( 13 )$$

$$P ( E = 1 \vert b , d , α , t ) = \frac{P ( b , d \vert α , t , E = 1 ) {μ}_{p}}{\begin{matrix}P ( b , d \vert α , t , E = 0 ) ( 1 - {μ}_{p} ) + \\ P ( b , d \vert α , t , E = 1 ) {μ}_{ρ}\end{matrix}}$$

$$P ( E = 1 \vert b , d , α , t ) = \frac{{μ}_{ρ}}{( \frac{1 - {μ}_{ρ}}{r} + {μ}_{ρ} )}$$

Where μ_{ρ} is the mean of the Beta distribution (or any other suitable distribution) over ρ, i.e. μ_{ρ}=α_{ρ}/(α_{ρ}β_{ρ}). Equation (13) shows that the posterior over E=1 is dependent on the likelihood ratio r, and the mean of the Beta distribution over ρ. Indeed, since we have integrated over ρ, this model only uses a point estimate of ρ (i.e. the mean).

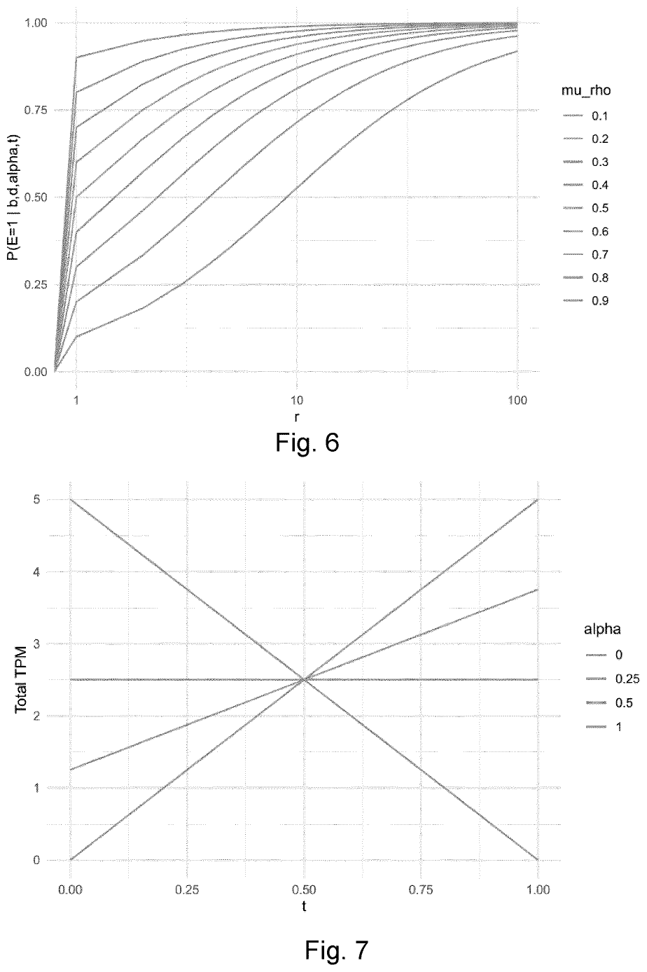

Equation (13) can be rewritten to further the intuitive interpretation of the model as equation (14), such that the posterior on E=1 is given by a logistic function, whose argument is the log likelihood ratio of the two outcomes of E plus the log prior odds (as illustrated on FIG. 6):

$$P ( E = 1 \vert b , d , α , t ) = \frac{{μ}_{ρ}}{( \frac{1 - {μ}_{ρ}}{r} + {μ}_{ρ} )} \qquad ( 14 )$$

$$P ( E = 1 \vert b , d , α , t ) = \frac{1}{( \frac{1 - {μ}_{ρ}}{r {μ}_{ρ}} + 1 )}$$

$$P ( E = 1 \vert b , d , α , t ) = \frac{1}{exp ( - log ( r \frac{{μ}_{ρ}}{1 - {μ}_{ρ}} ) + 1}$$

$$P ( E = 1 \vert b , d , α , t ) = logistic ( log ( r ) + log ( \frac{{μ}_{ρ}}{1 - {μ}_{ρ}} ) )$$

FIG. 6 shows that when a prior indicating a low chance of the variant being expressed (low μ_{ρ}), a lot of evidence (high likelihood ratio r) is needed to push the posterior probability to high values (high probability of expression). The figure also shows that in cases where there is a low power to detect variant alleles and low evidence of expression (likelihood ratio close to 1, the null and alternative models are similar) the probability will be close to the prior. With high evidence (number of reads higher than the critical value, high likelihood ratio), the probability that the variant is expressed is higher.

Thus, in cases where:
there is a high power to detect the variant allele and the likelihood supports that there is no expression of the mutant allele, then b<b_{c} and there is strong evidence in favour of M₀, leading to a very small r. As r approaches 0 (r→0), then P(E=1|b,d,α,t)=0.
There is a high power to detect the variant allele and the likelihood supports that there is expression of the mutant allele, then b≥b_{c} and there is strong evidence in favour of M₁, leading to a very large r. As r approaches (r→∞), then P(E=1|b,d,α,t)=1.
There is a low power to detect the variant allele and the likelihood supports that there is no expression of the mutant allele, then b<b_{c} and the data is equally likely under both M₀ and M₁, such that r approaches 1 (r=1) and P(E=1|b,d,α,t)=μ_{ρ}.
There is a low power to detect the variant allele and the likelihood supports that there is expression of the mutant allele, then b≥b_{c} and it is possible that there will be values of b where r is very high, and as r approaches (r→∞), then P(E=1|b,d,α,t)=1. This is a desired behaviour as where there is strong evidence that the mutation is expressed, even when the power to detect the mutation is low one would want the probability to reflect a high confidence in there being expression of the variant allele (E=1).

If there is no coverage for a mutation, i.e. d≈0, this could be due to:
a biological cause such as the gene harbouring the mutation has been downregulated, and the mutation is likely to not be expressed. This creates a scenario where we effectively lack data and thus the likelihood is missing. In a Bayesian model, we would fall back to the prior (i.e. the posterior would be equal to the prior). This intuitively doesn't make sense in the present context as one would expect that low/lack of gene expression means that there is a low chance of the variant allele being expressed (i.e. d approaching 0, i.e. lack of data, is informative as to whether the variant allele—and the reference allele—is likely to be expressed), because there is no formal parameter to model the gene expression process.
A technical issue, such that there is no data, and we can't infer whether mutation was expressed or not.

In order to resolve this, we can use prior knowledge about whether the gene is expressed (in reference data, e.g. in normal tissue, tumour tissue, tumour of the same type, etc.) or not to determine whether the absence of reads for the locus is a technical issue. If we know that the gene is highly expressed in the sample, and there is no read coverage at the particular position of the variant, the absence of reads is likely to be a technical issue, and μ_{ρ}=0.5. If we know that the gene should be lowly expressed, and there is no read coverage, we can likely set μ_{ρ}<0.5 (because if the gene should not be expressed then the mutation is also unlikely to be expressed).

A simple solution to the problem is to set a threshold of gene expression (e.g. TPM>1) of the gene that harbours the variant. The TPM is the number of reads mapped to the gene, divided by a scaling factor that is the total number of reads in the sample divided by 1,000,000, and normalised by the length of the gene. If the gene does not satisfy this condition (i.e. TPM=0 or 1) in the sample analysed, then μ_{ρ}<0.5.

If the gene is above this, then μ_{ρ} can be set to 0.5 or higher. The threshold used may be set to any predetermined value, preferably a low value in order to capture genes with low expression. Alternatively, the threshold may be set to a value that results in good performance of the model to detect genes with low expression, or to a value that reflects the TPM value below which a gene is unlikely to be expressed, using reference expression data such as e.g. expression data from a cohort of samples. When using a plurality of samples, the condition may be verified for each sample individually and the results over the multiple samples may be combined to determine whether the gene satisfies the condition. For example, the condition may be required to be verified in each sample or in a majority of the samples.

In the results shown below, the prior was set to μ_{ρ}=0.5 unless the read count was 0 and the gene was expected to be expressed, in which case the prior was set to a lower value of μ_{ρ}=0.05.

Note that this is a key adaptation of a “traditional” Bayesian approach to the particular biological problem under investigation. Indeed, in a traditional Bayesian framework when there is no data, the posterior should be equal to the prior (there is no likelihood to inform the decision away from the prior). Thus, it is normally the desired and expected behaviour of a Bayesian framework that the probability of a gene being expressed should fall back to e.g. a probability of p=0.5 (i.e. 50-50 chances of being expressed vs not being expressed) in the absence of data. However, the present inventors have identified that in the present context, the lack of data (no reads at the locus under investigation) may be informative in itself because it may be due to there being no gene expression at the locus. If there is no gene expression at the locus, then it is more likely than not that the variant is not expressed. Thus, the present inventors have adapted a simple Bayesian framework with a single prior to instead include a prior that has multiple possible values including at least a first value if there is evidence that the locus is expressed, and a second value if there is not enough evidence that the locus is expressed.

### Extension to Multi-Region Sequencing

For multi-region sequencing, when we have sequencing data from multiple regions of the tumour, the model can be extended to use the multiple sources of information. Suppose that there are S regions, and subscripts indicates the s^{th} region. For a locus, for each of the S regions, the likelihood of expression/no expression is given by:

P(bₛ,dₛ|M₀)=P(bₛ,dₛ|E=0,tₛ,α)=∫∫P(bₛ,dₛ|G,θ,α,tₛ)p(θ|E=0)p(G)dθdG

P(bₛ,dₛ|M₁)=P(bₛ,dₛ|E=1,tₛ,α)=∫∫P(bₛ,dₛ|G,θ,α,tₛ)p(θ|E=1)p(G)dθdG (5″)

We expect a and G to be common to all regions. The posterior probability of E=1 is given by (equation 14′):

$$P ( E = 1 \vert \{ {b}_{1} , … {b}_{s} \} , \{ {d}_{1} , … {d}_{s} \} , \{ {t}_{1} , … {t}_{s} \} , α ) ∝ \int {\prod }_{s = 1}^{S} P ( {b}_{s} , {d}_{s} \vert α , {t}_{s} , E = 1 ) p ( E = 1 \vert ρ ) p ( ρ ) d ρ = \frac{{μ}_{ρ} {\prod }_{s = 1}^{S} P ( {b}_{s} , {d}_{s} \vert α , {t}_{s} , E = 1 )}{\begin{matrix}( 1 - {μ}_{ρ} ) {\prod }_{s = 1}^{S} P ( {b}_{s} , {d}_{s} \vert α , {t}_{s} , E = 0 ) + \\ {μ}_{ρ} {\prod }_{s = 1}^{S} P ( {b}_{s} , {d}_{s} \vert α , {t}_{s} , E = 1 )\end{matrix}} = \frac{{μ}_{ρ}}{( \frac{1 - {μ}_{ρ}}{{\prod }_{s = 1}^{S} {r}_{s}} + {μ}_{ρ} )} = logistic ( {\sum }_{s = 1}^{S} log ( {r}_{s} ) + log ( \frac{{μ}_{ρ}}{1 - {μ}_{ρ}} ) ) \qquad ( 14 ’ )$$

where rₛ is the likelihood ratio of M₁ to M₀ given the data covering the mutation in region s:

$${r}_{s} = \frac{P ( {b}_{s} , {d}_{s} \vert E = 1 , {t}_{s} , α )}{P ( {b}_{s} , {d}_{s} \vert E = 0 , {t}_{s} , α )} \qquad ( 12 ’ )$$

### Results

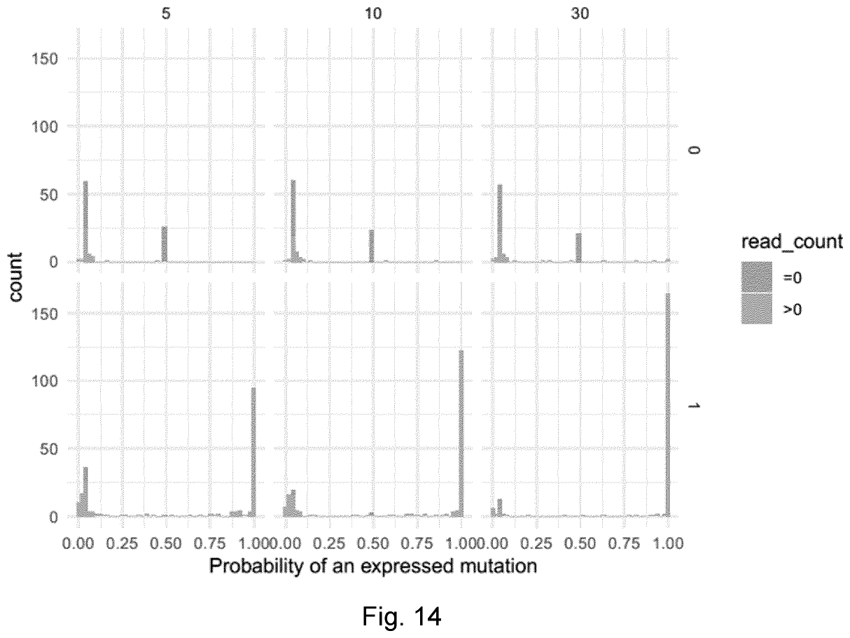

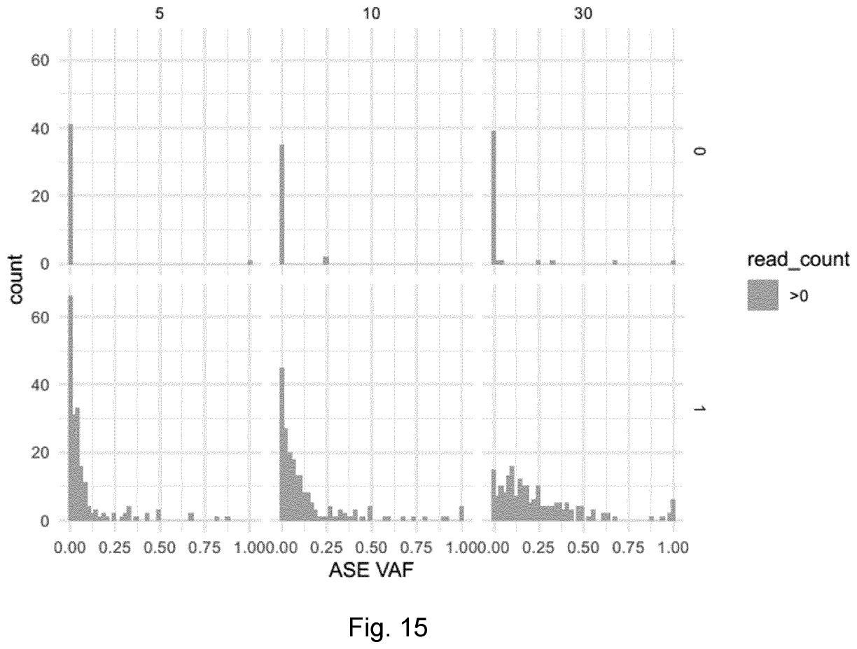

The model described in the methods section can be used with a default value for a or with an estimated value. The latter is illustrated here. The same data used in Example 2 was used, with the same parameters for the sequencing error rate E and the priors for θ under the null and alternative models. The posterior probabilities of each mutation being expressed were calculated as explained in the methods section above, for the data described in Example 1 (replicate 2). The distribution of these probabilities, split by ground truth purity (5, 10, 30%) and ground truth expression status (expressed at the bottom, or not at the top) is shown on FIG. 14. These were compared to a simpler model where the probability of a mutation being expressed is given by its variant allele frequency (i.e. the proportion of reads with the variant, b/d). The distribution of these simpler VAF model probabilities is shown on FIG. 15.

FIG. 14 shows that in the present method, the mutations with total read count d=0 default to the prior (in this case μ_{ρ}=0.5 if the gene is expected to be expressed—i.e. TPM>1, and μ_{ρ}=0.05 if the gene is expected to be lowly expressed—TPM≤1—where expectation of expression was determined based on calculated TPM values for each of the data sets in the purity series), as expected. For the mutations that did not have a total read count d=0, the prior was set to 0.5 for all mutations. The mutations that were truly not expressed (top row) are assigned very low probabilities (see peak near 0), and the mutations that were truly expressed (bottom row) are mostly assigned high probabilities (see peak near 1), with only a few false negative mutations with read counts >0 and probabilities near the lower end of the scale.

FIG. 15 shows that by contrast the “probabilities” derived from the VAF are much more spread out for the mutations that are in fact expressed—with many mutations having very low VAF, such that there is no natural threshold to identify mutations as expressed or not. Thus, using this model would either result in very high false positives or very high false negatives. In other words, comparing FIGS. 14 and 15 shows that the method described here is able to integrate the data within a biologically grounded model to push the confidence in expression vs no expression from a prior belief to confident determinations of expression vs no expression, unless the data genuinely does not provide enough information to make such a determination.

Having calculated the probability of expression for each mutation at each of 3 different purities (5, 10 and 30%), this was used to quantify a plurality of performance metrics:
ROC curve: TP and FP rates using varying thresholds for calling a mutation as expressed/not expressed: this shows the effect of the decision threshold on the TP and FP rates, and allows to calculate the AUC (Area Under the ROC Curve), and the threshold that gives the best TP rate with a FP rate below 0.05;
The AUC: this represents the probability that a random positive example (mutation that is truly expressed) is ranked higher (has a greater probability of being expressed) than a random negative example (mutation that is truly not expressed);
Calibration curve: the AUC and ROC demonstrate a model's ability to differentiate between positive and negative cases, but we would like to assess whether the probabilities used to predict whether a mutation is expressed are calibrated correctly in that they reflect the true likelihood, i.e. out of all the mutations which have been assigned a 50% probability of being expressed, are 50% of them expressed? The calibration of the model was tested by binning the data by probability (10% bins) and for each bin, calculating the mean probability of all the mutations in the bin, as well as the fraction of positives;
Brier score: the Brier score is used to evaluate the accuracy of the probabilistic predictions and is given by mean(True label(0 or 1)—probability²);
Predictive mean square error: using the calibration curve, the mean squared error over bins was calculated: mean((Fraction of TP—mean probability)²).

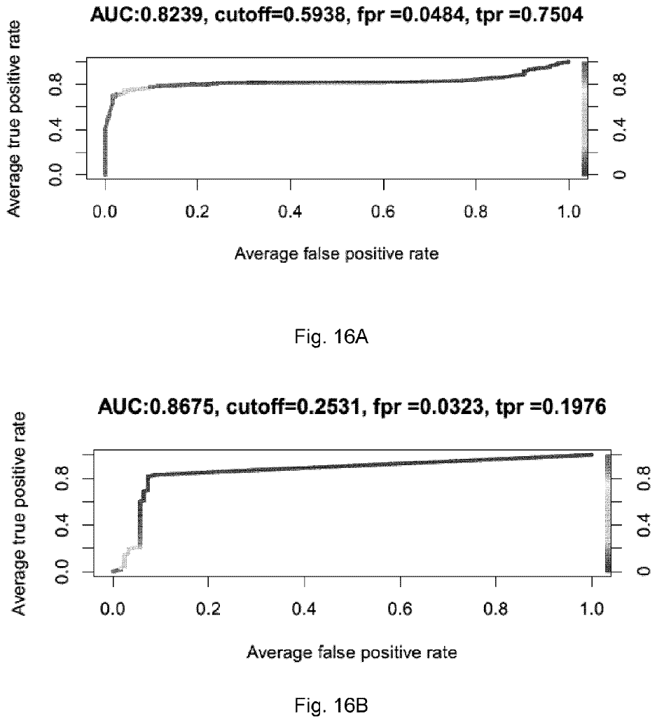

These metrics were calculated for the model described herein and for the simpler VAF model where the VAF is used as the probability of a mutation being expressed. In the first instance, these were calculated removing edge cases where the total read depth is 0. The ROC curves for the present and simpler VAF models are shown on FIGS. 16A and B, respectively. The cutoff values were chosen such that the TPR was maximised while keeping FPR<0.05. These show that although the simpler VAF model has a very slightly higher AUC, the present model has a better performance than the VAF model towards the left of the curve, where the simpler VAF model TPR drops as the FPR goes below around 9%, whereas the performance of the present model remains good throughout. This is due to the low frequency mutations which are expressed being misclassified. The present model achieved a TPR of 75% and a FPR of 5% on this data set. This indicates that the present model provides very informative predictions (any AUC over 50% is better than random guess). As discussed further below, the present model can likely be improved by further calibration, particularly by adapting the choice of prior. While a model using the VAF (proportion of reads with the variant) may appear informative in the particular cases shown, such a model has severe limitations. For example, it provides an estimate that is not comparable across samples, making it impossible to make reliable, reproducible and verifiable decisions for the same patient. Indeed, a VAF of 10% in a 5% purity sample would provide a strong indication that the variant is expressed, whereas a VAF of 10% in a 90% purity sample would be much more likely to be a false positive due to e.g. sequencing error. Additional factors such as genotype could also influence whether the same VAF is considered reliable or not. In other words, the use of the VAF as an indicator of likelihood of expression does not account for the influence of genotype and tumour purity, thereby making it an unreliable indicator of whether a variant is expressed.

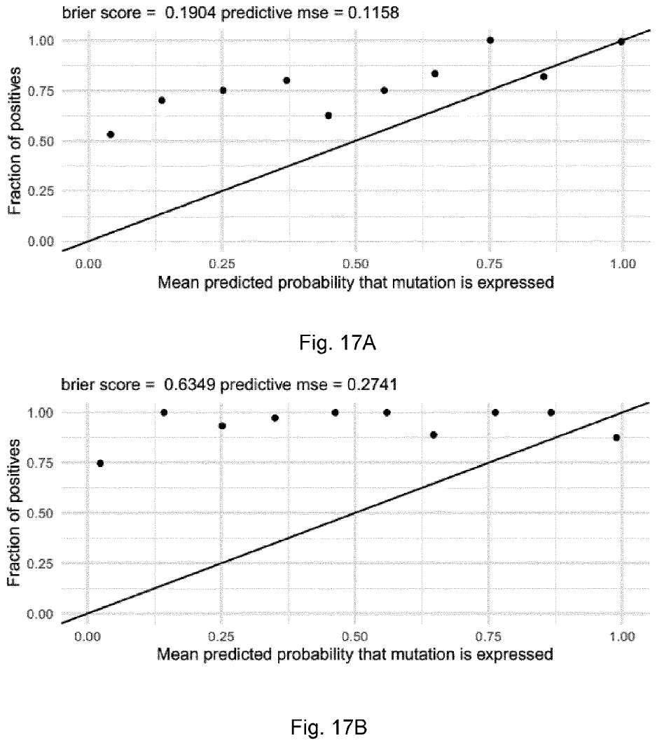

The calibration curves are shown on FIGS. 17A and B, respectively for the present model and the simpler VAF model. These show that both models are not as well calibrated for lower predictive probability estimates, and underestimate the true probabilities. However, the Brier score for the present model is relatively good. This could be further improved with additional model calibration.

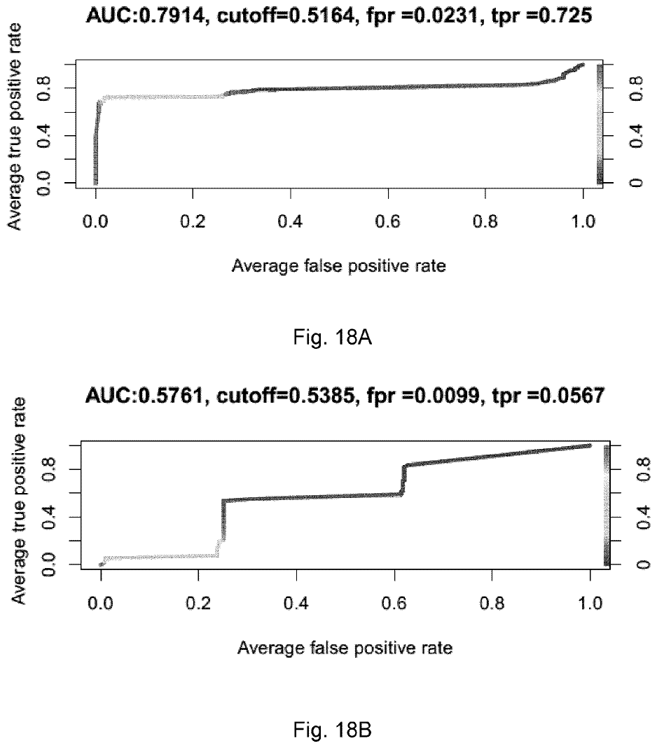

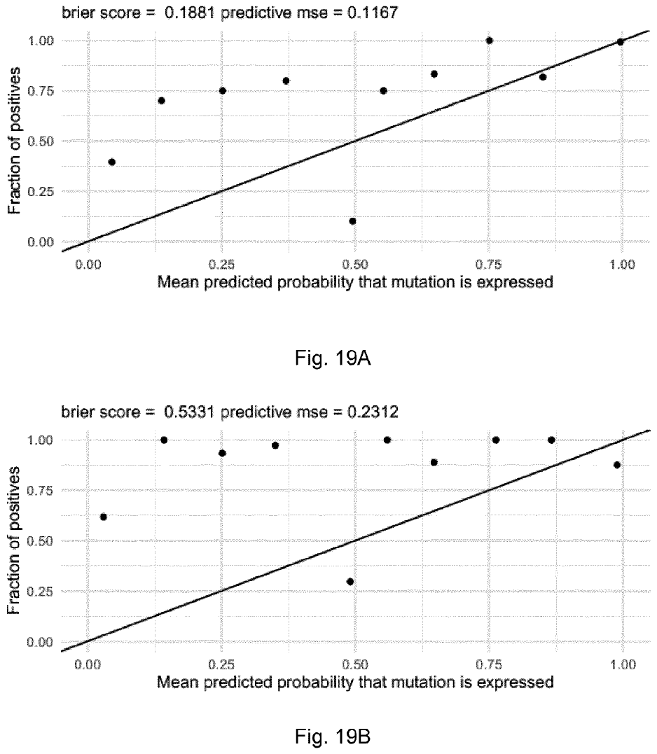

Including the edge cases (where the total read depth is 0), the simpler VAF model cannot be used as the VAF is not defined when there are no reads. Thus, this model was adapted by, for any mutation with d=0, using a threshold for gene expression (TPM>1) and setting p=0.05 below this threshold, and p=0.5 otherwise. The ROC curves are shown on FIGS. 18A and 18B, respectively for the present model and the simpler VAF model, and the calibration curves are shown on FIGS. 19A and 19B, respectively for the present model and the VAF model. As can be seen on FIGS. 18B and 19B, because the VAFs are not well calibrated probabilities, they cannot integrate the prior assumptions (used in the edge cases to infer probability of expression), and the simpler VAF model performs poorly.

By contrast, with edge cases the calibration curve for the present model is slightly better than without edge cases (see FIG. 18A), with a Brier score of 18.8%. The prediction at 50% overestimates the true probability of expression (see FIG. 19A). A large majority of mutations assigned to 50% are edge cases (no reads). This suggests that the probability of expression (for edge cases) could be improved by further calibration for this data set.

The present examples introduce and demonstrate the use of an approach to infer the probability that a mutation is expressed based on RNA sequence data. This uses a probabilistic framework that incorporates tumour purity, genotype and ploidy estimates, and accounts for variable expression both at the allele level(allele specific expression) and at the cell level (cell specific expression) to model the likelihood of detecting variant reads. The cell specific expression a was identified to pay a key role in the accuracy of the power estimate output by the model, and a way to estimate this when data is available to do this was also introduced and demonstrated. The performance of the approach using cell line data was finally evaluated, showing a good classification performance (accuracy of classification of mutations as expressed vs not expressed).

### REFERENCES

Gartner, Jared J., et al. 2021. “A Machine Learning Model for Ranking Candidate HLA Class I Neoantigens Based on Known Neoepitopes from Multiple Human Tumor Types.” Nature Cancer 2 (5): 563-74
Carter S L, Cibulskis K, Helman E, McKenna A, Shen H, Zack T, Laird P W, Onofrio R C, Winckler W, Weir B A, Beroukhim R, Pellman D, Levine D A, Lander E S, Meyerson M, Getz G. Absolute quantification of somatic DNA alterations in human cancer. Nat Biotechnol. 2012 May; 30(5):413-21.
Vanessa Jurtz, Sinu Paul, Massimo Andreatta, Paolo Marcatili, Bjoern Peters and Morten Nielsen. NetMHCpan-4.0: Improved Peptide-MHC Class I Interaction Predictions Integrating Eluted Ligand and Peptide Binding Affinity Data. J Immunol Nov. 1, 2017, 199 (9) 3360-3368.
Langmead, B., Trapnell, C., Pop, M. et al. Ultrafast and memory-efficient alignment of short DNA sequences to the human genome. Genome Biol 10, R25 (2009).
Lundegaard C, Lamberth K, Harndahl M, Buus S, Lund O, Nielsen M. NetMHC-3.0: accurate web accessible predictions of human, mouse and monkey MHC class I affinities for peptides of length 8-11. Nucleic Acids Res. 2008 Jul. 1; 36(Web Server issue):W509-12.
McGranahan, N., Furness, A. J., Rosenthal, R., Ramskov, S., Lyngaa, R., Saini, S. K., Jamal-Hanjani, M., Wilson, G. A., Birkbak, N. J., Hiley, C. T., Watkins, T. B., Shafi, S., Murugaesu, N., Mitter, R., Akarca, A. U., Linares, J., Marafioti, T., Henry, J. Y., Van Allen, E. M., Miao, D., . . . Swanton, C. (2016). Clonal neoantigens elicit T cell immunoreactivity and sensitivity to immune checkpoint blockade. Science (New York, N.Y.), 351(6280), 1463-1469.
Landau D A, Carter S L, Stojanov P, McKenna A, Stevenson K, Lawrence M S, Sougnez C, Stewart C, Sivachenko A, Wang L, Wan Y, Zhang W, Shukla S A, Vartanov A, Fernandes S M, Saksena G, Cibulskis K, Tesar B, Gabriel S, Hacohen N, Meyerson M, Lander E S, Neuberg D, Brown J R, Getz G, Wu C J. Evolution and impact of subclonal mutations in chronic lymphocytic leukemia. Cell. 2013 Feb. 14; 152(4):714-26. doi: 10.1016/j.cell.2013.01.019.
Raine K M, Van Loo P, Wedge D C, Jones D, Menzies A, Butler A P, Teague J W, Tarpey P, Nik-Zainal S, Campbell P J. ascatNgs: Identifying Somatically Acquired Copy-Number Alterations from Whole-Genome Sequencing Data. Curr Protoc Bioinformatics. 2016 Dec. 8; 56:15.9.1-15.9.17. doi: 10.1002/cpbi.17.
Heemskerk B, Kvistborg P, Schumacher TNM. The cancer antigenome. The EMBO Journal Vol. 32, No. 2, 2013.
Castel, S E, Levy-Moonshine A, Mohammadi P, Banks E, Lappalainen T. Tools and best practice for data processing in allelic expression analysis. Genome Biology (2015) 16:195.
Favero F, Joshi T, Marquard A M, Birkbak N J, Krzystanek M, Li Q, Szallasi Z, Eklund A C. Sequenza: allele-specific copy number and mutation profiles from tumor sequencing data. Ann Oncol. 2015 January; 26(1):64-70.

All references cited herein are incorporated herein by reference in their entirety and for all purposes to the same extent as if each individual publication or patent or patent application was specifically and individually indicated to be incorporated by reference in its entirety.

The specific embodiments described herein are offered by way of example, not by way of limitation. Various modifications and variations of the described compositions, methods, and uses of the technology will be apparent to those skilled in the art without departing from the scope and spirit of the technology as described. Any sub-titles herein are included for convenience only and are not to be construed as limiting the disclosure in any way. Unless context dictates otherwise, the descriptions and definitions of the features set out above are not limited to any particular aspect or embodiment of the invention and apply equally to all aspects and embodiments which are described.

The methods of any embodiments described herein may be provided as computer programs or as computer program products or computer readable media carrying a computer program which is arranged, when run on a computer, to perform the method(s) described above.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

It must be noted that, as used in the specification and the appended claims, the singular forms “a,” “an,” and “the” include plural referents unless the context clearly dictates otherwise.

Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example+/−10%.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

Other aspects and embodiments of the invention provide the aspects and embodiments described above with the term “comprising” replaced by the term “consisting of” or “consisting essentially of”, unless the context dictates otherwise.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

1. A method of determining whether a tumour-specific mutation is likely to be expressed in a subject, the method comprising:
obtaining RNA sequence data from one or more samples from the subject comprising tumour genetic material, the RNA sequence data comprising for each of the one or more samples, the number of RNA reads in the sample that show the tumour-specific mutation (b), and the total number of RNA reads at the location of the tumour-specific mutation (d);
determining the likelihoods of the sequence data if the tumour-specific mutation is (i) expressed and (ii) not-expressed; and
comparing the likelihoods obtained thereby determining whether the tumour-specific mutation is likely to be expressed in the subject.

2. The method of claim 1, wherein comparing the likelihoods comprises determining:
(a) the posterior probability that the tumour-specific mutation is expressed depending on:
a prior probability of the mutation being expressed (μ_{ρ}), and
the likelihoods of the sequence data if the tumour-specific mutation is (i) expressed and (ii) not-expressed; and/or
(b) determining the power to detect whether the tumour-specific mutation is expressed at a predetermined false positive rate, wherein the power to detect whether the tumour-specific mutation is expressed is the area under the curve of the likelihood of the number of reads that show the tumour specific mutation if the tumour-specific mutation is expressed as a function of the number of RNA reads in the sample that show the tumour-specific mutation (b), above a threshold number of reads (b_{c}),
wherein the threshold number of reads (b_{c}) is the number of reads such that: the area under the curve of the likelihood of the number of reads that show the tumour specific mutation if the tumour-specific mutation is not expressed as a function of the number of RNA reads in the sample that show the tumour-specific mutation (b) above the threshold number of reads is equal to the predetermined false positive rate.

3. The method of any preceding claim, wherein the likelihoods of the sequence data are the probabilities of the sequence data in view of a tumour fraction for each of the one or more samples (t), and the fraction (α) of the total number of reads at the location of the tumour-specific mutation that originate from a population of cells in the one or more samples that does not comprise the tumour-specific mutation.

4. The method of any preceding claim, wherein each sample is assumed to comprise: a first population of cells that each have a genotype (G_{V}) comprising at least one copy of the tumour-specific mutation, and a second population of cells that each have a genotype that does not comprise the tumour-specific mutation (G_{N}), the first population of cells representing a proportion equal to the tumour fraction (t) for the respective sample.

5. The method of any preceding claim, wherein the tumour-specific mutation is assumed to be ubiquitous in the one or more samples, and/or wherein the tumour-specific mutation is a clonal mutation or a mutation assumed to be clonal in the tumour of the subject.

6. The method of any preceding claim, wherein the RNA sequence data comprises RNA sequence data from a plurality of samples from the subject, optionally from a plurality of tumour samples from the subject, and wherein the posterior probability that the tumour specific mutation is expressed is a posterior probability that the tumour specific mutation is expressed ubiquitously in a tumour of the subject.

7. The method of any preceding claim, wherein the likelihoods of the sequence data if the tumour-specific mutation is (i) expressed and (ii) not-expressed depend on the fraction (a) of the total expression of the gene comprising the tumour-specific mutation that originate from a population of cells in the one or more samples that does not comprise the tumour-specific mutation, wherein α is set to a predetermined default value or wherein α is estimated from data comprising RNA sequence data for a plurality of samples having different tumour fractions.

8. The method of claim 7, wherein if the genotype of a population of cells in the one or more samples that have a genotype comprising at least one copy of the tumour-specific mutation does not have any non-mutant alleles at the locus of the tumour-specific mutation, α is set to 1 when determining the likelihood of the sequence data if the tumour-specific mutation is not-expressed.

9. The method of claim 7 or claim 8, wherein α is estimated from data comprising RNA sequence data for a plurality of samples having different tumour fractions, wherein α is derived from the slope (g) of a linear regression model fitted to the total expression of the gene comprising the tumour-specific mutation in a plurality of samples as a function of their tumour purities, optionally wherein α is provided by:
$$α = \frac{1}{2} ( 1 - \frac{g}{T P {M}^{T} + T P {M}^{N}} ) \qquad ( 31 )$$
where TPM^{T} and TPM^{N} are the total expression values for the gene for a tumour sample and a normal sample, respectively, optionally wherein TPM^{T} and TPM^{N} are obtained using a regression model fitted to values of the total expression for the gene (TPM) in a plurality of tumour samples with at least two different tumour fractions (t), as a function of tumour fraction.

10. The method of any of claims 2 to 9, wherein the prior probability of the tumour-specific mutation being expressed may be the mean (μ_{ρ}) of a Beta distribution (p(ρ) for parameter ρ of a Bernoulli distribution for the variable capturing whether the tumour-specific mutation is expressed (E), wherein μ_{ρ} is set to a predetermined value.

11. The method of claim 10, wherein μ_{ρ} is set to a first predetermined value when the total number of RNA reads at the location of the tumour-specific mutation is at or above, or above a predetermined threshold, optionally wherein the predetermine threshold is 0 (d>0), and/or wherein when the total number of RNA reads at the location of the tumour-specific mutation is below a predetermined threshold, μ_{ρ} is set to a first predetermined value or a second predetermined value depending on whether the gene comprising the tumour-specific mutation is expected to be expressed, optionally wherein the gene comprising the tumour-specific mutation is considered as expected to be expressed if the total expression of the gene in the sample is above a predetermined threshold, and considered as expected not to be expressed otherwise, and/or optionally wherein the first predetermined value is 0.5 and/or the second predetermined value is below 0.5.

12. The method of any preceding claim, wherein the likelihoods are conditional on the tumour purity of the one or more samples, optionally wherein the tumour purity of the one or more samples is estimated using genomic sequence data from the one or more samples.

13. The method of any of claims 4 to 12, wherein the probability of observing the sequence data depends on the genotypes of the first and second populations of cells, the total number of reads at the locus of the tumour specific mutation, the tumour fraction (t), the fraction (α) of the total expression for the gene comprising the tumour-specific mutation that originate from the second population of cells, and the ratio of expression of the alleles not comprising the tumour-specific mutation relative to the total expression at the locus of the tumour-specific mutation (θ).

14. The method of claim 13, wherein the ratio of expression of the alleles not comprising the tumour-specific mutation relative to the total expression at the locus of the tumour-specific mutation (θ) is assumed to be a random variable with a first distribution if the tumour-specific mutation is expressed and a second distribution if the tumour-specific mutation is not expressed, and/or wherein the likelihoods are marginal likelihoods obtained from the probability of observing the sequence data by integrating out the ratio of expression of the alleles not comprising the tumour-specific mutation relative to the total expression at the locus of the tumour-specific mutation (θ).

15. The method of claim 14, wherein the second distribution is a beta distribution with parameters α₀, β₀, and the first distribution is a beta distribution with parameters α₁, β₁, optionally wherein α₀>1 (e.g. α₀=9999), β₀=1, α₁=1, and β₁=1.

16. The method of any of claims 13 to 15, wherein the probability of observing the sequence data conditional on the genotypes of the first and second populations of cells (G_{V},G_{N}), the total number of reads at the locus of the tumour specific mutation, the tumour fraction (t), the fraction (α) of the total expression of the gene comprising the tumour-specific mutation that originate from the second population of cells, and the ratio of expression of the alleles not comprising the tumour-specific mutation relative to the total expression at the locus of the tumour-specific mutation (θ) is assumed to follow a binomial distribution with a parameter ξ(G,θ,α,t) representing the probability of sampling a read with the tumour specific mutation from a tumour sample.

17. The method of claim 16, wherein ξ(G,θ,α,t) is given by any of equations (2), (2′), (2″), optionally wherein
$$ξ ( G , θ , α , t ) = \frac{α ( 1 - t ) c ( {G}_{N} )}{α ( 1 - t ) c ( {G}_{N} ) + ( 1 - α ) t c ( {G}_{V} )} μ ( {G}_{N} , θ , ε ) + \frac{( 1 - α ) t c ( {G}_{V} )}{α ( 1 - t ) c ( {G}_{N} ) + ( 1 - α ) t c ( {G}_{V} )} μ ( {G}_{V} , θ , ε ) \qquad ( 2 ’ ’ )$$
where c(G_{V}) and c(G_{N}) are respectively the total number of copies of the locus comprising the tumour-specific mutation in the first and second populations of cells,ε is a sequencing error rate, μ(G_{N},θ,ε) is the probability of sampling a read with the tumour-specific mutation from the second population of cells and μ(G_{V},θ,ε) is the probability of sampling a read with the tumour-specific mutation from the first population of cells.

18. The method of claim 17, wherein, μ(G_{N},θ,ε) and μ(G_{V},θ,ε) are provided by equation (1), respectively with G=G_{N} and G=G_{V} being the genotype of the second and first population of cells, and b(G) being the number of copies of the allele comprising the tumour-specific mutation in the cells of the respective population of cells.

19. The method of any of claims 2 to 18, wherein the posterior probability depends on the ratio (r) of the likelihoods of the sequence data if the tumour-specific mutation is (i) expressed (P(b,d|α,t,E=1)) and (ii) not-expressed (P(b,d|α,t,E=0)), and/or wherein the posterior probability is given by equation (13), (14) or (14′), where μ_{ρ} is the prior probability of the mutation being expressed.

20. The method of any preceding claim, further comprising:
repeating the method for a plurality of tumour-specific mutations identified in the subject and optionally ranking or otherwise prioritising the plurality of tumour-specific mutations at least in part based on their determined probability of being expressed in the subject, and/or identifying one or more tumour-specific mutations in the subject, and/or
sequencing one or more samples from the subject comprising tumour genetic material to obtain RNA sequence reads and optionally DNA sequence reads.

21. A method of identifying one or more neoantigens in a subject, the method comprising:
identifying a plurality of tumour-specific mutations in the subject;
determining whether one or more of the tumour-specific mutations is likely to be expressed in a tumour of the subject using the method of any of claims 1 to 21; and
determining whether one or more of the tumour-specific mutations is likely to give rise to a neoantigen, wherein a neoantigen is a tumour-specific mutation that satisfies one or more predetermined criteria on whether the tumour-specific mutation is likely to be expressed in the tumour.

22. A method of providing an immunotherapy for a subject that has been diagnosed as having cancer, the method comprising:
identifying one or more tumour-specific mutations in the subject;
determining whether one or more of the tumour-specific mutations is likely to be expressed in a tumour of the subject using the method of any of claims 1 to 21;
selecting one or more tumour-specific mutations from the identified tumour-specific mutations based on the result of the determining and optionally one or more further criteria; and
designing an immunotherapy that targets one or more neoantigens derived from the selected one or more tumour-specific mutations.

23. A method of treating a subject that has been diagnosed as having cancer, the method comprising: identifying one or more neoantigens by:
identifying a plurality of tumour-specific mutations in the subject;
determining whether one or more of the tumour-specific mutations is likely to be expressed in the subject;
selecting one or more of the tumour-specific mutations as candidate neoantigens, wherein a candidate neoantigen is a tumour-specific mutation that satisfies at least one or more predetermined criteria on whether the tumour-specific mutation is likely to be expressed; and
treating the subject with an immunotherapy that targets one or more of the selected candidate neoantigens;
wherein determining whether a tumour-specific mutation is likely to be expressed in a subject, comprises: obtaining, by a processor, RNA sequence data from one or more samples from the subject comprising tumour genetic material, the RNA sequence data comprising for each of the one or more samples: the number of reads in the sample that show the tumour-specific mutation (b) and the total number of reads at the location of the tumour-specific mutation (d), and determining, by the processor, the probabilities of observing the RNA sequence data if the tumour-specific mutation is (i) expressed and (ii) not expressed.

24. A system comprising: a processor; and a computer readable medium comprising instructions that, when executed by the processor, cause the processor to perform the steps of any method described herein, such as a method according to any of claims 1 to 22.

25. One or more non-transitory computer readable media comprising instructions that, when executed by one or more processors, cause the one or more processors to perform the steps of any method described herein, such as a method according to any of claims 1 to 22.
